(19) United States
(12) Patent Application Publication
(10) Pub. No.: US 2019/0380607 A1
(43) Pub. Date: Dec. 19, 2019

(54) Mobile Wearable Device for Measuring Electromagnetic Brain Activity

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 16/554,029

(22) Filed: Aug. 28, 2019

Related U.S. Application Data
(63) Continuation-in-part of application No. 16/022,987, filed on Jun. 29, 2018.
Application No. 16/022,987 is a continuation-in-part of application No. 15/136,948, filed on Apr. 24, 2016, now Pat. No. 10,234,942.
Application No. 15/136,948 is a continuation-in-part of application No. 14/599,522, filed on Jan. 18, 2015, now Pat. No. 9,814,426.
Application No. 14/599,522 is a continuation-in-part of application No. 14/562,719, filed on Dec. 7, 2014, now Pat. No. 10,130,277.
(60) Provisional application No. 62/851,904, filed on May 23, 2019.
(60) Provisional application No. 62/796,901, filed on Jan. 25, 2019.
(60) Provisional application No. 62/791,838, filed on Jan. 13, 2019.
(60) Provisional application No. 62/089,696, filed on Dec. 9, 2014.
(60) Provisional application No. 61/939,244, filed on Feb. 12, 2014.
(60) Provisional application No. 61/932,517, filed on Jan. 28, 2014.

(51) Int. Cl. A61B 5/04; A61B 5/00

(52) U.S. Cl. CPC A61B 5/04008; A61B 5/6803

(57) ABSTRACT

This invention is a mobile wearable device for measuring electromagnetic brain activity. It can be embodied in a headband or eyewear with soft prongs and/or teeth which protrude into and/or under the person's hair. The prongs and/or teeth can be made from a silicone material (such as polydimethylsiloxane) which has been impregnated, doped, coated, or embedded with conductive material (such as a metal and/or carbon) in order to provide consistent but comfortable contact with the person's head.

### CROSS-REFERENCE TO RELATED APPLICATIONS

This application claims the priority benefit of provisional application 62/851,904 by Robert A. Connor filed on 2019 May 23. This application also claims the priority benefit of provisional application 62/796,901 by Robert A. Connor filed on 2019 Jan. 25. This application also claims the priority benefit of provisional application 62/791,838 by Robert A. Connor filed on 2019 Jan. 13. This application also is a continuation in part of application Ser. No. 16/022,987 by Robert A. Connor filed on 2018 Jun. 29. Application Ser. No. 16/022,987 was a continuation in part of application Ser. No. 15/136,948 by Robert A. Connor filed on 2016 Apr. 24. Application Ser. No. 15/136,948 was a continuation in part of application Ser. No. 14/599,522 by Robert A. Connor filed on 2015 Jan. 18 which issued as U.S. Pat. No. 9,814,426 on 2017 Nov. 14. Application Ser. No. 14/599,522 claimed the priority benefit of provisional application 62/089,696 by Robert A. Connor filed on 2014 Dec. 9. Application Ser. No. 14/599,522 also was a continuation in part of application Ser. No. 14/562,719 by Robert A. Connor filed on 2014 Dec. 7 which issued as U.S. Pat. No. 10,130,277 on 2018 Nov. 20. Application Ser. No. 14/599,522 also claimed the priority benefit of provisional application 61/939,244 by Robert A. Connor filed on 2014 Feb. 12. Application Ser. No. 14/599,522 also claimed the priority benefit of provisional application 61/932,517 by Robert A. Connor filed on 2014 Jan. 28. The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable devices for measuring electromagnetic brain activity.

### Introduction

Disclosed herein is a mobile wearable device for measuring electromagnetic brain activity. There are numerous potential applications for a mobile wearable device for measuring electromagnetic brain activity in the fields of: medicine, health, and fitness; communication and telepresence; entertainment, augmented reality (AR), virtual reality (VR), and gaming; sports; navigation; telerobotics; and defense. For example, such a mobile wearable device can serve as a method of communication by people who are paralyzed or have otherwise lost muscle control. As another example, such a device can be used as a brain to computer interface (BCI) in situations where other forms of human-to-computer interaction (such as a touch-based interface or a speech-based interface) are undesirable, difficult, or even impossible. As another example, such a device can help to predict when a person may have a seizure (such as an epileptic seizure), which can enable the person to prepare for (or potentially even avoid) the seizure. As another example, a comfortable mobile wearable device can be used to monitor EEG patterns while a person sleeps in order to diagnose sleep conditions, provide selected feedback, and/or improve sleep quality.

A mobile wearable device for measuring brain activity can be specified based on the geometry of the device, including the configurations/shapes of different arms/portions of the device which span the person's head. Additionally, a mobile wearable device for measuring brain activity can be specified based on the locations of electromagnetic energy sensors which are held on the person's head. In an example, electromagnetic energy sensors can be EEG sensors. Sensor locations can be specified using the electrode locations of the internationally-recognized Modified Combinatorial Nomenclature (MCN) electrode placement system.

### Review of the Relevant Art

It can be challenging trying to classify relevant art in this field into discrete categories. However, classification of relevant art into categories, even if imperfect, can be an invaluable tool for reviewing the relevant art. Towards this end, I herein identify 14 categories of relevant art and provide examples of relevant art in each category (including patent or patent application number, inventor, publication date, and title). Some examples of relevant art disclose multiple concepts and thus appear in more than one category.

The 14 categories of relevant art which are used for this review are as follows: (1) device like a skull cap with EEG/brainwave sensors; (2) device like a baseball cap with EEG/brainwave sensors; (3) device with [multiple] front-to-back arcuate members and EEG/brainwave sensors; (4) device with [multiple] side-to-side arcuate members and EEG/brainwave sensors; (5) device with multiple cross-crossing arcuate members and EEG/brainwave sensors; (6) device with multiple arms radially-extending from side and EEG/brainwave sensors; (7) device with multiple arms radially-downward from top and EEG/brainwave sensors; (8) device with multiple arms radially-forward from rear and EEG/brainwave sensors; (9) device with multiple arms radially-backward from front and EEG/brainwave sensors; (10) device with circular horizontal loop (e.g. headband style) and EEG/brainwave sensors; (11) device with top semicircular loop (e.g. headphone style) and EEG/brainwave sensors; (12) device with rear semicircular loop and EEG/brainwave sensors; (13) device with frontal semicircular loop and EEG/brainwave sensors; and (14) device like eyeglasses or other eyewear with EEG/brainwave sensors.

1. Device Like a Skull Cap with EEG/Brainwave Sensor(s)

Devices in this category hold electromagnetic brain activity sensors in contact with (or proximity to) a person's head using a sensor-positioning member configured like a skull cap. A “skull cap” is defined herein as a wearable cap-like or hat-like member that covers most of the hair-covered portion of a person's head and flexibly conforms to the contours of the head. A skull cap is generally held snugly against the surface of the head by straps and/or elastic bands. Due to the high percentage of the surface area of the top of a person's head which is covered by such a device, such a device can be used to hold a relatively large number of electromagnetic brain activity sensors in a variety of positions across a person's head. Most of the devices in this category are relatively obtrusive and would be awkward to wear outside a medical setting. They are generally not appropriate for wearing during the activities of daily life.

Prior art which appears to be within this category includes U.S. Pat. No. 4,537,198 (Corbett, Aug. 27, 1985, “Electrode Cap”), U.S. Pat. No. 4,632,122 (Johansson, Dec. 30, 1986, “Method and Apparatus for Conducting Brain Function Diagnostic Test”), U.S. Pat. No. 4,683,892 (Johansson, Aug. 4, 1987, “Method and Apparatus for Conducting Brain Function Diagnostic Test”), U.S. Pat. No. 4,709,702 (Sherwin, Dec. 1, 1987, “Electroencephalographic Cap”), U.S. Pat. No. 4,800,888 (Itil et al., Jan. 31, 1989, “Enhanced Electrode Headset”), U.S. Pat. No. 5,038,782 (Gevins et al., Aug. 13, 1991, “Electrode System for Brain Wave Detection”), U.S. Pat. No. 5,724,987 (Gevins et al., Mar. 10, 1998, “Neurocognitive Adaptive Computer-Aided Training Method and System”), U.S. Pat. No. 6,067,464 (Musha, 5/23/200, “Electrode”), U.S. Pat. No. 8,155,736 (Sullivan et al., Apr. 10, 2012, “EEG Control of Devices Using Sensory Evoked Potentials”), and U.S. Pat. No. 8,391,966 (Luo et al., Mar. 5, 2013, “Sensory-Evoked Potential (SEP) Classification/Detection in the Time Domain”).

Prior art which appears to be within this category also includes U.S. patent applications: 20070225577 (Mathan, Sep. 27, 2007, “System and Method for Providing Sensor Based Human Factors Protocol Analysis”), 20070225585 (Washbon and Delic, Sep. 27, 2007, “Headset for Electrodes”), 20070238945 (Delic et al., Oct. 11, 2007, “Electrode Headset”), 20070255127 (Mintz et al., Nov. 1, 2007, “Mobile Electroencephalograph Data Collection and Diagnosis System”), 20080275359 (Mintz et al., Nov. 6, 2008, “Mobile in Vivo Brain Scan and Analysis System”), 20100234752 (Sullivan et al., Sep. 16, 2010, “EEG Control of Devices Using Sensory Evoked Potentials”), 20110040202 (Luo et al., Feb. 17, 2011, “Sensory-Evoked Potential (SEP) Classification/Detection in the Time Domain”), 20110298706 (Mann, Dec. 8, 2011, “Brainwave Actuated Apparatus”), and 20120059273 (Meggiolaro et al., Mar. 8, 2012, “Process and Device for Brain Computer Interface”).

Prior art which appears to be within this category also includes U.S. patent applications: 20120136273 (Michelson Jr., May 31, 2012, “Apparatus and Method for Monitoring and Analyzing Brainwaves”), 20120220889 (Sullivan et al., Aug. 30, 2012, “EEG Control of Devices Using Sensory Evoked Potentials”), 20120289869 (Tyler, Nov. 15, 2012, “Devices and Methods for Modulating Brain Activity”), 20130211276 (Luo et al., Aug. 15, 2013, “Sensory-Evoked Potential (SEP) Classification/Detection in the Time Domain”), 20130281759 (Hagedorn et al., Oct. 24, 2013, “Transcranial Stimulation Device and Method Based on Electrophysiological Testing”), 20140163408 (Kocher, Jun. 12, 2014, “System for Analyzing Mental and Behavioral Correlations”), and 20140288614 (Hagedorn et al., Sep. 25, 2014, “Electrophysiology Measurement and Training and Remote Databased and Data Analysis Measurement Method and System”).

2. Device Like a Baseball Cap with EEG/Brainwave Sensor(s)

Devices in this category hold electromagnetic brain activity sensors in contact with (or proximity to) a person's head using a sensor-positioning member configured like a baseball cap. A “baseball cap” is defined herein as a wearable member with a generally-hemispherical portion which fits over the top of a person's head and an attached visor which extends outward from the person's forehead. Sometimes arcuate bands are embedded in the cap in circular-around-the-head, side-to-side-over-the-top, and/or front-to-back-over-the-top configurations. Devices in this category generally do not cover as much of the surface of the head and may not fit as tightly against the surface of the head as skull cap devices. On the plus side, devices in this category are generally less obtrusive than skull cap devices. However, there are still many circumstances and settings wherein wearing a baseball cap is inappropriate.

Prior art which appears to be within this category includes U.S. Pat. No. 4,697,598 (Bernard et al., Oct. 6, 1987, “Evoked Potential Autorefractometry System”), U.S. Pat. No. 4,709,702 (Sherwin, Dec. 1, 1987, “Electroencephalographic Cap”), U.S. Pat. No. 6,161,030 (Levendowski et al., Dec. 12, 2000, “Portable EEG Electrode Locator Headgear”), U.S. Pat. No. 6,381,481 (Levendowski et al., Apr. 30, 2002, “Portable EEG Electrode Locator Headgear”), U.S. Pat. No. 6,574,513 (Collura et al., Jun. 3, 2003, “EEG Electrode Assemblies”), U.S. Pat. No. 6,640,122 (Manoli et al., Oct. 28, 2003, “EEG Electrode and EEG Electrode Locator Assembly”), and U.S. Pat. No. 7,204,250 (Burton, Apr. 17, 2007, “Bio-Mask”), U.S. Pat. No. 8,281,787 (Burton, Oct. 9, 2012, “Bio-Mask with Integral Sensors”).

Prior art which appears to be within this category also includes U.S. patent applications: 20020029005 (Levendowski et al., Mar. 7, 2002, “Portable EEG Electrode Locator Headgear”), 20040073129 (Caldwell et al., Apr. 15, 2004, “EEG System for Time-Scaling Presentations”), 20040163648 (Burton, Aug. 26, 2004, “Bio-Mask with Integral Sensors”), 20100147304 (Burton, Jun. 17, 2010, “Bio-Mask with Integral Sensors”), 20110015503 (Joffe et al., Jan. 20, 2011, “Medical Apparatus for Collecting Patient Electroencephalogram (EEG) Data”), 20110046502 (Pradeep et al., Feb. 24, 2011, “Distributed Neuro-Response Data Collection and Analysis”), 20110046504 (Pradeep et al., Feb. 24, 2011, “Distributed Neuro-Response Data Collection and Analysis”), and 20110270117 (Warwick et al., Nov. 3, 2011, “Remote Continuous Seizure Monitor and Alarm”).

3. Device with [Multiple] Front-to-Back Arcuate Member(s) and EEG/Brainwave Sensor(s)

Devices in this category hold electromagnetic brain activity sensors in contact with (or proximity to) a person's head using (multiple) arcing member(s) which span a person's head from front-to-back (or vice versa). Devices in this category can look similar to some types of bicycle helmets with front-to-back arcuate members. In an example, the front-to-back arcing members can converge at the forehead and at the rear of the head. In an example, a device in this category can comprise: a first arcuate member which encircles a person's head: a second arcuate member which loops front-to-back over the top of the head; and third and fourth arcuate members which loop front-to-back over the sides of the head between the first and second members. Devices in this category can hold a relatively large number of electromagnetic brain activity sensors along arcuate front-to-rear lines on a person's head. However, such devices tend to be too obtrusive to wear during the activities of daily life.

Prior art which appears to be within this category includes U.S. Pat. No. 3,998,213 (Price, Dec. 21, 1976, “Self-Adjustable Holder for Automatically Positioning Electroencephalographic Electrodes”), U.S. Pat. No. 8,355,769 (Levendowski et al., Jan. 15, 2013, “System for the Assessment of Sleep Quality in Adults and Children”), U.S. Pat. No. 8,463,354 (Fadem, Jun. 11, 2013, “Electrode System with Rigid-Flex Circuit”), U.S. Pat. No. 8,639,313 (Westbrook et al, Jan. 28, 2014, “System for the Assessment of Sleep Quality in Adults and Children”); and U.S. patent applications 20100125190 (Fadem, May 20, 2010, “Electrode System”), 20100240982 (Westbrook et al., Sep. 23, 2010, “System for the Assessment of Sleep Quality in Adults and Children”), and 20130131464 (Westbrook et al., May 23, 2013, “System for the Assessment of Sleep Quality in Adults and Children”).

4. Device with [Multiple] Side-to-Side Arcuate Member(s) and EEG/Brainwave Sensor(s)

Devices in this category hold electromagnetic brain activity sensors in contact with (or proximity to) a person's head using (multiple) arcing member(s) which span a person's head from side to side. In an example, side-to-side arcing members can converge near, or over, the person's ears. In an example, devices in this category can be similar to those in the previous category, except having been rotated 90 degrees so that the arcuate members converge on the sides of the person's head rather than the front and rear of the person's head. Devices in this category can hold a relatively large number of electromagnetic brain activity sensors along arcuate side-to-side lines on a person's head. However, such devices tend to be too obtrusive to wear during the activities of daily life.

Prior art which appears to be within this category includes U.S. Pat. No. 4,836,219 (Hobson et al., Jun. 6, 1989, “Electronic Sleep Monitor Headgear”), U.S. Pat. No. 5,800,351 (Mann, Sep. 1, 1998, “Electrode Supporting Head Set”), U.S. Pat. No. 6,574,513 (Collura et al., Jun. 3, 2003, “EEG Electrode Assemblies”), U.S. Pat. No. 7,158,822 (Payne Jr., Jan. 2, 2007, “Electrode Holder, Headwear, and Wire Jacket Adapted for Use in Sleep Apnea Testing”), and U.S. Pat. No. 7,885,706 (Ludvig et al., Feb. 8, 2011, “System and Device for Seizure Detection”).

Prior art which appears to be within this category also includes U.S. patent applications: 20030018278 (Jordan, Jan. 23, 2003, “Electroencephalogram Acquisition Unit and System”), 20050277821 (Payne, Dec. 15, 2005, “Electrode Holder, Headwear, and Wire Jacket Adapted for Use in Sleep Apnea Testing”), 20070112262 (Payne, May 17, 2007, “Electrode Holder, Headwear, and Wire Jacket Adapted for Use in Sleep Apnea Testing”), 20080082019 (Ludving et al., Apr. 3, 2008, “System and Device for Seizure Detection”), 20090281446 (Ludvig et al., Nov. 12, 2009, “System and Device for Seizure Detection”), 20110015503 (Joffe et al., Jan. 20, 2011, “Medical Apparatus for Collecting Patient Electroencephalogram (EEG) Data”), and 20110270117 (Warwick et al., Nov. 3, 2011, “Remote Continuous Seizure Monitor and Alarm”).

5. Device with Multiple Cross-Crossing Arcuate Members and EEG/Brainwave Sensor(s)

Devices in this category hold electromagnetic brain activity sensors in contact with (or proximity to) a person's head using multiple arcing members which span a person's head from front-to-rear and also multiple arcing members which span a person's head from side-to-side. In an example, the front-to-rear arcuate members and the side-to-side arcuate members can form a criss-cross pattern on the person's head. Devices in this category can hold a relatively large number of electromagnetic brain activity sensors on a person's head. However, such devices tend to be too obtrusive to wear during the activities of daily life.

Prior art which appears to be within this category includes U.S. Pat. No. 3,998,213 (Price, Dec. 21, 1976, “Self-Adjustable Holder for Automatically Positioning Electroencephalographic Electrodes”), U.S. Pat. No. 5,293,867 (Oommen, Mar. 15, 1994, “Method and Apparatus for Marking Electrode Locations for Electroencephalographic Procedure”), U.S. Pat. No. 5,479,934 (Imran, Jan. 2, 1996, “EEG Headpiece with Disposable Electrodes and Apparatus and System and Method for Use Therewith”), U.S. Pat. No. 6,488,617 (Katz, Dec. 3, 2002, “Method and Device for Producing a Desired Brain State”), U.S. Pat. No. 8,463,354 (Fadem, Jun. 11, 2013, “Electrode System with Rigid-Flex Circuit”); and U.S. patent applications 20030018278 (Jordan, Jan. 23, 2003, “Electroencephalogram Acquisition Unit and System”), and 20100125190 (Fadem, May 20, 2010, “Electrode System”).

6. Device with Multiple Arms Radially-Extending from Side and EEG/Brainwave Sensor(s)

Devices in this category hold electromagnetic brain activity sensors in contact with (or proximity to) a person's head using multiple sensor-holding protrusions, fingers, or arms which extend radially outward from a central position on one side (or from central positions on both sides) of a person's head. In an example, such devices can include bilateral clusters (one on each side of the head) of radially-extending protrusions, fingers, or arms. In an example, radially-extending protrusions, fingers, or arms can curve around the head toward the front, top, and/or rear portions of the head. To use colorful language, some such devices can look like a wearer has one or two starfish (or even octopi) clinging to the sides of their head. Such devices can be less obtrusive than those in the preceding categories (especially when they do not span the forehead or the top of the head), but can still attract attention if worn during the activities of daily life.

Prior art which appears to be within this category includes U.S. Pat. No. 5,954,667 (Finkenzeller et al., Sep. 21, 1999, “Device for Deriving Acoustically Evoked Brain Potentials”), U.S. Pat. No. 8,271,075 (Chuang et al., Sep. 18, 2012, “Audio Headset with Bio-Signal Sensors”), U.S. Pat. No. 8,392,250 (Pradeep et al., Mar. 5, 2013, “Neuro-Response Evaluated Stimulus in Virtual Reality Environments”), U.S. Pat. No. 8,392,251 (Pradeep et al., Mar. 5, 2013, “Location Aware Presentation of Stimulus Material”), U.S. Pat. No. 8,396,744 (Pradeep et al., Mar. 12, 2013, “Effective Virtual Reality Environments for Presentation of Marketing Materials”), U.S. Pat. No. 8,548,852 (Pradeep et al., Oct. 1, 2013, “Effective Virtual Reality Environments for Presentation of Marketing Materials”), and U.S. Pat. No. 8,655,428 (Pradeep et al., Feb. 18, 2014, “Neuro-Response Data Synchronization”).

Prior art which appears to be within this category also includes U.S. patent applications: 20070106169 (Fadem, May 10, 2007, “Method and System for an Automated E.E.G. System for Auditory Evoked Responses”), 20070191727 (Fadem, Aug. 16, 2007, “Evoked Response Testing System for Neurological Disorders”), 20070225585 (Washbon and Delic, Sep. 27, 2007, “Headset for Electrodes”), 20070238945 (Delic et al., Oct. 11, 2007, “Electrode Headset”), 20080208072 (Fadem et al., Aug. 28, 2008, “Biopotential Waveform Data Fusion Analysis and Classification Method”), 20110237971 (Pradeep et al., Sep. 29, 2011, “Discrete Choice Modeling Using Neuro-Response Data”), and 20110282231 (Pradeep et al., Nov. 17, 2011, “Mechanisms for Collecting Electroencephalography Data”).

Prior art which appears to be within this category also includes U.S. patent applications: 20110282232 (Pradeep et al., Nov. 17, 2011, “Neuro-Response Data Synchronization”), 20120072289 (Pradeep et al., Mar. 22, 2012, “Biometric Aware Content Presentation”), 20130131537 (Tam, May 23, 2013, “Tong Ren Brainwave Entrainment”), 20130185144 (Pradeep et al., Jul. 18, 2013, “Systems and Methods for Analyzing Neuro-Response Data and Virtual Reality Environments”), 20130314243 (Le, Nov. 28, 2013, “System and Method for Enabling Collaborative Analysis of a Biosignal”), 20130317382 (Le, Nov. 28, 2013, “System and Method for Providing and Aggregating Biosignals and Action Data”), and 20130317384 (Le, Nov. 28, 2013, “System and Method for Instructing a Behavior Change in a User”).

7. Device with Multiple Arms Radially-Downward from Top and EEG/Brainwave Sensor(s)

Devices in this category hold electromagnetic brain activity sensors in contact with (or proximity to) a person's head using multiple sensor-holding protrusions, fingers, or arms which extend radially downward from a position on the top of a person's head. In an example, radially-extending protrusions, fingers, or arms can curve around the head toward the front, sides, and/or rear portions of the head. To use the colorful language from the previous category, now a figurative starfish (or octopus) is clinging to the top of the person's head. Such devices can be less obtrusive than some of those in the preceding categories, but can still attract attention if worn during the activities of daily life.

Prior art which appears to be within this category includes U.S. Pat. No. 6,067,464 (Musha, 5/23/200, “Electrode”), U.S. Pat. No. 6,154,669 (Hunter et al., Nov. 28, 2000, “Headset for EEG Measurements”), U.S. Pat. No. 6,161,030 (Levendowski et al., Dec. 12, 2000, “Portable EEG Electrode Locator Headgear”), U.S. Pat. No. 6,381,481 (Levendowski et al., Apr. 30, 2002, “Portable EEG Electrode Locator Headgear”), U.S. Pat. No. 7,551,952 (Gevins et al., Jun. 23, 2009, “EEG Electrode Headset”), U.S. Pat. No. 8,103,328 (Turner et al., Jan. 24, 2012, “Self-Locating Sensor Mounting Apparatus”), U.S. Pat. No. 8,392,250 (Pradeep et al., Mar. 5, 2013, “Neuro-Response Evaluated Stimulus in Virtual Reality Environments”), U.S. Pat. No. 8,392,251 (Pradeep et al., Mar. 5, 2013, “Location Aware Presentation of Stimulus Material”), U.S. Pat. No. 8,396,744 (Pradeep et al., Mar. 12, 2013, “Effective Virtual Reality Environments for Presentation of Marketing Materials”), U.S. Pat. No. 8,548,852 (Pradeep et al., Oct. 1, 2013, “Effective Virtual Reality Environments for Presentation of Marketing Materials”), and U.S. Pat. No. 8,655,428 (Pradeep et al., Feb. 18, 2014, “Neuro-Response Data Synchronization”).

Prior art which appears to be within this category also includes U.S. patent applications: 20020029005 (Levendowski et al., Mar. 7, 2002, “Portable EEG Electrode Locator Headgear”), 20070093706 (Gevins et al., Apr. 26, 2007, “EEG Electrode Headset”), 20090088619 (Turner et al., Apr. 2, 2009, “Self-Locating Sensor Mounting Apparatus”), 20110098593 (Low et al., Apr. 28, 2011, “Head Harness & Wireless EEG Monitoring System”), 20110237971 (Pradeep et al., Sep. 29, 2011, “Discrete Choice Modeling Using Neuro-Response Data”), 20110282231 (Pradeep et al., Nov. 17, 2011, “Mechanisms for Collecting Electroencephalography Data”), 20110282232 (Pradeep et al., Nov. 17, 2011, “Neuro-Response Data Synchronization”), 20120072289 (Pradeep et al., Mar. 22, 2012, “Biometric Aware Content Presentation”), and 20130185144 (Pradeep et al., Jul. 18, 2013, “Systems and Methods for Analyzing Neuro-Response Data and Virtual Reality Environments”).

8. Device with Multiple Arms Radially-Forward from Rear and EEG/Brainwave Sensor(s)

Devices in this category hold electromagnetic brain activity sensors in contact with (or proximity to) a person's head using multiple sensor-holding protrusions, fingers, or arms which extend radially forward from a central position at the rear of a person's head. In an example, radially-extending protrusions, fingers, or arms can curve around the head toward the top and sides of the head. To use the colorful language from the previous category, now a figurative starfish (or octopus) is clinging to the back of the person's head. Such devices can be less obtrusive than some of those in the preceding categories, but can still attract attention if worn during the activities of daily life.

Prior art which appears to be within this category includes U.S. Pat. No. 4,770,180 (Schmidt et al., Sep. 13, 1988, “Electroencephalographic Head Set with a Disposable Monitor”), U.S. Pat. No. 4,967,038 (Gevins et al., Oct. 30, 1990, “Dry Electrode Brain Wave Recording System”), U.S. Pat. No. 5,038,782 (Gevins et al., Aug. 13, 1991, “Electrode System for Brain Wave Detection”), and U.S. Pat. No. D565,735 (Washbon, Apr. 1, 2008, “Electrode Headset”); and U.S. patent applications 20070225585 (Washbon and Delic, Sep. 27, 2007, “Headset for Electrodes”), 20070238945 (Delic et al., Oct. 11, 2007, “Electrode Headset”), 20090105576 (Do et al., Apr. 23, 2009, “Electrode Conductive Element”), 20120029379 (Sivadas, Feb. 2, 2012, “Mind Strength Trainer”), and 20130046206 (Preminger, Feb. 21, 2013, “System and Method for Neurocognitive Training and/or Neuropsychological Assessment”).

9. Device with Multiple Arms Radially-Backward from Front and EEG/Brainwave Sensor(s)

Devices in this category hold electromagnetic brain activity sensors in contact with (or proximity to) a person's head using multiple sensor-holding protrusions, fingers, or arms which extend radially backward from a position on the front of a person's head (such as the forehead). In an example, radially-extending protrusions, fingers, or arms can curve around the head toward the top and sides of the head. Such devices can be obtrusive and attract attention, especially if worn to a showing of the movie “Aliens”. Prior art which appears to be within this category includes U.S. patent application 20020188216 (Kayyali et al., Dec. 12, 2002, “Head Mounted Medical Device”).

10. Device with Circular Horizontal Loop (e.g. Headband Style) and EEG/Brainwave Sensor(s)

Devices in this category hold electromagnetic brain activity sensors in contact with (or proximity to) a person's head using a sensor-positioning member which is configured like a headband, ring, or other generally-circular member which encircles a person's head in (or close to) a horizontal plane when the person is upright. In an example, such a device can span a portion of a person's forehead as it encircles the person's head. Since devices in this category can span a portion of the forehead, such devices can be used with sensors which require contact with (or proximity to) portions of the head which do not have hair. Such devices can be appropriate for wearing while running or doing other types of exercise, but there are still many settings wherein wearing a headband or head-encircling ring is generally not appropriate.

Prior art which appears to be within this category includes U.S. Pat. No. 6,001,065 (Devito, Dec. 14, 1999, “Method and Apparatus for Measuring and Analyzing Physiological Signals for Active or Passive Control of Physical and Virtual Spaces and the Contents Therein”), U.S. Pat. No. 6,171,258 (Karakasoglu et al., Jan. 9, 2001, “Multi-Channel Self-Contained Apparatus and Method for Diagnosis of Sleep Disorders”), U.S. Pat. No. 6,254,536 (Devito, Jul. 3, 2001, “Method and Apparatus for Measuring and Analyzing Physiological Signals for Active or Passive Control of Physical and Virtual Spaces and the Contents Therein”), U.S. Pat. No. 6,811,538 (Westbrook et al., Nov. 2, 2004, “Sleep Apnea Risk Evaluation”), U.S. Pat. No. 7,297,119 (Westbrook et al., Nov. 20, 2007, “Sleep Apnea Risk Evaluation”), and U.S. Pat. No. 7,885,706 (Ludvig et al., Feb. 8, 2011, “System and Device for Seizure Detection”).

Prior art which appears to be within this category also includes U.S. patent applications: 20010056225 (DeVito, Dec. 27, 2001, “Method and Apparatus for Measuring and Analyzing Physiological Signals for Active or Passive Control of Physical and Virtual Spaces and the Contents Therein”), 20020165462 (Westbrook et al., Nov. 7, 2002, “Sleep Apnea Risk Evaluation”), 20020188216 (Kayyali et al., Dec. 12, 2002, “Head Mounted Medical Device”), 20040267152 (Pineda, Dec. 20, 2004, “Method and System for Predicting and Preventing Seizures”), 20050027207 (Westbrook et al., Feb. 3, 2005, “Sleep Apnea Risk Evaluation”), and 20070249952 (Rubin et al., Oct. 25, 2007, “Systems and Methods for Sleep Monitoring”).

Prior art which appears to be within this category also includes U.S. patent applications: 20080082019 (Ludving et al., Apr. 3, 2008, “System and Device for Seizure Detection”), 20090281446 (Ludvig et al., Nov. 12, 2009, “System and Device for Seizure Detection”), 20100099954 (Dickinson et al., Apr. 22, 2010, “Data-Driven Sleep Coaching System”), 20120150545 (Simon, Jun. 14, 2012, “Brain-Computer Interface Test Battery for the Physiological Assessment of Nervous System Health”), 20130060097 (Rubin, Mar. 7, 2013, “Multi-Modal Sleep System”), 20130127708 (Jung et al., May 23, 2013, “Cell-Phone Based Wireless and Mobile Brain-Machine Interface”), and 20130338446 (Van Vugt et al., Dec. 19, 2013, “Sleep Disturbance Monitoring Apparatus”).

11. Device with Top Semicircular Loop (e.g. Headphone Style) and EEG/Brainwave Sensor(s)

Devices in this category hold electromagnetic brain activity sensors in contact with (or proximity to) a person's head using a (semicircular) arcuate member which looks like a set of headphones, hair band, or tiara. In an example, such a device can loop over the top of a person's head, from one side to the other side. In an example, such a device can loop over the top of a person's head from one ear to the other ear. In example, such a device can not only look like a set of headphones, but can actually be a set of headphones, wherein these headphones also include one or more electromagnetic brain activity sensors. Wearing a set of headphones or a hair band is more common (and thus may attract less attention) than wearing most of the devices discussed in preceding categories, but there are still many settings wherein wearing such a device would attract attention and be inappropriate.

Prior art which appears to be within this category includes U.S. Pat. No. 4,697,598 (Bernard et al., Oct. 6, 1987, “Evoked Potential Autorefractometry System”), U.S. Pat. No. 4,709,702 (Sherwin, Dec. 1, 1987, “Electroencephalographic Cap”), U.S. Pat. No. 5,740,812 (Cowan, Apr. 21, 1998, “Apparatus for and Method of Providing Brainwave Biofeedback”), U.S. Pat. No. 6,154,669 (Hunter et al., Nov. 28, 2000, “Headset for EEG Measurements”), U.S. Pat. No. 6,167,298 (Levin, Dec. 26, 2000, “Devices and Methods for Maintaining an Alert State of Consciousness Through Brain Wave Monitoring”), U.S. Pat. No. 7,689,274 (Mullen et al., Mar. 30, 2010, “Brain-Wave Aware Sleep Management”), U.S. Pat. No. 8,271,075 (Chuang et al., Sep. 18, 2012, “Audio Headset with Bio-Signal Sensors”), and U.S. Pat. No. 8,301,218 (Nguyen et al., Oct. 30, 2012, “Contoured Electrode”), U.S. Pat. No. 8,812,075 (Nguyen et al., Aug. 19, 2014, “Contoured Electrode”).

Prior art which appears to be within this category also includes U.S. patent applications: 20120029379 (Sivadas, Feb. 2, 2012, “Mind Strength Trainer”), 20120226127 (Asjes et al., Sep. 6, 2012, “Device for Positioning Electrodes on a User's Scalp”), 20130177883 (Barnehama et al., Jul. 11, 2013, “Systems and Methods for Directing Brain Activity”), and 20130310676 (Jung, Nov. 21, 2013, “EEG Hair Band”).

12. Device with Rear Semicircular Loop and EEG/Brainwave Sensor(s)

Devices in this category hold electromagnetic brain activity sensors in contact with (or proximity to) a person's head using a (semicircular) arcuate member which loops around the rear portion of a person's head, from one side to the other side. In an example, such a device can loop around the rear portion of a person's head from one ear to the other ear. Such a device can be less obtrusive than many of the devices in preceding categories because it does not span the top of the head or face, but it is not well-suited for use with sensors which require contact with skin without hair. Prior art which appears to be within this category includes U.S. patent application 20140316230 (Denison et al., Oct. 23, 2014, “Methods and Devices for Brain Activity Monitoring Supporting Mental State Development and Training”).

13. Device with Frontal Semicircular Loop and EEG/Brainwave Sensor(s)

Devices in this category hold electromagnetic brain activity sensors in contact with (or proximity to) a person's head using a (semicircular) arcuate member which loops around the front of a person's head, from one side to the other side. In an example, such a device can loop around the front of a person's head from one ear to the other ear. In an example, such a device can span a person's forehead. Such a device can be well-suited for use with sensors which require contact with skin without hair, but can be somewhat obtrusive since it spans a portion of a person's face. Prior art which appears to be within this category includes U.S. patent application 20080177197 (Lee et al., Jul. 24, 2008, “Method and Apparatus for Quantitatively Evaluating Mental States Based on Brain Wave Signal Processing System”).

14. Device Like Eyeglasses or Other Eyewear with EEG/Brainwave Sensor(s)

Devices in this category hold electromagnetic brain activity sensors in contact with (or proximity to) a person's head using a sensor-positioning member which looks like a pair of eyeglasses, goggles, or other eyewear. In an example, such a device can span from one ear, to the face, across the face (over the bridge of the nose), and then to the other ear. In example, such a device can not only look like a pair of eyeglasses, but can actually be a pair of eyeglasses, wherein these eyeglasses include one or more electromagnetic brain activity sensors. Some of the art in this category predominantly focuses on the optical aspects of a pair of eyeglasses, with only tangential mention of a possible EEG sensor, but such art is included in this category for the sake of completeness. Wearing a pair of eyeglasses is very common and thus attracts less attention than virtually all of the devices discussed in preceding categories. However, conventional eyeglass frames (especially those with straight side pieces) do not contact a person's temple or forehead. Accordingly, conventional eyeglass frame configurations are not ideally-suited for holding one or more electromagnetic brain activity sensors in contact with a person's temple and/or forehead.

Prior art which appears to be within this category includes U.S. Pat. No. 7,344,244 (Goodall et al., Mar. 18, 2008, “Adjustable Lens System with Neural-Based Control”), U.S. Pat. No. 7,390,088 (Goodall et al., Jun. 24, 2008, “Adjustable Lens System with Neural-Based Control”), U.S. Pat. No. 7,486,988 (Goodall et al., Feb. 3, 2009, “Method and System for Adaptive Vision Modification”), U.S. Pat. No. 8,244,342 (Goodall et al., Aug. 14, 2012, “Method and System for Adaptive Vision Modification”), U.S. Pat. No. 8,346,354 (Hyde et al., Jan. 1, 2013, “Determining a Neuromodulation Treatment Regimen in Response to Contactlessly Acquired Information”), U.S. Pat. No. 8,467,133 (Miller, Jun. 18, 2013, “See-Through Display with an Optical Assembly Including a Wedge-Shaped Illumination System”), U.S. Pat. No. 8,472,120 (Border et al., Jun. 25, 2013, “See-Through Near-Eye Display Glasses with a Small Scale Image Source”), U.S. Pat. No. 8,477,425 (Border et al., Jul. 2, 2013, “See-Through Near-Eye Display Glasses Including a Partially Reflective, Partially Transmitting Optical Element”), U.S. Pat. No. 8,482,859 (Border et al., Jul. 9, 2013, “See-Through Near-Eye Display Glasses Wherein Image Light Is Transmitted to and Reflected From an Optically Flat Film”), U.S. Pat. No. 8,488,246 (Border et al., Jul. 16, 2013, “See-Through Near-Eye Display Glasses Including a Curved Polarizing Film in the Image Source, a Partially Reflective, Partially Transmitting Optical Element and an Optically Flat Film”), and U.S. Pat. No. 8,562,540 (Goodall et al., Oct. 22, 2013, “Method and System for Adaptive Vision Modification”).

Prior art which appears to be within this category also includes U.S. patent applications: 20060252978 (Vesely et al., Nov. 9, 2006, “Biofeedback Eyewear System”), 20060252979 (Vesely et al., Nov. 9, 2006, “Biofeedback Eyewear System”), 20070010757 (Goodall et al., Jan. 11, 2007, “Method and System for Adaptive Vision Modification”), 20070019279 (Goodall et al., Jan. 25, 2007, “Adjustable Lens System with Neural-Based Control”), 20070106145 (Kim et al., May 10, 2007, “Accessories for Remote Monitoring”), 20080161673 (Goodall et al., Jul. 3, 2008, “Method and System for Adaptive Vision Modification”), 20110028798 (Hyde et al., Feb. 3, 2011, “Electronically Initiating an Administration of a Neuromodulation Treatment Regimen Chosen in Response to Contactlessly Acquired Information”), 20110029038 (Hyde et al., Feb. 3, 2011, “Determining a Neuromodulation Treatment Regimen in Response to Contactlessly Acquired Information”), 20110029044 (Hyde et al., Feb. 3, 2011, “Stimulating a Nervous System Component of a Mammal in Response to Contactlessly Acquired Information”), 20110221656 (Haddick et al., Sep. 15, 2011, “Displayed Content Vision Correction with Electrically Adjustable Lens”), and 20110221669 (Shams et al., Sep. 15, 2011, “Gesture Control in an Augmented Reality Eyepiece”).

Prior art which appears to be within this category also includes U.S. patent applications: 20110221672 (Osterhout et al., Sep. 15, 2011, “Hand-Worn Control Device in an Augmented Reality Eyepiece”), 20110222745 (Osterhout et al., Sep. 15, 2011, “Method and Apparatus for Biometric Data Capture”), 20110227820 (Haddick et al., Sep. 22, 2011, “Lock Virtual Keyboard Position in an Augmented Reality Eyepiece”), 20120062445 (Haddick et al., Mar. 15, 2012, “Adjustable Wrap Around Extendable Arm for a Head-Mounted Display”), 20120075168 (Osterhout et al., Mar. 29, 2012, “Eyepiece with Uniformly Illuminated Reflective Display”), 20120150545 (Simon, Jun. 14, 2012, “Brain-Computer Interface Test Battery for the Physiological Assessment of Nervous System Health”), 20120212398 (Border et al., 823/2012, “See-Through Near-Eye Display Glasses Including a Partially Reflective, Partially Transmitting Optical Element”), and 20120212400 (Border et al., Aug. 23, 2012, “See-Through Near-Eye Display Glasses Including a Curved Polarizing Film in the Image Source, a Partially Reflective, Partially Transmitting Optical Element and an Optically Flat Film”).

Prior art which appears to be within this category also includes U.S. patent applications: 20120218172 (Border et al., Aug. 30, 2012, “See-Through Near-Eye Display Glasses with a Small Scale Image Source”), 20120218301 (Miller, Aug. 30, 2012, “See-Through Display with an Optical Assembly Including a Wedge-Shaped Illumination System”), 20120235883 (Border et al., Sep. 20, 2012, “See-Through Near-Eye Display Glasses with a Light Transmissive Wedge Shaped Illumination System”), 20120235886 (Border et al., Sep. 20, 2012, “See-Through Near-Eye Display Glasses with a Small Scale Image Source”), 20120235887 (Border et al., Sep. 20, 2012, “See-Through Near-Eye Display Glasses Including a Partially Reflective, Partially Transmitting Optical Element and an Optically Flat Film”), and 20120235900 (Border et al., Sep. 20, 2012, “See-Through Near-Eye Display Glasses with a Fast Response Photochromic Film System for Quick Transition From Dark to Clear”).

Prior art which appears to be within this category also includes U.S. patent applications: 20120236030 (Border et al., Sep. 20, 2012, “See-Through Near-Eye Display Glasses Including a Modular Image Source”), 20120242678 (Border et al., Sep. 27, 2012, “See-Through Near-Eye Display Glasses Including an Auto-Brightness Control for the Display Brightness Based on the Brightness in the Environment”), 20120242698 (Haddick et al., Sep. 27, 2012, “See-Through Near-Eye Display Glasses with a Multi-Segment Processor-Controlled Optical Layer”), 20130056010 (Walker et al., Mar. 7, 2013, “Autonomous Positive Airway Pressure System”), 20130127980 (Haddick et al., May 23, 2013, “Video Display Modification Based on Sensor Input for a See-Through Near-to-Eye Display”), and 20130242262 (Lewis, Sep. 19, 2013, “Enhanced Optical and Perceptual Digital Eyewear”).

Prior art which appears to be within this category also includes U.S. patent applications: 20130303837 (Berka et al., Nov. 14, 2013, “Systems and Methods for Optimization of Sleep and Post-Sleep Performance”), 20130314303 (Osterhout et al., Nov. 28, 2013, “AR Glasses with User Action Control of and Between Internal and External Applications with Feedback”), 20140023999 (Greder, Jan. 23, 2014, “Detection and Feedback of Information Associated with Executive Function”), 20140267005 (Urbach, Sep. 18, 2014, “Eye Piece for Augmented and Virtual Reality”), 20140267401 (Urbach, Sep. 18, 2014, “Visual Cortex Thought Detector Interface”), 20140347265 (Aimone et al., Nov. 27, 2014, “Wearable Computing Apparatus and Method”), and 20140375545 (Ackerman et al., Dec. 25, 2014, “Adaptive Event Recognition”).

### SUMMARY OF THE INVENTION

This invention is a mobile wearable device for measuring electromagnetic brain activity. In an example, this invention can be embodied in a flexible ring and/or band (such as headband) which is worn around a person's head to measure electromagnetic brain activity. In an example, a wearable device for measuring electromagnetic brain activity can comprise a wearable ring and/or band which encircles a person's head at an anterior acute angle in the range of 0 to 45 degrees with respect to a horizontal plane (when the person's head is upright) and a plurality of electromagnetic energy sensors on the ring and/or band. In an example, this invention can be embodied in eyewear which is worn on a person's head to measure electromagnetic brain activity. In an example, eyewear can include a flexible and/or elastic rear loop which loops around the rear of a person's head to hold one or more electromagnetic energy sensors against the rear of the person's head.

In an example, this device can further comprise prongs and/or teeth which protrude into and/or under the person's hair. Such prongs and/or teeth can facilitate good electromagnetic communication with the person's head at locations which are covered by hair. In an example, prongs and/or teeth can be made from a silicone material (such as PDMS) which has been impregnated, doped, coated, or embedded with conductive material (such as a metal and/or carbon) in order to provide consistent but comfortable contact with the person's head. In an example, a metal can be silver or aluminum. In an example, carbon material can be in the form of nanotubes or graphene. In an example, electromagnetic energy sensors can be located at placement sites of Modified Combinatorial Nomenclature (MCN) electrode placement system selected from the group consisting of F3, F4, F7, F8, FPz, Fz, T3, T4, T5, T6, P3, P4, Pz, O1, O2, C3, C4, and Cz.

### INTRODUCTION TO THE FIGURES

FIG. 1 shows a device with MCN electrode locations, as background for examples which follow.

FIG. 2 shows a device with a ring and a frontal partial ring.

FIG. 3 shows a device with a front portion with a downward-facing concavity, a middle portion which curves over an ear, and a rear portion.

FIG. 4 shows a device with upper and lower diverging portions from the temple around the back of a head.

FIG. 5 shows a device with an eyewear frame with an upward arm from the sidepiece to the temple or forehead.

FIG. 6 shows a device with front and rear loops spanning from an ear over the top of a head.

FIG. 7 shows a device with a loop from an ear around a forehead and two loops around the back of a head.

FIG. 8 shows a device with an arcuate ring which rests on an ear.

FIG. 9 shows a device with a ring with prongs or teeth which protrude into hair.

FIG. 10 shows a device with an arcuate ring above an ear.

FIG. 11 shows a device with a loop over the top of a head and a loop around the front and back of an ear.

FIG. 12 shows a device with a portion from an ear to the top of a head, a portion from an ear to the back of a head, and an arcuate link between these portions.

FIG. 13 shows a device with a ring and two loops from the ring over the top of a head.

FIG. 14 shows a device with portions which curve around an ear and loop around the back of a head.

FIG. 15 shows a device with portions which curve around an ear and loop around a forehead.

FIG. 16 shows a device with portions which curve around an ear and loop over the top of a head.

FIG. 17 shows a device with a wide saddle-shaped device on top of a head.

FIG. 18 shows a device with a narrow saddle-shaped device on top of a head.

FIG. 19 shows a device with a tiara-shaped or hair-band-shaped device on top of a head.

FIG. 20 shows a device with a loop over the top of a head, a loop around the back of a head, and a front-ear-contacting portion.

FIG. 21 shows a device with a loop over the top of a head and a loop around the back of a head.

FIG. 22 shows a device with loop over the top of a head and a portion which curves around an ear.

FIG. 23 shows a device with an undulating head-circling ring which curves over the top of an ear.

FIG. 24 shows a device with a loop around the back of a head and a portion which curves around an ear.

FIG. 25 shows a device with an eyewear frame with a bifurcated sidepiece between an ear and front piece.

FIG. 26 shows a device with upper and lower loops which diverge from an ear to the back of a head.

FIG. 27 shows a device with a forehead loop, a loop over the top of a head, and a semi-circular portion which curves around an ear.

FIG. 28 shows a device with a forehead loop, a loop over the top of a head, and a quarter-circular portion which curves around an ear.

FIG. 29 shows a device with a loop over the top of a head, a loop around the back of a head, and a portion which curves around an ear.

FIG. 30 shows a device with a loop over the top of a head and a portion which encircles an ear.

FIG. 31 shows a device with an undulating ring around a head which rests on an ear.

FIG. 32 shows a device with an eyewear frame with an upward arcuate protrusion over an ear.

FIG. 33 shows a device with a loop over the upper-rear quadrant of a head and a portion which curves around an ear.

FIG. 34 shows a device with an upper front loop and an upper rear loop.

FIG. 35 shows a device with an upper rear loop, a front ear portion, and a rear ear portion.

FIG. 36 shows a device with an undulating ring around a head and also an upper rear loop.

FIG. 37 shows a device with an eyewear frame with an upward undulation on a sidepiece.

FIG. 38 shows a device with bi-level undulating ring and a loop over the top of a head.

FIG. 39 shows a device with an eyewear frame with a protrusion which extends rearward from an ear.

FIG. 40 shows a device with a ring with a concave undulation over an ear.

FIG. 41 shows a device with an undulating ring which rests on an ear.

FIG. 42 shows a device with front and rear loops from an ear over the top of a head.

FIG. 43 shows a device with a portion which loops over a head, a portion which loops around the back of a head, and an arcuate link between them.

FIG. 44 shows a device with an eyewear frame with a concave bifurcation along the sidepiece.

FIG. 45 shows a device with a front loop which spans a forehead and an upper loop over the top of a head.

FIG. 46 shows a device with an eyewear frame with an undulating sidepiece.

FIG. 47 shows a device with an undulating ring and a loop over the top of a head.

FIG. 48 shows a device with an eyewear frame with an upward and rearward protrusion from the top of an ear.

FIG. 49 shows a device with an eyewear frame with an upward loop from the top of an ear.

FIG. 50 shows a device with a tiara shaped or hairband shaped device which loops from an ear over the top of a head.

FIG. 51 shows a device with an eyewear frame with an upward and rearward loop from the top of an ear.

FIG. 52 shows a device with an angled (non-horizontal) ring and a loop around a forehead.

FIG. 53 shows a device with a loop around the back of a head and a portion which curves around an ear.

FIG. 54 shows a device with a ring around a head and two loops over the upper-rear portion of a head.

FIG. 55 shows a device with an eyewear frame with a concave sidepiece.

FIG. 56 shows a device with an eyewear frame which curves around both the front and back of an ear.

FIG. 57 shows a device with an upper branch and a lower branch which diverge as they loop from an ear around the back of a head.

FIG. 58 shows a device with an eyewear frame with a loop over the top of a head.

FIG. 59 shows a device with a loop over the frontal top of a head and a portion which encircles an ear.

FIG. 60 shows a device with an eyewear frame with a sidepiece whose mid-section curves upward.

FIG. 61 shows a device with an eyewear frame whose sidepiece has an upward protrusion from its mid-section.

FIG. 62 shows a device with an arcuate ring around a head, wherein the front portion is higher than the rear portion.

FIG. 63 shows a device with a ring around a head and a central loop over the top of a head.

FIG. 64 shows a device with two-bifurcated loops from an ear over the top of a head.

FIG. 65 shows a device with an eyewear frame with an upper-rear loop over the top of a head.

FIG. 66 shows a device with circular or elliptical ring around a head and a central loop over the top of the head.

FIG. 67 shows a device with two loops which diverge as they extend from an ear to the top and the back of a head.

FIG. 68 shows a device with an eyewear frame with a sidepiece with an upward arm on its mid-section.

FIG. 69 shows a device with a first device with an undulating loop spanning from an ear over the top of a head.

FIG. 70 shows a device with a second device with an undulating loop spanning from an ear over the top of a head.

FIG. 71 shows a device with a third device with an undulating loop spanning from an ear over the top of a head.

FIG. 72 shows a device with a fourth device with an undulating loop spanning from an ear over the top of a head.

FIG. 73 shows a device with flexible prongs and/or teeth which slide into and/or under a hair.

FIG. 74 shows a device with an eyewear frame with flexible prongs and/or teeth which slide into and/or under a hair.

FIG. 75 shows a device with a first device with a ring and an ear-contacting and/or ear-covering portion.

FIG. 76 shows a device with a second device with a ring and an ear-contacting and/or ear-covering portion.

### DETAILED DESCRIPTION OF THE FIGURES

Disclosed herein is a mobile wearable device with electromagnetic energy sensors which is worn on a person's head and collects data concerning the person's electromagnetic brain activity. Such a mobile wearable device has numerous potential applications in the fields of: medicine, health, and fitness; communication and telepresence; entertainment, augmented reality (AR), virtual reality (VR), and gaming; sports; navigation; telerobotics; and defense. For example, such a mobile wearable device can serve as a method of communication by people who are paralyzed or have otherwise lost muscle control. As another example, such a mobile wearable device can be used as a brain to computer interface (BCI) in situations where other forms of human-to-computer interaction (such as a touch-based interface or a speech-based interface) are undesirable, difficult, or even impossible.

A mobile wearable device for measuring brain activity can be specified based on the geometry of the device, including the configurations/shapes of different arms/portions of the device which span the person's head. Additionally, a mobile wearable device for measuring brain activity can be specified based on the locations of electromagnetic energy sensors which are held on the person's head. In an example, electromagnetic energy sensors can be EEG sensors. Sensor locations can be specified using the electrode locations of the internationally-recognized Modified Combinatorial Nomenclature (MCN) electrode placement system. Electromagnetic energy sensors can be located at one or more locations selected from the group of MCN electrode sites consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, one or more reference electrodes can also be located at the A1 and/or A2 locations.

In an example, a mobile wearable device for measuring electromagnetic brain activity can be embodied in a soft ring and/or band (such as headband) which is worn on a person's head. In an example, a wearable device for measuring electromagnetic brain activity can comprise a wearable ring and/or band which encircles a person's head at an anterior acute angle in the range of 0 to 45 degrees with respect to a horizontal plane (when the person's head is upright) and a plurality of electromagnetic energy sensors on the ring and/or band. In an example, a mobile wearable device for measuring electromagnetic brain activity can be embodied in eyewear which is worn on a person's head. In an example, eyewear can have a flexible and/or elastic rear loop which loops around the rear of a person's head.

In an example, electromagnetic energy sensors of this device can be located at placement sites of Modified Combinatorial Nomenclature (MCN) electrode placement system which are selected from the group consisting of F3, F4, F7, F8, Fz, T3, T4, T5, T6, P3, P4, Pz, O1, O2, C3, C4, and Cz. The electromagnetic energy sensors can be made from a silicone material (such as PDMS) which has been impregnated, doped, coated, or embedded with conductive material (such as metal and/or carbon) in order to provide consistent but comfortable contact with the person's head. In an example, this device can further comprise prongs and/or teeth which protrude from the ring and/or band into and/or under the person's hair. In an example, the prongs and/or teeth can be made from a silicone material (such as PDMS) which has been impregnated, doped, coated, or embedded with conductive material (such as metal and/or carbon) in order to provide consistent but comfortable contact with the person's head.

In an example, a wearable device for measuring electromagnetic brain activity can be embodied in eyewear which is worn on a person's head to measure electromagnetic brain activity. In an example, the eyewear can include a flexible and/or elastic rear loop which loops around the rear of a person's head to hold one or more electromagnetic energy sensors against the rear of the person's head. In an example, this device can further comprise prongs and/or teeth which protrude into and/or under the person's hair. This can enable good electromagnetic communication with the person's head at locations on the person's head which are covered by hair. In an example, the prongs and/or teeth can be made from a silicone material (such as PDMS) which has been impregnated, doped, coated, or embedded with conductive material (such as a metal and/or carbon) in order to provide consistent but comfortable contact with the person's head. In an example, conductive metal can be silver or aluminum. In an example, carbon material can be in the form of nanotubes or graphene. In an example, electromagnetic energy sensors can be located at placement sites of Modified Combinatorial Nomenclature (MCN) electrode placement system which are selected from the group consisting of F3, F4, F7, F8, FPz, Fz, T3, T4, T5, T6, P3, P4, Pz, O1, O2, C3, C4, and Cz.

In an example, a wearable device for measuring electromagnetic brain activity can comprise: a wearable ring and/or band which is configured to encircle a person's head at an anterior acute angle in the range of 0 to 45 degrees with respect to a horizontal plane when the person's head is upright, wherein the ring and/or band has prongs and/or teeth which protrude into and/or under the person's hair; and a plurality of electromagnetic energy sensors on the band; wherein one or more of the electromagnetic energy sensors are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; and wherein one or more of the electromagnetic energy sensors are made from an elastic polymer.

In an example, a wearable device for measuring electromagnetic brain activity can comprise: a wearable ring and/or band which is configured to encircle a person's head at an anterior acute angle in the range of 0 to 45 degrees with respect to a horizontal plane when the person's head is upright; and a plurality of electromagnetic energy sensors on the band; wherein one or more of the electromagnetic energy sensors are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; wherein one or more of the electromagnetic energy sensors are made from a polymer which has been impregnated, doped, coated, or embedded with conductive material; and wherein one or more of the electromagnetic energy sensors have prongs and/or teeth which protrude into and/or under the person's hair.

In an example, a wearable device for measuring electromagnetic brain activity can comprise: a wearable ring and/or band which is configured to encircle a person's head at an anterior acute angle in the range of 0 to 45 degrees with respect to a horizontal plane when the person's head is upright; and a plurality of electromagnetic energy sensors on the band; wherein one or more of the electromagnetic energy sensors are located at placement sites of Modified Combinatorial Nomenclature (MCN) electrode placement system selected from the group consisting of O1, Oz, FP1, FPz, PO7, AF7, P7, F7, TP7, FT7, and T7; wherein one or more of the electromagnetic energy sensors are made from a silicone material which has been impregnated, doped, coated, or embedded with silver or carbon; and wherein one or more of the electromagnetic energy sensors have prongs and/or teeth which protrude into and/or under the person's hair.

In an example, an electromagnetic energy sensor can be a capacitive electromagnetic energy sensor. In an example, an electromagnetic energy sensor can be a dry electromagnetic energy sensor. In an example, an electromagnetic energy sensor can be made with a low-conductivity material which has been doped, impregnated, or coated with a high-conductivity material. In an example, an electromagnetic energy sensor can comprise a dielectric layer of low-conductivity material between two layers of high-conductivity material. In an example, an electromagnetic energy sensor can comprise a layer of high-conductivity material between two layers of low-conductivity material. In an example, an electromagnetic energy sensor can comprise low-conductivity fibers and highly-conductive fibers which are braided or woven together.

In an example, an electromagnetic energy sensor can be made by printing high-conductivity ink onto a low-conductivity textile or fabric. In an example, an electromagnetic energy sensor can be made by printing a conductive elastomeric material onto a low-conductivity textile or fabric. In an example, an electromagnetic energy sensor can be made by melting or adhering elastomeric conductive material onto a low-conductivity textile or fabric. In an example, an electromagnetic energy sensor can be made by embroidering conductive material onto a low-conductivity textile or fabric. In an example, an electromagnetic energy sensor can be made by weaving, knitting, sewing, embroidering, layering, laminating, adhering, melting, fusing, printing, spraying, painting, or pressing electroconductive material into (or onto) a fabric or textile. In an example, electroconductive threads, fibers, yarns, strands, filaments, traces, and/or layers within a fabric or textile can be configured near a person's skin in order to receive electromagnetic energy.

In an example, an electromagnetic energy sensor can be attached to a wearable device for measuring electromagnetic brain activity using an attachment mechanism selected from the group consisting of: adhesive, band, buckle, button, channel, clasp, clip, electronic connector, flexible channel, hook, hook-and-eye mechanism, magnet, pin, plug, pocket, rivet, sewing, snap, tape, tie, and zipper. In an example, an electromagnetic energy sensor can be attached to a wearable device for measuring electromagnetic brain activity by printing, laminating, adhering, embroidering, melting, and/or sewing electroconductive material. In an example, an electronically-functional fabric or textile with electromagnetic energy sensors can be created by weaving, knitting, sewing, embroidering, layering, laminating, adhering, melting, fusing, printing, spraying, painting, or pressing together electroconductive threads, fibers, yarns, strands, filaments, traces, and/or layers. In an example, electroconductive threads, yarns, fibers, strands, channels, and/or traces comprising electromagnetic energy sensors can have shapes or configurations which are selected from the group consisting of: circular, elliptical, or other conic section; square, rectangular, hexagon, or other polygon; parallel; perpendicular; crisscrossed; nested; concentric; sinusoidal; undulating; zigzagged; and radial spokes.

In an example, an electronically-functional fabric or textile with electromagnetic energy sensors can be created by printing, spraying, or otherwise depositing electroconductive ink or resin onto an otherwise non-conductive fabric or textile. In an example, an electronically-functional circuit with electromagnetic energy sensors can be created as part of a wearable device for measuring electromagnetic brain activity by printing a conductive pattern with electroconductive ink or resin. In an example, an electronically-functional fabric or textile with electromagnetic energy sensors can be created by laminating electro-conductive members onto a non-conductive substrate. In an example, an electronically-functional fabric or textile with electromagnetic energy sensors can be created by embroidering a generally non-conductive fabric or textile member with electro-conductive members. In an example, an electronically-functional circuit with electromagnetic energy sensors can be created for a wearable device for measuring electromagnetic brain activity by embroidering a conductive pattern with electroconductive thread.

In an example, an electromagnetic energy sensor can be made with a low-conductivity material selected from the group consisting of: acetate, acrylic, cotton, denim, elastane, latex, linen, Lycra™, neoprene, nylon, nylon, polyester, wool, silicone, polydimethylsiloxane (PDMS), silk, spandex, and rayon. In an example, an electromagnetic energy sensor can be made with a high-conductivity material selected from the group consisting of: aluminum or aluminum alloy; carbon nanotubes, graphene, or other carbon-based material; copper or copper alloy; gold; nickel; silver; and steel. In an example, an electromagnetic energy sensor can be made with polydimethylsiloxane (PDMS) which has been doped or impregnated with aluminum, carbon (in one or more various configurations and formulations), copper, gold, nickel, silver, or steel. In an example, an electromagnetic energy sensor can be made from polydimethylsiloxane (PDMS) and carbon nanotubes. In an example, an electromagnetic energy sensor can be made from polydimethylsiloxane (PDMS) and silver.

In an example, a mobile wearable device for measuring electromagnetic brain activity can comprise: a flexible arcuate ring and/or band which is configured to be worn around a person's head, wherein a front portion of the ring and/or band loops around the person's forehead, and wherein a rear portion of the ring and/or band loops around the rear of the person's head; a plurality of electromagnetic energy sensors which measure electromagnetic brain activity, wherein a first subset of the electromagnetic energy sensors are located on the front portion of the ring or band, and wherein a second subset of the electromagnetic energy sensors are located on the rear portion of the ring or band; and a plurality of prongs and/or teeth which protrude into and/or under the person's hair. In an example, the prongs and/or teeth can facilitate electromagnetic communication between electromagnetic energy sensors in the second subset of electromagnetic energy sensors and the person's brain.

In an example, a mobile wearable device for measuring electromagnetic brain activity can comprise: a flexible arcuate ring and/or band which is configured to be worn around a person's head, wherein a front portion of the ring and/or band loops around the person's forehead, and wherein a rear portion of the ring and/or band loops around the rear of the person's head; a flexible loop which loops over the top of the person's head; a plurality of electromagnetic energy sensors which measure electromagnetic brain activity, wherein a first subset of the electromagnetic energy sensors are located on the front portion of the ring or band, wherein a second subset of the electromagnetic energy sensors are located on the rear portion of the ring or band, and wherein a third subset of the electromagnetic energy sensors are located on the flexible loop; and a plurality of prongs and/or teeth which protrude into and/or under the person's hair. In an example, the prongs and/or teeth can facilitate electromagnetic communication between electromagnetic energy sensors in the second and third subsets of electromagnetic energy sensors and the person's brain.

In an example, a mobile wearable device for measuring electromagnetic brain activity can comprise: a flexible arcuate ring and/or band which is configured to be worn around a person's head, wherein a front portion of the ring and/or band loops around the person's forehead, and wherein a rear portion of the ring and/or band loops around the rear of the person's head; an ear portion which curves around (the front and/or rear of) the person's ear; a plurality of electromagnetic energy sensors which measure electromagnetic brain activity, wherein a first subset of the electromagnetic energy sensors are located on the front portion of the ring or band, wherein a second subset of the electromagnetic energy sensors are located on the rear portion of the ring or band, and wherein a third subset of the electromagnetic energy sensors are located on the ear portion; and a plurality of prongs and/or teeth which protrude into and/or under the person's hair. In an example, the prongs and/or teeth facilitate electromagnetic communication between electromagnetic energy sensors in the second and third subsets of electromagnetic energy sensors and the person's brain.

In an example, prongs and/or teeth can be made from an elastomeric polymer which is doped, embedded, impregnated, or coated with conductive material. In an example, portions of electromagnetic energy sensors can be made from an elastomeric polymer which is doped, embedded, impregnated, or coated with conductive material. In an example, the polymer can be a silicone material. In an example, the polymer can be PDMS. In an example, the conductive material can be silver. In an example, the conductive material can be carbon. In an example, the conductive material can be carbon nanotubes or graphene.

FIG. 1 shows Modified Combinatorial Nomenclature (MCN) electrode locations on the left side of a person's head. In an example, one or more electromagnetic energy sensors (such as EEG sensors) can be located at one or more of these electrode locations. The figures and examples which follow in this disclosure show the left side of a person's head, but the devices which are specified in these examples can also be laterally symmetric on the right side of the person's head even though it is not shown.

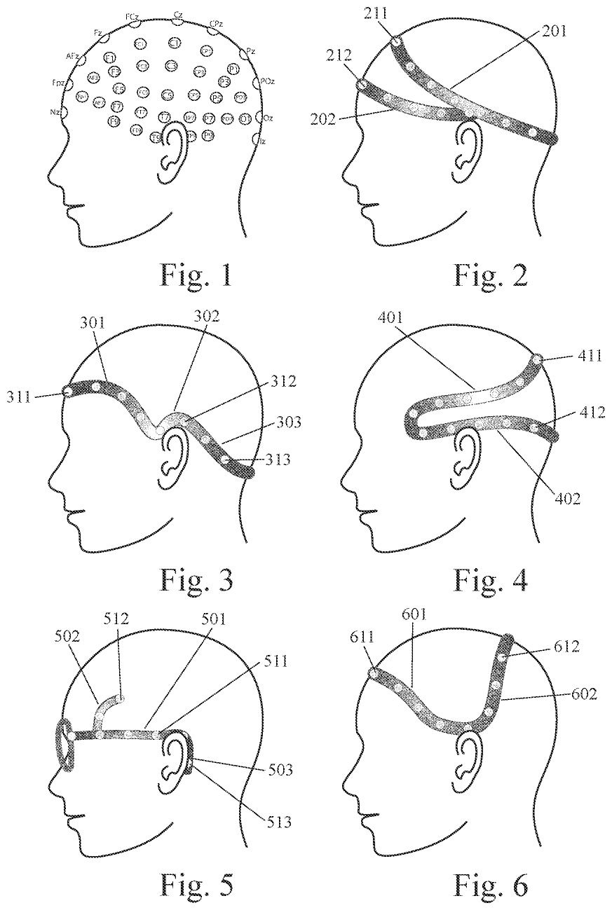

FIG. 2 shows the left side of a wearable device for measuring electromagnetic brain activity comprising: a ring 201 which encircles a person's head spanning the front and the back of the head and holds at least one first electromagnetic energy sensor 211 (substantially located at an electrode position selected from the group consisting of Iz, PO7, P7, F5, F3, F1, Fz, TP7, FC5, and C5); and a partial ring 202 which partially encircles the person's head (spanning the front of the person's head) and holds at least one second electromagnetic energy sensor 212 (substantially located at an electrode position selected from the group consisting of FP1, FPz, AF7, F7, FT7, and T7).

In an example, a wearable device for measuring electromagnetic brain activity can comprise: a ring which encircles a person's head spanning the front and the back of the head and holds at least one first electromagnetic energy sensor (substantially located at an electrode position selected from the group consisting of Iz, PO7, P7, F5, F3, F1, Fz, TP7, FC5, and C5); and a partial ring which partially encircles the person's head (spanning the front of the head).

In an example, a wearable device for measuring electromagnetic brain activity can comprise: a ring which encircles a person's head spanning the front and the back of the person's head; and a partial ring which partially encircles the person's head (spanning the front of the head) and holds at least one second electromagnetic energy sensor (substantially located at an electrode position selected from the group consisting of FP1, FPz, AF7, F7, FT7, and T7).

In an example: a first virtual plane which best fits the ring can intersect a horizontal plane when the person is standing up, forming a first forward-facing angle between 5 and 75 degrees; a second virtual plane which best fits the partial ring can intersect the horizontal plane, forming a second forward-facing angle between 5 and 75 degrees; and the first angle can be at least 5 degrees greater than the second angle. In an example, the intersection of the ring and the partial ring can form a forward-facing angle between 5 and 65 degrees. In an example, the front of the ring can be higher than the rear of the ring. In an example, a frontal portion of the partial ring can be lower than the ring. In an example, the partial ring can span the person's forehead. In an example, the partial ring can intersect and/or join the ring: at a location (directly) above the person's ear; and/or at a location within 2″ of the front-to-back midpoint of the side of the person's head. In an example, the ring and/or the partial ring can have an (upward-facing) concavity. In an example, the ring can be more upwardly-concave than the partial ring. In an example, the ring can be less upwardly-concave than partial ring. In an example, the ring can be circular, oval, or elliptical. In an example, the partial ring can be a semicircle, a semi-oval, or a semi-ellipse. In an example, this device can be a bifurcating ring around a person's head, wherein the ring bifurcates as it spans the front of the person's head. In an example, this device can comprise a headband.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 3 shows the left side of a wearable device for measuring electromagnetic brain activity comprising: a front portion 301 which spans from a person's ear to their forehead and holds at least one first electromagnetic energy sensor 311 (substantially located at an electrode position selected from the group consisting of FPz, AF3, F5, and FT7), wherein the front portion has a downward-facing concavity; a middle portion 302 which is connected to the front portion, curves over the top of the person's outer ear, and holds at least one second electromagnetic energy sensor 312 (substantially located at an electrode position selected from the group consisting of TP7, T9, and T7); and a rear portion 303 which is connected to the middle portion, spans the rear of the person's head, and holds at least one third electromagnetic energy sensor 313 (substantially located at an electrode position selected from the group consisting of P9 and TP9).

In an example, the left side of a wearable device for measuring electromagnetic brain activity can comprise: a front portion which spans from a person's ear to their forehead and holds at least one first electromagnetic energy sensor (substantially located at an electrode position selected from the group consisting of FPz, AF3, F5, and FT7), wherein the front portion has a downward-facing concavity; a middle portion which is connected to the front portion and curves over the top of the person's outer ear; and a rear portion which is connected to the middle portion and spans the rear of the person's head.

In an example, the left side of a wearable device for measuring electromagnetic brain activity can comprise: a front portion which spans from a person's ear to their forehead and has a downward-facing concavity; a middle portion which is connected to the front portion, curves over the top of the person's outer ear, and holds at least one second electromagnetic energy sensor (substantially located at an electrode position selected from the group consisting of TP7, T9, and T7); a rear portion which is connected to the middle portion and spans the rear of the person's head.

In an example, the left side of a wearable device for measuring electromagnetic brain activity can comprise: a front portion which spans from a person's ear to their forehead and has a downward-facing concavity; a middle portion which is connected to the front portion and curves over the top of the person's outer ear; a rear portion which is connected to the middle portion, spans the rear of the person's head, and holds at least one third electromagnetic energy sensor (substantially located at an electrode position selected from the group consisting of P9 and TP9).

In an example, this device can comprise an undulating ring which encircles a person's head, spanning their forehead and the rear of their head, with upward undulations over the tops of the person's ears. In an example, this device can comprise an undulating ring which encircles a person's head, spanning their forehead and the rear of their head, with sinusoidal waves over the tops of the person's ears. In an example, a virtual plane which best fits this undulating ring can intersect a horizontal plane when the person is standing up, forming a forward-facing angle between 5 and 75 degrees. In an example, the front of this undulating ring can be higher than the rear of this undulating ring. In an example, the rear of the undulating ring can be lower than the top of the person's ear. In an example, this device can comprise a headband.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 4 shows the left side of a wearable device for measuring electromagnetic brain activity comprising: an upper arm/portion 401 which spans from an area within 2″ of a person's temple to the back of their head and holds at least one first electromagnetic energy sensor 411 (substantially located at an electrode position selected from the group consisting of P3, P1, Pz, F9, F7, CP5, FC5, and C5); and a lower arm/portion 402 which spans from the area within 2″ of a person's temple to the back of their head and holds at least one second electromagnetic energy sensor 412 (substantially located at an electrode position selected from the group consisting of Iz, O1, Oz, PO7, P7, F9, TP7, FT9, and T7), wherein the front of the upper arm/portion is connected to the front of the lower arm/portion, and wherein the rear of the upper arm/portion is higher than the rear of the lower arm/portion.

In an example, the left side of a wearable device for measuring electromagnetic brain activity can comprise: an upper arm/portion which spans from an area within 2″ of a person's temple to the back of their head and holds at least one first electromagnetic energy sensor (substantially located at an electrode position selected from the group consisting of P3, P1, Pz, F9, F7, CP5, FC5, and C5); and a lower arm/portion which spans from the area within 2″ of a person's temple to the back of their head, wherein the front of the upper arm/portion is connected to the front of the lower arm/portion, and wherein the rear of the upper arm/portion is higher than the rear of the lower arm/portion.

In an example, the left side of a wearable device for measuring electromagnetic brain activity can comprise: an upper arm/portion which spans from an area within 2″ of a person's temple to the back of their head; and a lower arm/portion which spans from the area within 2″ of a person's temple to the back of their head and holds at least one second electromagnetic energy sensor (substantially located at an electrode position selected from the group consisting of Iz, O1, Oz, PO7, P7, F9, TP7, FT9, and T7), wherein the front of the upper arm/portion is connected to the front of the lower arm/portion, and wherein the rear of the upper arm/portion is higher than the rear of the lower arm/portion.

In an example, the upper arm/portion can have an upward-facing concavity and the lower arm/portion can have a downward-facing concavity. In an example, the device can span approximately two-thirds of the front-to-back width of the side of the person's head. In an example, the lower arms/portions of the device can rest on top the person's ears. In an example, the device overall can have a rear-facing concavity. In an example, the left-side-portion of the device can have the shape of a rear-opening “bobby pin.”

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 5 shows the left side of a wearable device for measuring electromagnetic brain activity comprising: a relatively-conventional eyeglass frame 501 which holds at least one first electromagnetic energy sensor 511 (substantially located at an electrode position selected from the group consisting of Nz, F9, FT9, FT7, and T7); an arcuate upward arm 502 which extends upward from the side of the eyeglass frame to the person's temple and/or forehead and holds at least one second electromagnetic energy sensor 512 (substantially located at an electrode position selected from the group consisting of AF7 and F5); and a rear arm 503 which curves around the rear of the person's ear and holds at least one third electromagnetic energy sensor 513 (substantially located at an electrode position selected from the group consisting of TP9 and TP7).

In an example, a wearable device for measuring electromagnetic brain activity can comprise: a relatively-conventional eyeglass frame which holds at least one first electromagnetic energy sensor (substantially located at an electrode position selected from the group consisting of Nz, F9, FT9, FT7, and T7); an arcuate upward arm which extends upward from the side of the eyeglass frame to the person's temple and/or forehead; and a rear arm which curves around the rear of the person's ear.

In an example, a wearable device for measuring electromagnetic brain activity can comprise: a relatively-conventional eyeglass frame; an arcuate upward arm which extends upward from the side of the eyeglass frame to the person's temple and/or forehead and holds at least one second electromagnetic energy sensor (substantially located at an electrode position selected from the group consisting of AF7 and F5); and a rear arm which curves around the rear of the person's ear.

In an example, a wearable device for measuring electromagnetic brain activity can comprise: a relatively-conventional eyeglass frame; an arcuate upward arm which extends upward from the side of the eyeglass frame to the person's temple and/or forehead; and a rear arm which curves around the rear of the person's ear and holds at least one third electromagnetic energy sensor (substantially located at an electrode position selected from the group consisting of TP9 and TP7).

In an example, the arcuate upward arm can have a rear-facing concavity. In an example, the arcuate upward arm can have a forward-facing concavity. In an example, an upward arm can pivot and/or rotate around its connection to the eyeglass frame. In an example, an upward arm can pivot from a first configuration in which it is aligned with and/or hidden behind the eyeglass frame when not in use and a second configuration in which is extends upward from the eyeglass frame for use.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 6 shows the left side of a wearable device for measuring electromagnetic brain activity comprising: a front arm/portion 601 which spans from an area (directly) above a person's ear to the front of the person's head and holds at least one first electromagnetic energy sensor 611 (substantially located at an electrode position selected from the group consisting of AF3, AFz, F5, FT7, and T7); and a rear arm/portion 602 which spans from the area above the person's ear to the rear of the person's head and holds at least one second electromagnetic energy sensor 612 (substantially located at an electrode position selected from the group consisting of TP7, CP5, CP3, CP1, and CPz).

In an example, the left side of a wearable device for measuring electromagnetic brain activity can comprise: a front arm/portion which spans from an area above a person's ear around the front of the person's head and holds at least one first electromagnetic energy sensor (substantially located at an electrode position selected from the group consisting of AF3, AFz, F5, FT7, and T7); and a rear arm/portion which spans from the area above the person's ear around the rear of the person's head.

In an example, the left side of a wearable device for measuring electromagnetic brain activity can comprise: a front arm/portion which spans from an area above a person's ear around the front of the person's head; and a rear arm/portion which spans from the area above the person's ear around the rear of the person's head and holds at least one second electromagnetic energy sensor (substantially located at an electrode position selected from the group consisting of TP7, CP5, CP3, CP1, and CPz).

In an example, the device can be shaped like a saddle on top of the person's head. In an example, the front arm/portion and the rear arm/portion can connect at an area which is directly over the person's ear. In an example, the device can rest on top of the person's ears. In an example, the rear arm/portion can be more vertical than the front arm/portion. In an example, the front arm/portion can span the person's forehead. In an example, the front arm/portion can have a downward-facing concavity.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

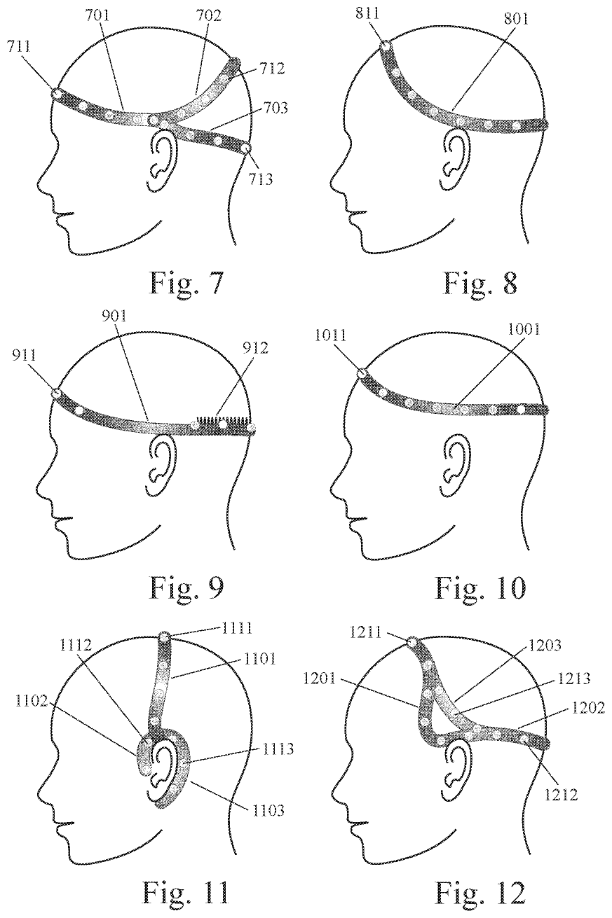

FIG. 7 shows the left side of a wearable device for measuring electromagnetic brain activity comprising: a first arm/portion 701 which spans from an area (directly) above a person's ear to the person's forehead and holds at least one first electromagnetic energy sensor 711 (substantially located at an electrode position selected from the group consisting of FP1, FPz, AF7, F7, FT7, and T7); a second arm/portion 702 which spans from the area above the person's ear to the rear of the person's head and holds at least one second electromagnetic energy sensor 712 (substantially located at an electrode position selected from the group consisting of P5, P3, P1, Pz, CP5, T7); and a third arm/portion 703 which spans from the area above the person's ear to the rear of the person's head and holds at least one third electromagnetic energy sensor 713 (substantially located at an electrode position selected from the group consisting of Iz, P9, P7, TP9, and TP7), wherein the first, second, and third arms/portions are connected, and wherein the rear of the second arm/portion is higher than the rear of the third arm/portion.

In an example, the left side of a wearable device for measuring electromagnetic brain activity can comprise: a first arm/portion which spans from an area (directly) above a person's ear to the person's forehead and holds at least one first electromagnetic energy sensor (substantially located at an electrode position selected from the group consisting of FP1, FPz, AF7, F7, FT7, and T7); a second arm/portion which spans from the area above the person's ear to the rear of the person's head; and a third arm/portion which spans from the area above the person's ear to the rear of the person's head, wherein the first, second, and third arms/portions are connected, and wherein the rear of the second arm/portion is higher than the rear of the third arm/portion.

In an example, the left side of a wearable device for measuring electromagnetic brain activity can comprise: a first arm/portion which spans from an area (directly) above a person's ear to the person's forehead; a second arm/portion which spans from the area above the person's ear to the rear of the person's head and holds at least one second electromagnetic energy sensor (substantially located at an electrode position selected from the group consisting of P5, P3, P1, Pz, CP5, T7); and a third arm/portion which spans from the area above the person's ear to the rear of the person's head, wherein the first, second, and third arms/portions are connected, and wherein the rear of the second arm/portion is higher than the rear of the third arm/portion.

In an example, the left side of a wearable device for measuring electromagnetic brain activity can comprise: a first arm/portion which spans from an area (directly) above a person's ear to the person's forehead; a second arm/portion which spans from the area above the person's ear to the rear of the person's head; and a third arm/portion which spans from the area above the person's ear to the rear of the person's head and holds at least one third electromagnetic energy sensor (substantially located at an electrode position selected from the group consisting of Iz, P9, P7, TP9, and TP7), wherein the first, second, and third arms/portions are connected, and wherein the rear of the second arm/portion is higher than the rear of the third arm/portion.

In an example, the intersection of the second arm/portion and the third arm/portion can form a rear-facing angle between 5 and 65 degrees. In an example, the front of the first arm/portion can be higher than the rear of the third arm/portion. In an example, the three arms/portions can intersect at a location (directly) above the person's ear and/or at a location within 2″ of the front-to-back midpoint of the side of the person's head. In an example, the first arm/portion can have an upward-facing concavity. In an example, the second arm/portion can have an upwardly-facing concavity. In an example, this device can comprise a bifurcating ring around a person's head, wherein the ring bifurcates as it spans the rear of the person's head. In an example, the first and third arms/portions can comprise a continuous band around a person's head. In an example, this device can comprise a headband.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 8 shows the left side of a wearable device for measuring electromagnetic brain activity comprising: a ring 801 which encircles a person's head and spans the front and rear of their head, holding at least one electromagnetic energy sensor 811 (substantially located at an electrode position selected from the group consisting of O1, Oz, PO7, P7, F5, F3, F1, Fz, TP7, FT7, FC5, and T7).

In an example, a wearable device for measuring electromagnetic brain activity can comprise: a ring which encircles a person's head and holds at least one electromagnetic energy sensor 811 (substantially located at an electrode position selected from the group consisting of O1, Oz, PO7, P7, F5, F3, F1, Fz, TP7, FT7, FC5, and T7).

In an example, the front of the ring can be higher than the rear of the ring. In an example, a virtual plane which best fits this ring can intersect a horizontal plane when the person is standing up, forming a forward-facing angle between 5 and 75 degrees. In an example, the ring can have an upward-facing concavity. In an example, the ring can rest on the tops of the person's ears. In an example, the front of the ring can be more vertical than the rear of the ring. In an example, this ring can comprise a headband. In an example, this ring can comprise the perimeter of a skullcap or baseball cap.

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 9 shows the left side of a wearable device for measuring electromagnetic brain activity comprising: a ring and/or band 901 which encircles a person's head; one or more electromagnetic energy sensors 911 which are substantially located at electrode positions selected from the group consisting of O1, Oz, FP1, FPz, PO7, AF7, P7, F7, TP7, FT7, and T7; and prongs or teeth 912 which protrude into and/or under the person's hair at locations on the person's head which are covered by hair. In an example, a wearable device for measuring electromagnetic brain activity can comprise: a ring and/or band which encircles a person's head; and one or more electromagnetic energy sensors which are substantially located at electrode positions selected from the group consisting of O1, Oz, FP1, FPz, PO7, AF7, P7, F7, TP7, FT7, and T7, wherein one or more of the electromagnetic energy sensors have prongs or teeth which protrude into and/or under a person's hair.

In an example, the front of the ring and/or band can be higher than the rear of the ring and/or band. In an example, a virtual plane which best fits this ring and/or band can intersect a horizontal plane when the person is standing up, forming a forward-facing angle between 5 and 75 degrees. In an example, the front portion of the ring and/or band can have an upward-facing concavity. In an example, the ring and/or band can rest on the tops of the person's ears. In an example, a ring and/or band can comprise a headband. In an example, a ring and/or band can comprise the perimeter of a skullcap or baseball cap.

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent.

In an example, a wearable device for measuring electromagnetic brain activity can comprise: a wearable ring and/or band which is configured to encircle a person's head at an anterior acute angle in the range of 0 to 45 degrees with respect to a horizontal plane when the person's head is upright, wherein the ring and/or band has prongs and/or teeth which protrude into and/or under the person's hair; and a plurality of electromagnetic energy sensors on the ring and/or band; wherein one or more of the electromagnetic energy sensors are located at placement sites of Modified Combinatorial Nomenclature (MCN) electrode placement system which are selected from the group consisting of F3, F4, F7, F8, Fz, T3, T4, T5, T6, P3, P4, Pz, O1, O2, C3, C4, and Cz; and wherein one or more of the electromagnetic energy sensors are made from an elastic polymer.

In an example, a wearable device for measuring electromagnetic brain activity can comprise: a wearable ring and/or band which is configured to encircle a person's head at an anterior acute angle in the range of 0 to 45 degrees with respect to a horizontal plane when the person's head is upright; and a plurality of electromagnetic energy sensors on the ring and/or band; wherein one or more of the electromagnetic energy sensors are located at placement sites of Modified Combinatorial Nomenclature (MCN) electrode placement system which are selected from the group consisting of F3, F4, F7, F8, Fz, T3, T4, T5, T6, P3, P4, Pz, O1, O2, C3, C4, and Cz; wherein one or more of the electromagnetic energy sensors are made from a silicone material which has been impregnated, doped, coated, or embedded with metal; and wherein one or more of the electromagnetic energy sensors have prongs and/or teeth which protrude into and/or under the person's hair.

In an example, a wearable device for measuring electromagnetic brain activity can comprise: a wearable ring and/or band which is configured to encircle a person's head at an anterior acute angle in the range of 0 to 45 degrees with respect to a horizontal plane when the person's head is upright; and a plurality of electromagnetic energy sensors on the ring and/or band; wherein one or more of the electromagnetic energy sensors are located at placement sites of Modified Combinatorial Nomenclature (MCN) electrode placement system which are selected from the group consisting of O1, Oz, FP1, FPz, PO7, AF7, P7, F7, TP7, FT7, and T7; wherein one or more of the electromagnetic energy sensors are made from a silicone material which has been impregnated, doped, coated, or embedded with carbon; and wherein one or more of the electromagnetic energy sensors have prongs and/or teeth which protrude into and/or under the person's hair.

In an example, a wearable device for measuring electromagnetic brain activity can be embodied in a soft ring and/or band (such as headband) which is worn around a person's head to measure electromagnetic brain activity. In an example, a wearable device for measuring electromagnetic brain activity comprising a wearable ring and/or band which encircles a person's head at an anterior acute angle in the range of 0 to 45 degrees with respect to a horizontal plane (when the person's head is upright) and a plurality of electromagnetic energy sensors on the ring and/or band.

In an example, this device can further comprise prongs and/or teeth which protrude into and/or under the person's hair. This can enable good electromagnetic communication with the person's head at locations on the person's head which are covered by hair. In an example, the prongs and/or teeth can be made from a silicone material (such as PDMS) which has been impregnated, doped, coated, or embedded with conductive material (such as a metal and/or carbon) in order to provide consistent but comfortable contact with the person's head. In an example, conductive metal can be silver or aluminum. In an example, carbon material can be in the form of nanotubes or graphene. In an example, electromagnetic energy sensors can be located at placement sites of Modified Combinatorial Nomenclature (MCN) electrode placement system which are selected from the group consisting of F3, F4, F7, F8, FPz, Fz, T3, T4, T5, T6, P3, P4, Pz, O1, O2, C3, C4, and Cz. Other relevant variations and components discussed in other portions of this concurrent disclosure or prior disclosures incorporated herein by reference can also be applied to this example.

FIG. 10 shows the left side of a wearable device for measuring electromagnetic brain activity comprising: a ring 1001 which encircles a person's head at a level which is at least 1″ above the person's ears, wherein this ring holds at least one electromagnetic energy sensor 1011 (substantially located at an electrode position selected from the group consisting of PO3, AF3, AFz, P5, F5, CP5, FC5, and C5).

In an example, a wearable device for measuring electromagnetic brain activity can comprise: a ring which encircles a person's head at a level which is at least 1″ above the person's ears, wherein this ring holds at least one electromagnetic energy sensor (substantially located at an electrode position selected from the group consisting of PO3, AF3, AFz, P5, F5, CP5, FC5, and C5).

In an example, the front of the ring can be higher than the rear of the ring. In an example, a virtual plane which best fits this ring can intersect a horizontal plane when the person is standing up, forming a forward-facing angle between 5 and 75 degrees. In an example, the front portion of the ring can have an upward-facing concavity. In an example, this ring can comprise a headband. In an example, this ring can comprise the perimeter of a skullcap or baseball cap.

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 11 shows the left side of a wearable device for measuring electromagnetic brain activity comprising: a first arm/portion 1101 which spans from an area (directly) above a person's ear to the top of their head and holds at least one first electromagnetic energy sensor 1111 (substantially located at an electrode position selected from the group consisting of T7, C5, C3, C1, and Cz); a second arm/portion 1102 which curves around the front of the person's ear and holds at least one second electromagnetic energy sensor 1112 (substantially located at an electrode position selected from the group consisting of T9, T7); and a third arm/portion 1103 which curves around the rear of the person's ear and holds at least one third electromagnetic energy sensor 1113 (substantially located at an electrode position selected from the group consisting of P9, TP9, and TP7).

In an example, the left side of a wearable device for measuring electromagnetic brain activity can comprise: a first arm/portion which spans from an area (directly) above a person's ear to the top of their head and holds at least one first electromagnetic energy sensor (substantially located at an electrode position selected from the group consisting of T7, C5, C3, C1, and Cz); a second arm/portion which curves around the front of the person's ear; and a third arm/portion which curves around the rear of the person's ear.

In an example, the left side of a wearable device for measuring electromagnetic brain activity can comprise: a first arm/portion which spans from an area (directly) above a person's ear to the top of their head; a second arm/portion which curves around the front of the person's ear and holds at least one second electromagnetic energy sensor (substantially located at an electrode position selected from the group consisting of T9, T7); and a third arm/portion which curves around the rear of the person's ear.

In an example, the left side of a wearable device for measuring electromagnetic brain activity can comprise: a first arm/portion which spans from an area (directly) above a person's ear to the top of their head; a second arm/portion which curves around the front of the person's ear; and a third arm/portion which curves around the rear of the person's ear and holds at least one third electromagnetic energy sensor (substantially located at an electrode position selected from the group consisting of P9, TP9, and TP7).

In an example, this device can comprise a pair of headphones. In an example, the first arm/portion can loop over the top of the person's head. In an example, the second and third arms/portions can collectively span between one half and three-quarters of the circumference around a person's ear. In an example, the first, second, and third arms/portions of this device can intersect and/or be connected to each other at an area (directly) above the person's ear. In an example, the first arm/portion can be substantially vertical. In an example, the first arm/portion can have a forward-facing concavity. In an example, the first arm/portion can have a rear-facing concavity. In an example, the third arm/portion can also hold an electrode which is attached to the person's ear lobe.

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 12 shows the left side of a wearable device for measuring electromagnetic brain activity comprising: a first arm/portion 1201 which spans from a first location within 2″ of a person's ear to the top of the person's head and holds at least one first electromagnetic energy sensor 1211 (substantially located at an electrode position selected from the group consisting of FT7, FC5, FC3, FC1, FCz, and T7); a second arm/portion 1202 which spans from the first location to the rear of the person's head and holds at least one second electromagnetic energy sensor 1212 (substantially located at an electrode position selected from the group consisting of O1, Oz, PO7, P7, TP7, T7), wherein the first arm/portion and the second arm/portion are connected to each other at the first location; and a third arm/portion 1203 which spans between the first arm/portion and the second arm/portion at locations which are different than the first location and which holds at least one third electromagnetic energy sensor 1213 (substantially located at electrode position C5).

In an example, the left side of a wearable device for measuring electromagnetic brain activity can comprise: a first arm/portion which spans from a first location within 2″ of a person's ear to the top of the person's head and holds at least one first electromagnetic energy sensor (substantially located at an electrode position selected from the group consisting of FT7, FC5, FC3, FC1, FCz, and T7); a second arm/portion which spans from the first location to the rear of the person's head, wherein the first arm/portion and the second arm/portion are connected to each other at the first location; and a third arm/portion which spans between the first arm/portion and the second arm/portion at locations which are different than the first location.

In an example, the left side of a wearable device for measuring electromagnetic brain activity can comprise: a first arm/portion which spans from a first location within 2″ of a person's ear to the top of the person's head; a second arm/portion which spans from the first location to the rear of the person's head and holds at least one second electromagnetic energy sensor (substantially located at an electrode position selected from the group consisting of O1, Oz, PO7, P7, TP7, T7), wherein the first arm/portion and the second arm/portion are connected to each other at the first location; and a third arm/portion which spans between the first arm/portion and the second arm/portion at locations which are different than the first location.

In an example, the left side of a wearable device for measuring electromagnetic brain activity can comprise: a first arm/portion which spans from a first location within 2″ of a person's ear to the top of the person's head; a second arm/portion which spans from the first location to the rear of the person's head, wherein the first arm/portion and the second arm/portion are connected to each other at the first location; and a third arm/portion which spans between the first arm/portion and the second arm/portion at locations which are different than the first location and which holds at least one third electromagnetic energy sensor (substantially located at electrode position C5).

In an example, the first arm/portion loops over the top of the person's head. In an example, the second arm/portion loops around the rear of the person's head. In an example, the third arm/portion spans between the left-side mid-point of the first arm/portion and the left-side mid-point of the second arm/portion. In an example, the third arm/portion spans between midsections of the first and second arm/portions. In an example, the third arm/portion connects midsections of the first and second arm/portions. In an example, the first arm/portion can have a forward-facing concavity. In an example, the second arm/portion can have a downward-facing concavity. In an example, the best fitting virtual plane for the first arm/portion and the best fitting virtual plane for the second arm/portion can be substantially perpendicular to each other. In an example, this device can rest on the tops of the person's ears. In an example, a left-side view of this device can have the shape of an arcuate capital letter “A” which has been rotated clockwise between 100 and 170 degrees.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

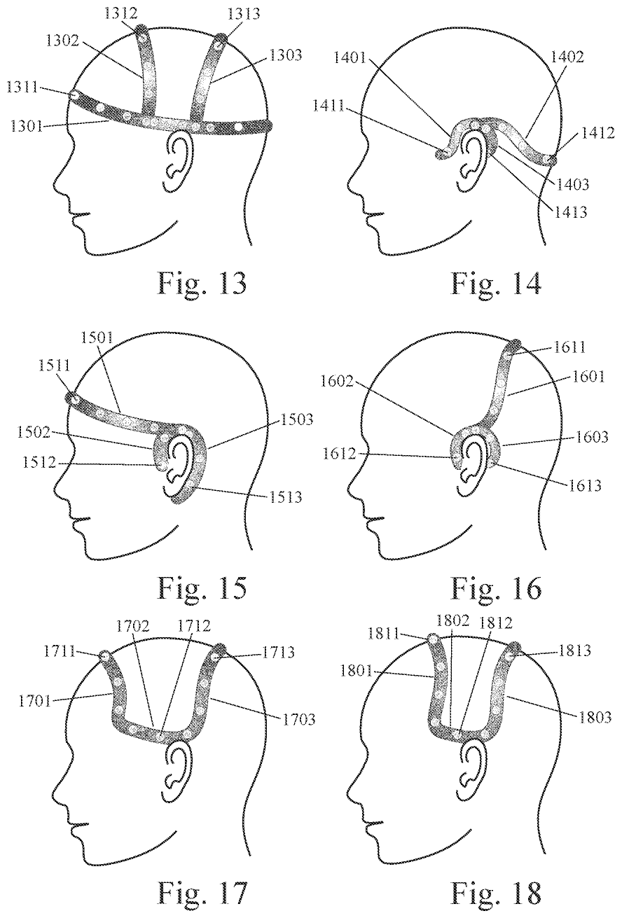

FIG. 13 shows the left side of a wearable device for measuring electromagnetic brain activity comprising: a ring 1301 which encircles a person's head and holds at least one first electromagnetic energy sensor 1311 (substantially located at an electrode position selected from the group consisting of O1, Oz, FP1, FPz, PO7, AF7, P7, F7, TP7, FT7, and T7); a front-upper arm/portion 1302 which loops upward from the ring over the top of the person's head and holds at least one second electromagnetic energy sensor 1312 (substantially located at an electrode position selected from the group consisting of FT7, FC5, FC3, FC1, and FCz); and a rear-upper arm/portion 1303 which loops upward from the ring over the top of the person's head and holds at least one third electromagnetic energy sensor 1313 (substantially located at an electrode position selected from the group consisting of TP7, CP5, CP3, CP1, and CPz), wherein the front-upper arm/portion is closer to the front of the person's head than the rear-upper arm/portion.

In an example, a wearable device for measuring electromagnetic brain activity can comprise: a ring which encircles a person's head and holds at least one first electromagnetic energy sensor (substantially located at an electrode position selected from the group consisting of O1, Oz, FP1, FPz, PO7, AF7, P7, F7, TP7, FT7, and T7); a front-upper arm/portion which loops upward from the ring over the top of the person's head; and a rear-upper arm/portion which loops upward from the ring over the top of the person's head, wherein the front-upper arm/portion is closer to the front of the person's head than the rear-upper arm/portion.

In an example, a wearable device for measuring electromagnetic brain activity can comprise: a ring which encircles a person's head; a front-upper arm/portion which loops upward from the ring over the top of the person's head and holds at least one second electromagnetic energy sensor (substantially located at an electrode position selected from the group consisting of FT7, FC5, FC3, FC1, and FCz); and a rear-upper arm/portion which loops upward from the ring over the top of the person's head, wherein the front-upper arm/portion is closer to the front of the person's head than the rear-upper arm/portion.

In an example, a wearable device for measuring electromagnetic brain activity can comprise: a ring which encircles a person's head; a front-upper arm/portion which loops upward from the ring over the top of the person's head; and a rear-upper arm/portion which loops upward from the ring over the top of the person's head and holds at least one third electromagnetic energy sensor (substantially located at an electrode position selected from the group consisting of TP7, CP5, CP3, CP1, and CPz), wherein the front-upper arm/portion is closer to the front of the person's head than the rear-upper arm/portion.

In an example, the ring can be tilted so that the front of the ring is higher than the rear of the ring. In an example, the ring can have an upward-facing concavity. In an example, the front-upper arm/portion can have a front-facing concavity. In an example, the rear-upper arm/portion can have a rear-facing concavity. In an example, the ring can rest on the tops of the person's ears. In an example, the front-upper arm/portion and the rear-upper arm/portion can diverge from each other as they leave the ring and loop over the person's head. In an example, the front-upper arm/portion can be anterior to a virtual vertical plane which connects a person's ears and the rear-upper arm/portion can be posterior relative to this virtual vertical plane.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection with the ring. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 14 shows the left side of a wearable device for measuring electromagnetic brain activity comprising: a first arm/portion 1401 which curves forward and downward from a person's ear and holds at least one first electromagnetic energy sensor 1411 (substantially located at an electrode position selected from the group consisting of T9 and T7); a second arm/portion 1402 which curves backward and downward from the person's ear and holds at least one second electromagnetic energy sensor 1412 (substantially located at an electrode position selected from the group consisting of Iz, P7, TP7); and a third arm/portion 1403 which loops around the rear of the person's head and holds at least one third electromagnetic energy sensor 1413 (substantially located at an electrode position selected from the group consisting of TP9 and TP7).

In an example, the left side of a wearable device for measuring electromagnetic brain activity can comprise: a first arm/portion which curves forward and downward from a person's ear and holds at least one first electromagnetic energy sensor (substantially located at an electrode position selected from the group consisting of T9 and T7); a second arm/portion which curves backward and downward from the person's ear; and a third arm/portion which loops around the rear of the person's head.

In an example, the left side of a wearable device for measuring electromagnetic brain activity can comprise: a first arm/portion which curves forward and downward from a person's ear; a second arm/portion which curves backward and downward from the person's ear and holds at least one second electromagnetic energy sensor (substantially located at an electrode position selected from the group consisting of Iz, P7, TP7); and a third arm/portion which loops around the rear of the person's head.

In an example, the left side of a wearable device for measuring electromagnetic brain activity can comprise: a first arm/portion which curves forward and downward from a person's ear; a second arm/portion which curves backward and downward from the person's ear; and a third arm/portion which loops around the rear of the person's head and holds at least one third electromagnetic energy sensor (substantially located at an electrode position selected from the group consisting of TP9 and TP7).

In an example, the first arm/portion curves forward and downward in a sinusoidal manner, comprising a partial or complete sinusoidal wave. In an example, the first arm/portion curves forward and downward from the top of the person's ear. In an example, the first arm/portion has a length between 2″ and 5″. In an example, the second arm/portion curves backward and downward from the top of the person's ear. In an example, the second arm/portion curves around the rear of the person's ear. In an example, the third arm/portion tilts downward as is spans from the person's ear to the rear of the person's head. In an example, the first, second, and third arms/portions are connected and/or joined at a location (directly) above the person's ear. In an example, the device can rest on the tops of the person's ears.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 15 shows the left side of a wearable device for measuring electromagnetic brain activity comprising: a forehead arm/portion 1501 which spans from a person's ear to their forehead and holds at least one first electromagnetic energy sensor 1511 (substantially located at an electrode position selected from the group consisting of FP1, FPz, AF7, F7, FT7, and T7); a front-ear arm/portion 1502 which curves around the front of the person's ear and holds at least one second electromagnetic energy sensor 1512 (substantially located at an electrode position selected from the group consisting of T9 and T7); and a rear-ear arm/portion 1503 which curves around the rear of the person's ear and holds at least one third electromagnetic energy sensor 1513 (substantially located at an electrode position selected from the group consisting of TP9, TP7, and T7).

In an example, the left side of a wearable device for measuring electromagnetic brain activity can comprise: a forehead arm/portion which spans from a person's ear to their forehead and holds at least one first electromagnetic energy sensor (substantially located at an electrode position selected from the group consisting of FP1, FPz, AF7, F7, FT7, and T7); a front-ear arm/portion which curves around the front of the person's ear; and a rear-ear arm/portion which curves around the rear of the person's ear.

In an example, the left side of a wearable device for measuring electromagnetic brain activity can comprise: a forehead arm/portion which spans from a person's ear to their forehead; a front-ear arm/portion which curves around the front of the person's ear and holds at least one second electromagnetic energy sensor (substantially located at an electrode position selected from the group consisting of T9 and T7); and a rear-ear arm/portion which curves around the rear of the person's ear.

In an example, the left side of a wearable device for measuring electromagnetic brain activity can comprise: a forehead arm/portion which spans from a person's ear to their forehead; a front-ear arm/portion which curves around the front of the person's ear; and a rear-ear arm/portion which curves around the rear of the person's ear and holds at least one third electromagnetic energy sensor (substantially located at an electrode position selected from the group consisting of TP9, TP7, and T7).

In an example, the forehead arm/portion can loop laterally (e.g. left to right) around the person's forehead. In an example, the three arms/portions of this device can connect and/or intersect at a location (directly) above the person's ear. In an example, front-ear and rear-ear arms/portions can combine to span between 50% and 75% of the circumference of the person's ear. In an example, front-ear and rear-ear can combine to span between 66% and 95% of the circumference of the person's ear. In an example, the forehead arm/portion can have an upward-facing concavity. In an example, the device can rest on the tops of the person's ears.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 16 shows the left side of a wearable device for measuring electromagnetic brain activity comprising: an upper loop 1601 which loops over a person' head from a location within 2″ of a person's ear and holds at least one first electromagnetic energy sensor 1611 (substantially located at an electrode position selected from the group consisting of TP7, CP5, CP3, CP1, and CPz); a front-ear arm/portion 1602 which curves around the front of the person's ear and holds at least one second electromagnetic energy sensor 1612 (substantially located at an electrode position selected from the group consisting of T9 and T7); and a rear-ear arm/portion 1603 which curves around the rear of the person's ear and holds at least one third electromagnetic energy sensor 1613 (substantially located at an electrode position selected from the group consisting of TP9 and TP7).

In an example, the left side of a wearable device for measuring electromagnetic brain activity can comprise: an upper loop which loops over a person' head from a location within 2″ of a person's ear and holds at least one first electromagnetic energy sensor (substantially located at an electrode position selected from the group consisting of TP7, CP5, CP3, CP1, and CPz); a front-ear arm/portion which curves around the front of the person's ear; and a rear-ear arm/portion which curves around the rear of the person's ear.

In an example, the left side of a wearable device for measuring electromagnetic brain activity can comprise: an upper loop which loops over a person' head from a location within 2″ of a person's ear; a front-ear arm/portion which curves around the front of the person's ear and holds at least one second electromagnetic energy sensor (substantially located at an electrode position selected from the group consisting of T9 and T7); and a rear-ear arm/portion which curves around the rear of the person's ear.

In an example, the left side of a wearable device for measuring electromagnetic brain activity can comprise: an upper loop which loops over a person' head from a location within 2″ of a person's ear; a front-ear arm/portion which curves around the front of the person's ear; and a rear-ear arm/portion which curves around the rear of the person's ear and holds at least one third electromagnetic energy sensor (substantially located at an electrode position selected from the group consisting of TP9 and TP7).

In an example, the upper loop can loop over a person's head from a location (directly) above a person's ear. In an example, the upper loop can have a forward-facing concavity. In an example, the upper loop can have a sinusoidal shape. In an example, the upper loop can span the top of the person's head at a location to the rear of a virtual vertical plane which connects the person's ears. In an example, front-ear and rear-ear arms/portions can combine to span between 50% and 75% of the circumference of the person's ear. In an example, front-ear and rear-ear arms/portions can combine to span between 66% and 95% of the circumference of the person's ear. In an example, the device can rest on the tops of the person's ears. In an example, the device can comprise (or just be shaped like) a set of headphones.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 17 shows the left side of a wearable device for measuring electromagnetic brain activity comprising: a forward arm/portion 1701 which loops around an upper-frontal area of a person's head and holds at least one first electromagnetic energy sensor 1711 (substantially located at an electrode position selected from the group consisting of F7, F5, F3, F1, and Fz); a middle arm/portion 1702 which laterally spans a portion of the side of the person's head above their ear and holds at least one second electromagnetic energy sensor 1712 (substantially located at an electrode position selected from the group consisting of F7, TP7, FT7, and T7); and a rear arm/portion 1703 which loops around an upper-rear area of the person's head and holds at least one third electromagnetic energy sensor 1713 (substantially located at an electrode position selected from the group consisting of TP7, CP5, CP3, CP1, and CPz), wherein the middle arm/portion connects the forward arm/portion and the rear arm/portion.

In an example, the left side of a wearable device for measuring electromagnetic brain activity can comprise: a forward arm/portion which loops around an upper-frontal area of a person's head and holds at least one first electromagnetic energy sensor (substantially located at an electrode position selected from the group consisting of F7, F5, F3, F1, and Fz); a middle arm/portion which laterally spans a portion of the side of the person's head above their ear; and a rear arm/portion which loops around an upper-rear area of the person's head, wherein the middle arm/portion connects the forward arm/portion and the rear arm/portion.

In an example, the left side of a wearable device for measuring electromagnetic brain activity can comprise: a forward arm/portion which loops around an upper-frontal area of a person's head; a middle arm/portion which laterally spans a portion of the side of the person's head above their ear and holds at least one second electromagnetic energy sensor (substantially located at an electrode position selected from the group consisting of F7, TP7, FT7, and T7); and a rear arm/portion which loops around an upper-rear area of the person's head, wherein the middle arm/portion connects the forward arm/portion and the rear arm/portion.

In an example, the left side of a wearable device for measuring electromagnetic brain activity can comprise: a forward arm/portion which loops around an upper-frontal area of a person's head; a middle arm/portion which laterally spans a portion of the side of the person's head above their ear; and a rear arm/portion which loops around an upper-rear area of the person's head and holds at least one third electromagnetic energy sensor (substantially located at an electrode position selected from the group consisting of TP7, CP5, CP3, CP1, and CPz), wherein the middle arm/portion connects the forward arm/portion and the rear arm/portion.

In an example, the centroid of the middle arm/portion is below the centroid of the forward arm/portion and the centroid of the rear arm/portion. In an example, the middle arm/portion can rest on top of the person's ear. In an example, the device can be shaped like a saddle on top of the person's head. In an example, the forward arm/portion can span the top of the person's head at a location which is anterior to a virtual vertical plane which connects the person's ears. In an example, the rear arm/portion can span the top of the person's head at a location which is posterior to a virtual vertical plane which connects the person's ears. In an example, the forward arm/portion can have a forward-facing concavity. In an example, the rear arm/portion can have a rear-facing concavity. In an example, the middle arm/portion, the forward arm/portion, and the rear arm/portion can each have substantially the same length.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 18 shows the left side of a wearable device for measuring electromagnetic brain activity comprising: a forward arm/portion 1801 which loops around an upper-frontal area of a person's head and holds at least one first electromagnetic energy sensor 1811 (substantially located at an electrode position selected from the group consisting of FT7, FC5, FC3, FC1, and FCz); a middle arm/portion 1802 which laterally spans a portion of the side of the person's head above their ear and holds at least one second electromagnetic energy sensor 1812 (substantially located at an electrode position selected from the group consisting of TP7, FT7, and T7); and a rear arm/portion 1803 which loops around an upper-rear area of the person's head and holds at least one third electromagnetic energy sensor 1813 (substantially located at an electrode position selected from the group consisting of TP7, CP5, CP3, CP1, and CPz), wherein the middle arm/portion connects the forward arm/portion and the rear arm/portion.

In an example, the left side of a wearable device for measuring electromagnetic brain activity can comprise: a forward arm/portion which loops around an upper-frontal area of a person's head and holds at least one first electromagnetic energy sensor (substantially located at an electrode position selected from the group consisting of FT7, FC5, FC3, FC1, and FCz); a middle arm/portion which laterally spans a portion of the side of the person's head above their ear; and a rear arm/portion which loops around an upper-rear area of the person's head, wherein the middle arm/portion connects the forward arm/portion and the rear arm/portion.

In an example, the left side of a wearable device for measuring electromagnetic brain activity can comprise: a forward arm/portion which loops around an upper-frontal area of a person's head; a middle arm/portion which laterally spans a portion of the side of the person's head above their ear and holds at least one second electromagnetic energy sensor (substantially located at an electrode position selected from the group consisting of TP7, FT7, and T7); and a rear arm/portion which loops around an upper-rear area of the person's head, wherein the middle arm/portion connects the forward arm/portion and the rear arm/portion.

In an example, the left side of a wearable device for measuring electromagnetic brain activity can comprise: a forward arm/portion which loops around an upper-frontal area of a person's head; a middle arm/portion which laterally spans a portion of the side of the person's head above their ear; and a rear arm/portion which loops around an upper-rear area of the person's head and holds at least one third electromagnetic energy sensor (substantially located at an electrode position selected from the group consisting of TP7, CP5, CP3, CP1, and CPz), wherein the middle arm/portion connects the forward arm/portion and the rear arm/portion.

In an example, the centroid of the middle arm/portion is below the centroid of the forward arm/portion and the centroid of the rear arm/portion. In an example, the middle arm/portion can rest on top of the person's ear. In an example, the device can be shaped like a saddle on top of the person's head. In an example, the forward arm/portion can span the top of the person's head at a location which is anterior to a virtual vertical plane which connects the person's ears. In an example, the rear arm/portion can span the top of the person's head at a location which is posterior to a virtual vertical plane which connects the person's ears. In an example, the forward arm/portion can have a forward-facing concavity. In an example, the rear arm/portion can have a rear-facing concavity. In an example, the middle arm/portion can be shorter than either the forward arm/portion or the rear arm/portion.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

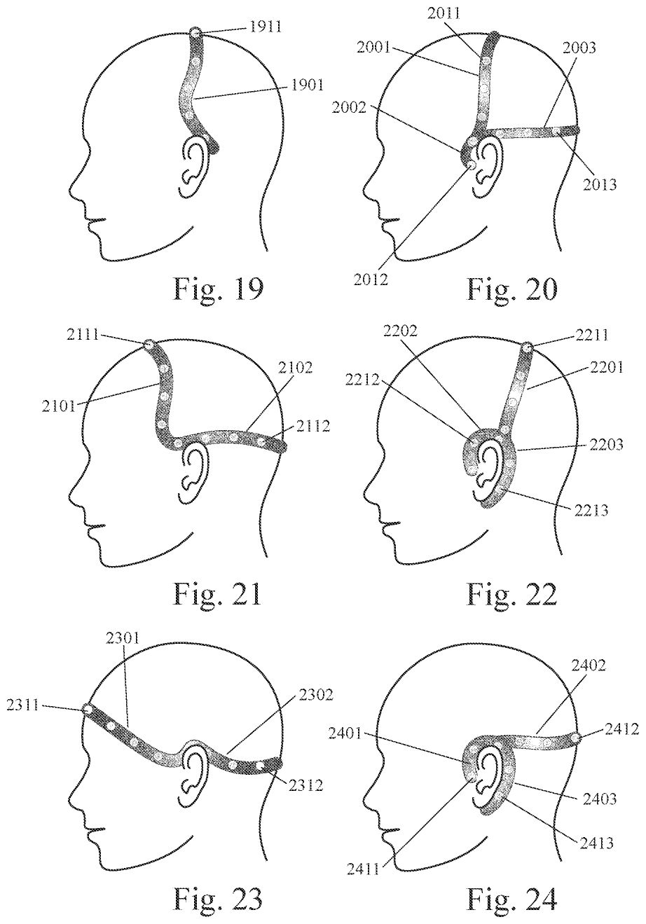

FIG. 19 shows the left side of a wearable device for measuring electromagnetic brain activity comprising: an upper loop 1901 which loops over the top of a person's head from one side (e.g. left) side to the other side (e.g. right) and wherein holds at least one first electromagnetic energy sensor 1911 (substantially located at an electrode position selected from the group consisting of TP9, TP7, T7, C5, C3, C1, and Cz).

In an example, the upper loop can loop over the top of the person's head from one ear to the other ear. In an example, the upper loop can loop over the top of the person's head from a location (directly) above an ear. In an example, this loop can have a rear-facing concavity. In an example, this loop can have a forward-facing concavity. In an example, this loop can have a sinusoidal shape. In an example, the virtual plane which best fits the upper loop can be substantially vertical. In an example, the virtual plane which best fits the upper loop can be a virtual vertical plane which connects the person's ears. In an example, the upper loop can cross the top of the person's head in a virtual vertical plane which connects the person's ears. In an example, the virtual plane which best fits the upper loop can intersect a vertical plane at an acute angle (e.g. between 5 and 45 degrees).

In an example, the right side of this mobile device (not shown) can be laterally symmetric with respect to the left side. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 20 shows the left side of a wearable device for measuring electromagnetic brain activity comprising: an upper loop 2001 which loops over the top of a person's head (from a location over the person's ear) and holds at least one first electromagnetic energy sensor 2011 (substantially located at an electrode position selected from the group consisting of T7, C5, C3, C1, and Cz); a rear loop 2002 which loops around the rear of the person's head (from the location over the person's ear) and holds at least one second electromagnetic energy sensor 2012 (substantially located at an electrode position selected from the group consisting of T9 and T7); an front-ear arm/portion 2003 which curves around the front of the person's ear and holds at least one third electromagnetic energy sensor 2013 (substantially located at an electrode position selected from the group consisting of O1, Oz, PO7, P7, TP7, and T7).

In an example, the left side of a wearable device for measuring electromagnetic brain activity can comprise: an upper loop which loops over the top of a person's head (from a location over the person's ear) and holds at least one first electromagnetic energy sensor (substantially located at an electrode position selected from the group consisting of T7, C5, C3, C1, and Cz); a rear loop which loops around the rear of the person's head (from the location over the person's ear); an front-ear arm/portion which curves around the front of the person's ear.

In an example, the left side of a wearable device for measuring electromagnetic brain activity can comprise: an upper loop which loops over the top of a person's head (from a location over the person's ear); a rear loop which loops around the rear of the person's head (from the location over the person's ear) and holds at least one second electromagnetic energy sensor (substantially located at an electrode position selected from the group consisting of T9 and T7); an front-ear arm/portion which curves around the front of the person's ear.

In an example, the left side of a wearable device for measuring electromagnetic brain activity can comprise: an upper loop which loops over the top of a person's head (from a location over the person's ear); a rear loop which loops around the rear of the person's head (from the location over the person's ear); an front-ear arm/portion which curves around the front of the person's ear and holds at least one third electromagnetic energy sensor (substantially located at an electrode position selected from the group consisting of O1, Oz, PO7, P7, TP7, and T7).

In an example, the upper loop can loop over the top of the person's head from one ear to the other ear. In an example, the upper loop can loop over the top of the person's head from a location (directly) above an ear. In an example, this loop can have a rear-facing concavity. In an example, this loop can have a forward-facing concavity. In an example, this loop can have a sinusoidal shape. In an example, the virtual plane which best fits the upper loop can be substantially vertical. In an example, the virtual plane which best fits the upper loop can be a virtual vertical plane which connects the person's ears. In an example, the upper loop can cross the top of the person's head in a virtual vertical plane which connects the person's ears. In an example, the virtual plane which best fits the upper loop can intersect a vertical plane at an acute angle (e.g. between 5 and 45 degrees). In an example, the upper loop, rear loop, and front-ear arm/portion can intersect and/or connect at a location within 2″ of the person's ear.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 21 shows the left side of a wearable device for measuring electromagnetic brain activity comprising: a first arm/portion 2101 which spans from a location within 2″ of a person's ear to the top of the person's head and holds at least one first electromagnetic energy sensor 2111 (substantially located at an electrode position selected from the group consisting of FT7, FC5, FC3, FC1, FCz, and T7); and a second arm/portion 2102 which spans from the location to the rear of the person's head and holds at least one second electromagnetic energy sensor 2112 (substantially located at an electrode position selected from the group consisting of Iz, O1, Oz, PO7, P7, TP7, and T7).

In an example, the left side of a wearable device for measuring electromagnetic brain activity can comprise: a first arm/portion which spans from a location within 2″ of a person's ear to the top of the person's head and holds at least one first electromagnetic energy sensor (substantially located at an electrode position selected from the group consisting of FT7, FC5, FC3, FC1, FCz, and T7); and a second arm/portion which spans from the location to the rear of the person's head.

In an example, the left side of a wearable device for measuring electromagnetic brain activity can comprise: a first arm/portion which spans from a location within 2″ of a person's ear to the top of the person's head; and a second arm/portion which spans from the location to the rear of the person's head and holds at least one second electromagnetic energy sensor (substantially located at an electrode position selected from the group consisting of Iz, O1, Oz, PO7, P7, TP7, and T7).

In an example, the first arm/portion can loop over the top of the person's head. In an example, the first arm/portion can have a forward-facing concavity. In an example, the second arm/portion can loop around the rear of the person's head. In an example, the second arm/portion can have a downward-facing concavity. In an example, the device overall (including all portions) can have an upward-and-rear-facing concavity. In an example, the best fitting virtual plane for the first arm/portion and the best fitting virtual plane for the second arm/portion can be substantially perpendicular to each other. In an example, this device can rest on the tops of the person's ears. In an example, an overall device (including both left and right sides) can comprise an undulating (e.g. sinusoidal) ring around an upper rear portion of a person's head.

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 22 shows the left side of a wearable device for measuring electromagnetic brain activity comprising: a first arm/portion 2201 which spans from an area above a person's ear to the top of their head and holds at least one first electromagnetic energy sensor 2211 (substantially located at an electrode position selected from the group consisting of TP7, CP5, CP3, CP1, and CPz); a second arm/portion 2202 which curves around the front of the person's ear and holds at least one second electromagnetic energy sensor 2212 (substantially located at an electrode position selected from the group consisting of TP7, T9, and T7); and a third arm/portion 2203 which curves around the rear of the person's ear and holds at least one third electromagnetic energy sensor 2213 (substantially located at an electrode position selected from the group consisting of P9, TP9, and TP7).

In an example, the left side of a wearable device for measuring electromagnetic brain activity can comprise: a first arm/portion which spans from an area above a person's ear to the top of their head and holds at least one first electromagnetic energy sensor (substantially located at an electrode position selected from the group consisting of TP7, CP5, CP3, CP1, and CPz); a second arm/portion which curves around the front of the person's ear; and a third arm/portion which curves around the rear of the person's ear.

In an example, the left side of a wearable device for measuring electromagnetic brain activity can comprise: a first arm/portion which spans from an area above a person's ear to the top of their head; a second arm/portion which curves around the front of the person's ear and holds at least one second electromagnetic energy sensor (substantially located at an electrode position selected from the group consisting of TP7, T9, and T7); and a third arm/portion which curves around the rear of the person's ear.

In an example, the left side of a wearable device for measuring electromagnetic brain activity can comprise: a first arm/portion which spans from an area above a person's ear to the top of their head; a second arm/portion which curves around the front of the person's ear; and a third arm/portion which curves around the rear of the person's ear and holds at least one third electromagnetic energy sensor (substantially located at an electrode position selected from the group consisting of P9, TP9, and TP7).

In an example, this device can comprise a pair of headphones. In an example, the first arm/portion can loop over the top of the person's head. In an example, the first arm/portion can have a rearward tilt as it ascends the side of the person's head. In an alternative example, the first arm/portion can be substantially vertical. In an example, the first arm/portion can have a forward-facing concavity. In an example, the first arm/portion can have a rear-facing concavity. In an example, the second and third arms/portions can collectively span between one half and three-quarters of the circumference around a person's ear. In an example, the first, second, and third arms/portions of this device can intersect and/or be connected to each other at an area (directly) above the person's ear. In an example, the third arm/portion can also hold an electrode which is attached to the person's ear lobe.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 23 shows the left side of a wearable device for measuring electromagnetic brain activity comprising: a front portion/loop 2301, which spans from a person's ear to their forehead and holds at least one first electromagnetic energy sensor 2311 (substantially located at an electrode position selected from the group consisting of FP1, FPz, AF7, F9, F7, FT9, T9, and T7); and a rear portion/loop 2302 which spans from the person's ear to the rear of the person's head and holds at least one third electromagnetic energy sensor 2312 (substantially located at an electrode position selected from the group consisting of Iz, P9, TP9, and T7).

In an example, the left side of a wearable device for measuring electromagnetic brain activity can comprise: a front portion/loop which spans from a person's ear to their forehead and holds at least one first electromagnetic energy sensor (substantially located at an electrode position selected from the group consisting of FP1, FPz, AF7, F9, F7, FT9, T9, and T7); and a rear portion/loop which spans from the person's ear to the rear of the person's head.

In an example, the left side of a wearable device for measuring electromagnetic brain activity can comprise: a front portion/loop which spans from a person's ear to their forehead; and a rear portion/loop which spans from the person's ear to the rear of the person's head and holds at least one third electromagnetic energy sensor (substantially located at an electrode position selected from the group consisting of Iz, P9, TP9, and T7).

In an example, this device can comprise an undulating ring which encircles a person's head, spanning their forehead and the rear of their head, with upward undulations over the tops of the person's ears. In an example, this device can comprise an undulating ring which encircles a person's head, spanning their forehead and the rear of their head, with sinusoidal waves over the tops of the person's ears. In an example, this device can comprise a ring or headband around a person's head with downward-facing concave portions over the person's ears. In an example, the front portion/loop can loop around the person's forehead and the rear portion/loop can loop around the rear of the person's head. In an example, a virtual plane which best fits this undulating ring can intersect a horizontal plane when the person is standing up, forming a forward-facing angle between 5 and 75 degrees. In an example, the front of this undulating ring can be higher than the rear of this undulating ring. In an example, the rear of the undulating ring can be lower than the top of the person's ear. In an example, this device can comprise a headband or a cap.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 24 shows the left side of a wearable device for measuring electromagnetic brain activity comprising: a first arm/portion 2401 which curves around the front of a person's ear and holds at least one first electromagnetic energy sensor 2411 (substantially located at an electrode position selected from the group consisting of T9 and T7); a second arm/portion 2402 which curves around the rear of the person's ear and holds at least one second electromagnetic energy sensor 2412 (substantially located at an electrode position selected from the group consisting of O1, Oz, PO7, P7, TP7, and T7); a third arm/portion 2403 which extends from the person's ear to the rear of their head and holds at least one third electromagnetic energy sensor 2413 (substantially located at an electrode position selected from the group consisting of P9, TP9, and TP7).

In an example, the left side of a wearable device for measuring electromagnetic brain activity can comprise: a first arm/portion which curves around the front of a person's ear and holds at least one first electromagnetic energy sensor (substantially located at an electrode position selected from the group consisting of T9 and T7); a second arm/portion which curves around the rear of the person's ear; a third arm/portion which extends from the person's ear to the rear of their head.

In an example, the left side of a wearable device for measuring electromagnetic brain activity can comprise: a first arm/portion which curves around the front of a person's ear; a second arm/portion which curves around the rear of the person's ear and holds at least one second electromagnetic energy sensor (substantially located at an electrode position selected from the group consisting of O1, Oz, PO7, P7, TP7, and T7); a third arm/portion which extends from the person's ear to the rear of their head.

In an example, the left side of a wearable device for measuring electromagnetic brain activity can comprise: a first arm/portion which curves around the front of a person's ear; a second arm/portion which curves around the rear of the person's ear; a third arm/portion which extends from the person's ear to the rear of their head and holds at least one third electromagnetic energy sensor (substantially located at an electrode position selected from the group consisting of P9, TP9, and TP7).

In an example, the first arm/portion curves forward and downward in a sinusoidal manner around the front of the person's ear, comprising a partial or complete sinusoidal wave. In an example, the first arm/portion has a length between 1″ and 3.″ In an example, the second arm/portion curves backward and downward from the top of the person's ear around the rear of the person's ear. In an example, the third arm/portion is relatively horizontal as loops around the rear of the person's head from ear to ear. In an example, the first, second, and third arms/portions are connected and/or joined at a location (directly) above the person's ear. In an example, this device can rest on the person's ears.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

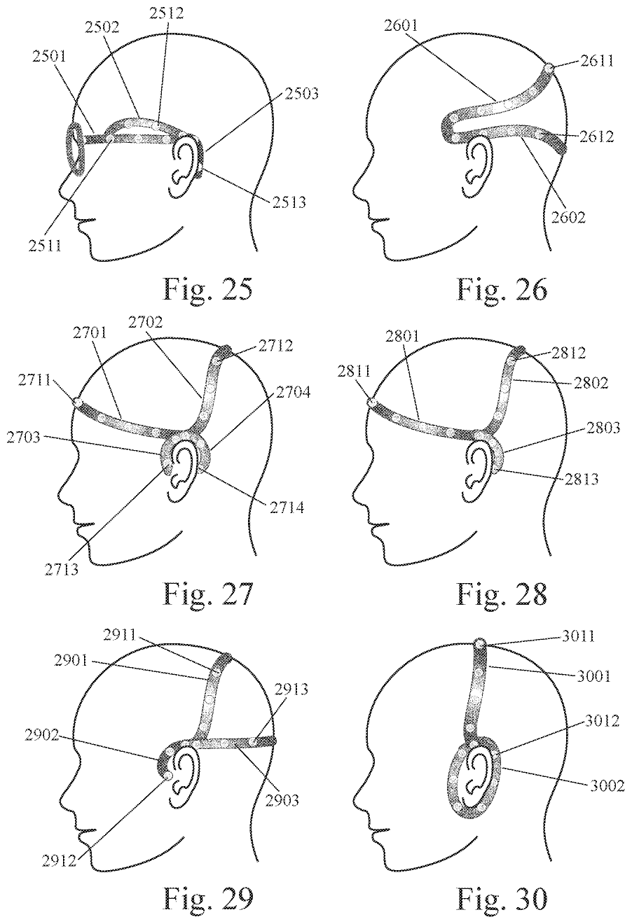

FIG. 25 shows the left side of a wearable device for measuring electromagnetic brain activity comprising: a lower branch of an eyewear sidepiece 2501 which holds at least one first electromagnetic energy sensor 2511 (substantially located at an electrode position selected from the group consisting of Nz, F9, FT9, FT7, and T7); an upper branch of the eyewear sidepiece 2502 which holds at least one second electromagnetic energy sensor 2512 (substantially located at an electrode position selected from the group consisting of AF7, F7, FT7, and T7); and a rear-ear arm of the eyewear sidepiece 2503 which holds at least one third electromagnetic energy sensor 2513 (substantially located at an electrode position selected from the group consisting of P9, TP9, and TP7).

In an example, the left side of a wearable device for measuring electromagnetic brain activity can comprise: a lower branch of an eyewear sidepiece which holds at least one first electromagnetic energy sensor (substantially located at an electrode position selected from the group consisting of Nz, F9, FT9, FT7, and T7); an upper branch of the eyewear sidepiece; and a rear-ear arm of the eyewear sidepiece.

In an example, the left side of a wearable device for measuring electromagnetic brain activity can comprise: a lower branch of an eyewear sidepiece; an upper branch of the eyewear sidepiece which holds at least one second electromagnetic energy sensor (substantially located at an electrode position selected from the group consisting of AF7, F7, FT7, and T7); and a rear-ear arm of the eyewear sidepiece.

In an example, the left side of a wearable device for measuring electromagnetic brain activity can comprise: a lower branch of an eyewear sidepiece; an upper branch of the eyewear sidepiece; and a rear-ear arm of the eyewear sidepiece which holds at least one third electromagnetic energy sensor (substantially located at an electrode position selected from the group consisting of P9, TP9, and TP7).

In an example, the upper branch of the sidepiece has a front end and a rear end which are both connected to the lower branch of the sidepiece. In an example, the lower branch of the sidepiece is relatively straight and the upper branch of the sidepiece is arcuate. In an example, the upper branch of the sidepiece has a sinusoidal shape. In an example, the upper branch of the sidepiece has a conic section shape. In an example, the sidepiece can be called the “temple” of a pair of eyeglasses. In an example, the upper branch is shorter than the lower branch.

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 26 shows the left side of a wearable device for measuring electromagnetic brain activity comprising: an upper arm/portion 2601 which spans from a location between a person's temple and the person's ear to the back of their head and holds at least one first electromagnetic energy sensor 2611 (substantially located at an electrode position selected from the group consisting of P5, P3, P1, Pz, CP5, FT7, FC5, and C5); and a lower arm/portion 2602 which also spans from the location to the back of their head and holds at least one second electromagnetic energy sensor 2612 (substantially located at an electrode position selected from the group consisting of Iz, O1, PO7, P7, TP7, FT7, and T7), wherein the front of the upper arm/portion is connected to the front of the lower arm/portion, and wherein the rear of the upper arm/portion is higher than the rear of the lower arm/portion.

In an example, the left side of a wearable device for measuring electromagnetic brain activity can comprise: an upper arm/portion which spans from a location between a person's temple and the person's ear to the back of their head and holds at least one first electromagnetic energy sensor (substantially located at an electrode position selected from the group consisting of P5, P3, P1, Pz, CP5, FT7, FC5, and C5); and a lower arm/portion which also spans from the location to the back of their head, wherein the front of the upper arm/portion is connected to the front of the lower arm/portion, and wherein the rear of the upper arm/portion is higher than the rear of the lower arm/portion.

In an example, the left side of a wearable device for measuring electromagnetic brain activity can comprise: an upper arm/portion which spans from a location between a person's temple and the person's ear to the back of their head and holds at least one first electromagnetic energy sensor; and a lower arm/portion which also spans from the location to the back of their head and holds at least one second electromagnetic energy sensor (substantially located at an electrode position selected from the group consisting of Iz, O1, PO7, P7, TP7, FT7, and T7), wherein the front of the upper arm/portion is connected to the front of the lower arm/portion, and wherein the rear of the upper arm/portion is higher than the rear of the lower arm/portion.

In an example, the upper arm/portion can have an upward-facing concavity and the lower arm/portion can have a downward-facing concavity. In an example, the device can span between one-half and two-thirds of the front-to-back width of the side of the person's head. In an example, the lower arms/portions of the device can rest on the person's ears. In an example, the device overall can have a rear-facing concavity. In an example, the left-side-portion of the device can have the shape of a rear-opening “bobby pin.”

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 27 shows the left side of a wearable device for measuring electromagnetic brain activity comprising: a first arm/portion 2701 which extends from a person's ear to their forehead (e.g. loops around their forehead) and holds at least one first electromagnetic energy sensor 2711 (substantially located at an electrode position selected from the group consisting of FP1, FPz, AF7, F7, TP7, FT7, and T7); a second arm/portion 2702 which extends from the person's ear to the top of their head (e.g. loops around the top of their head) and holds at least one second electromagnetic energy sensor 2712 (substantially located at an electrode position selected from the group consisting of TP7, CP5, CP3, CP1, and CPz); a third arm/portion 2703 which curves around the front of the person's ear and holds at least one third electromagnetic energy sensor 2713 (substantially located at an electrode position selected from the group consisting of T9 and T7); and a fourth arm/portion 2704 which curves around the rear of the person's ear and holds at least one fourth electromagnetic energy sensor 2714 (substantially located at an electrode position selected from the group consisting of P9, TP9, and TP7).

In an example, the left side of a wearable device for measuring electromagnetic brain activity can comprise: a first arm/portion which extends from a person's ear to their forehead (e.g. loops around their forehead) and holds at least one first electromagnetic energy sensor (substantially located at an electrode position selected from the group consisting of FP1, FPz, AF7, F7, TP7, FT7, and T7); a second arm/portion which extends from the person's ear to the top of their head (e.g. loops around the top of their head); a third arm/portion which curves around the front of the person's ear; and a fourth arm/portion which curves around the rear of the person's ear.

In an example, the left side of a wearable device for measuring electromagnetic brain activity can comprise: a first arm/portion which extends from a person's ear to their forehead (e.g. loops around their forehead); a second arm/portion which extends from the person's ear to the top of their head (e.g. loops around the top of their head) and holds at least one second electromagnetic energy sensor (substantially located at an electrode position selected from the group consisting of TP7, CP5, CP3, CP1, and CPz); a third arm/portion which curves around the front of the person's ear; and a fourth arm/portion which curves around the rear of the person's ear.

In an example, the left side of a wearable device for measuring electromagnetic brain activity can comprise: a first arm/portion which extends from a person's ear to their forehead (e.g. loops around their forehead); a second arm/portion which extends from the person's ear to the top of their head (e.g. loops around the top of their head); a third arm/portion which curves around the front of the person's ear and holds at least one third electromagnetic energy sensor (substantially located at an electrode position selected from the group consisting of T9 and T7); and a fourth arm/portion which curves around the rear of the person's ear.

In an example, the left side of a wearable device for measuring electromagnetic brain activity can comprise: a first arm/portion which extends from a person's ear to their forehead (e.g. loops around their forehead); a second arm/portion which extends from the person's ear to the top of their head (e.g. loops around the top of their head); a third arm/portion which curves around the front of the person's ear; and a fourth arm/portion which curves around the rear of the person's ear and holds at least one fourth electromagnetic energy sensor (substantially located at an electrode position selected from the group consisting of P9, TP9, and TP7).

In an example, the first arm/portion can loop around the person's forehead. In an example, the second arm/portion can loop around the top of the person's head. In an example, the best fitting virtual plane for the first arm/portion and the best fitting virtual plane for the second arm/portion can be substantially perpendicular to each other. In an example, the first arm/portion can have an upward-facing concavity. In an example, the second arm/portion can have a rear-facing concavity. In an example, the second arm/portion can have a forward-facing concavity.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 28 shows the left side of a wearable device for measuring electromagnetic brain activity comprising: a first arm/portion 2801 which extends from a person's ear to their forehead (e.g. loops around their forehead) and holds at least one first electromagnetic energy sensor 2811 (substantially located at an electrode position selected from the group consisting of FP1, FPz, AF7, F7, TP7, FT7, and T7); a second arm/portion 2802 which extends from the person's ear to the top of their head (e.g. loops around the top of their head) and holds at least one second electromagnetic energy sensor 2812 (substantially located at an electrode position selected from the group consisting of TP7, CP5, CP3, CP1, CPz); and a third arm/portion 2803 which curves around the rear of the person's ear and holds at least one third electromagnetic energy sensor 2813 (substantially located at an electrode position selected from the group consisting of TP9, TP7, and T7).

In an example, the left side of a wearable device for measuring electromagnetic brain activity can comprise: a first arm/portion which extends from a person's ear to their forehead (e.g. loops around their forehead) and holds at least one first electromagnetic energy sensor (substantially located at an electrode position selected from the group consisting of FP1, FPz, AF7, F7, TP7, FT7, and T7); a second arm/portion which extends from the person's ear to the top of their head (e.g. loops around the top of their head); and a third arm/portion which curves around the rear of the person's ear.

In an example, the left side of a wearable device for measuring electromagnetic brain activity can comprise: a first arm/portion which extends from a person's ear to their forehead (e.g. loops around their forehead); a second arm/portion which extends from the person's ear to the top of their head (e.g. loops around the top of their head) and holds at least one second electromagnetic energy sensor (substantially located at an electrode position selected from the group consisting of TP7, CP5, CP3, CP1, CPz); and a third arm/portion which curves around the rear of the person's ear.

In an example, the left side of a wearable device for measuring electromagnetic brain activity can comprise: a first arm/portion which extends from a person's ear to their forehead (e.g. loops around their forehead); a second arm/portion which extends from the person's ear to the top of their head (e.g. loops around the top of their head); and a third arm/portion which curves around the rear of the person's ear and holds at least one third electromagnetic energy sensor (substantially located at an electrode position selected from the group consisting of TP9, TP7, and T7).

In an example, the first arm/portion can loop around the person's forehead. In an example, the second arm/portion can loop around the top of the person's head. In an example, the best fitting virtual plane for the first arm/portion and the best fitting virtual plane for the second arm/portion can be substantially perpendicular to each other. In an example, the first arm/portion can have an upward-facing concavity. In an example, the second arm/portion can have a rear-facing concavity. In an example, the second arm/portion can have a forward-facing concavity.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 29 shows the left side of a wearable device for measuring electromagnetic brain activity comprising: a first arm/portion 2901 which loops over the top of a person's head from their ear and holds at least one first electromagnetic energy sensor 2911 (substantially located at an electrode position selected from the group consisting of TP7, CP5, CP3, CP1, and CPz); a second arm/portion 2902 which loops around the back of the person's head from their ear and holds at least one second electromagnetic energy sensor 2912 (substantially located at an electrode position selected from the group consisting of T9, T7); and a third arm/portion 2903 which curves around the front of the person's ear and holds at least one third electromagnetic energy sensor 2913 (substantially located at an electrode position selected from the group consisting of Iz, O1, Oz, PO7, P7, TP7, and T7).

In an example, the left side of a wearable device for measuring electromagnetic brain activity can comprise: a first arm/portion which loops over the top of a person's head from their ear and holds at least one first electromagnetic energy sensor (substantially located at an electrode position selected from the group consisting of TP7, CP5, CP3, CP1, and CPz); a second arm/portion which loops around the back of the person's head from their ear; and a third arm/portion which curves around the front of the person's ear.

In an example, the left side of a wearable device for measuring electromagnetic brain activity can comprise: a first arm/portion which loops over the top of a person's head from their ear; a second arm/portion which loops around the back of the person's head from their ear and holds at least one second electromagnetic energy sensor (substantially located at an electrode position selected from the group consisting of T9, T7); and a third arm/portion which curves around the front of the person's ear.

In an example, the left side of a wearable device for measuring electromagnetic brain activity can comprise: a first arm/portion which loops over the top of a person's head from their ear; a second arm/portion which loops around the back of the person's head from their ear; and a third arm/portion which curves around the front of the person's ear and holds at least one third electromagnetic energy sensor (substantially located at an electrode position selected from the group consisting of Iz, O1, Oz, PO7, P7, TP7, and T7).

In an example, the first arm/portion can have a rear-facing concavity. In an example, the first arm/portion can have an undulating shape. In an example, the first arm/portion can have a sinusoidal shape. In an example, the second arm/portion can be substantially level/horizontal. In an example, the virtual plane which best fits the first arm/portion can intersect the virtual plane which best fits the second arm/portion at a rear-facing angle between 45 and 90 degrees. In an example, the first arm/portion can loop over the top of the person's head. In an example, the first arm/portion can have posterior tilt as it extends upward to loop over the top of the person's head.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 30 shows the left side of a wearable device for measuring electromagnetic brain activity comprising: an upper loop 3001 which loops over a person's head from one ear to the other and holds at least one first electromagnetic energy sensor 3011 (substantially located at an electrode position selected from the group consisting of T7, C5, C3, C1, and Cz); and an ear loop 3002 which encircles the person' ear and holds at least one second electromagnetic energy sensor 3012 (substantially located at an electrode position selected from the group consisting of TP9, TP7, T9, and T7).

In an example, a wearable device for measuring electromagnetic brain activity can comprise: an upper loop which loops over a person's head from one ear to the other and holds at least one first electromagnetic energy sensor (substantially located at an electrode position selected from the group consisting of T7, C5, C3, C1, and Cz); and an ear loop which encircles the person' ear.

In an example, a wearable device for measuring electromagnetic brain activity can comprise: an upper loop which loops over a person's head from one ear to the other; and an ear loop which encircles the person' ear and holds at least one second electromagnetic energy sensor (substantially located at an electrode position selected from the group consisting of TP9, TP7, T9, and T7).

In an example the upper loop can be substantially vertical. In an example, the upper loop can have a forward-facing concavity. In an example, the upper loop can have a rear-facing concavity. In an example, the device can comprise (or be shaped like) a pair of headphones. In an example, the ear loop can hold an electrode in contact with the person's earlobe.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

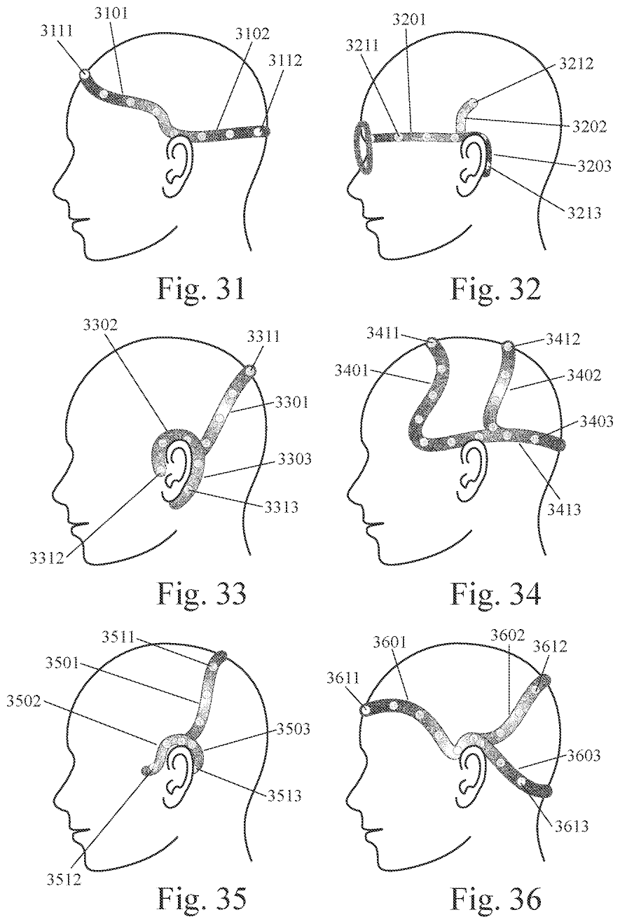

FIG. 31 shows the left side of a wearable device for measuring electromagnetic brain activity comprising: a forward loop/portion 3101 which loops around the front of a person's head from a location within 2″ of their ear and holds at least one first electromagnetic energy sensor 3111 (substantially located at an electrode position selected from the group consisting of AF3, AFz, F5, TP7, FC5, T7, and C5); and rear loop/portion 3102 which loops around the rear of the person's head from the location within 2″ of their ear and holds at least one second electromagnetic energy sensor 3112 (substantially located at an electrode position selected from the group consisting of O1, Oz, PO7, P7, TP7, and T7).

In an example, a wearable device for measuring electromagnetic brain activity can comprise: a forward loop/portion which loops around the front of a person's head from a location within 2″ of their ear and holds at least one first electromagnetic energy sensor (substantially located at an electrode position selected from the group consisting of AF3, AFz, F5, TP7, FC5, T7, and C5); and rear loop/portion which loops around the rear of the person's head from the location within 2″ of their ear.

In an example, a wearable device for measuring electromagnetic brain activity can comprise: a forward loop/portion which loops around the front of a person's head from a location within 2″ of their ear; and rear loop/portion which loops around the rear of the person's head from the location within 2″ of their ear and holds at least one second electromagnetic energy sensor (substantially located at an electrode position selected from the group consisting of O1, Oz, PO7, P7, TP7, and T7).

In an example, the forward loop/portion can have an undulating shape. In an example, the forward loop/portion can have a sinusoidal shape. In an example, the forward loop/portion can have a downward-facing concavity. In an example, the rear loop/portion can be substantially level/horizontal. In an example, the device can rest on a person's ears. In an example, the device can comprise an undulating ring which encircles a person's head. In an example, the device can comprise a partially-sinusoidal ring which encircles a person's head. In an example, the centroid of the forward loop/portion can be higher than the centroid of the rear loop/portion. In an example, the forward loop/portion can have an upward tilt as is extends forward from the person's ear to span the person's forehead.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 32 shows the left side of a wearable device for measuring electromagnetic brain activity comprising: a sidepiece of an eyewear frame 3201 which holds at least one first electromagnetic energy sensor 3211 (substantially located at an electrode position selected from the group consisting of Nz, F9, FT9, and T7); an upward arm/portion 3202 which extends upward from the sidepiece at a location within 2″ of the person's ear and holds at least one second electromagnetic energy sensor 3212 (substantially located at an electrode position selected from the group consisting of T7 and C5); and a rear-ear arm/portion 3203 which curves around the rear of the person's ear and holds at least one third electromagnetic energy sensor 3213 (substantially located at an electrode position selected from the group consisting of P9, TP9, TP7, and T7).

In an example, the left side of a wearable device for measuring electromagnetic brain activity can comprise: a sidepiece of an eyewear frame which holds at least one first electromagnetic energy sensor (substantially located at an electrode position selected from the group consisting of Nz, F9, FT9, and T7); an upward arm/portion which extends upward from the sidepiece at a location within 2″ of the person's ear; and a rear-ear arm/portion which curves around the rear of the person's ear.

In an example, the left side of a wearable device for measuring electromagnetic brain activity can comprise: a sidepiece of an eyewear frame; an upward arm/portion which extends upward from the sidepiece at a location within 2″ of the person's ear and holds at least one second electromagnetic energy sensor (substantially located at an electrode position selected from the group consisting of T7 and C5); and a rear-ear arm/portion which curves around the rear of the person's ear.

In an example, the left side of a wearable device for measuring electromagnetic brain activity can comprise: a sidepiece of an eyewear frame; an upward arm/portion which extends upward from the sidepiece at a location within 2″ of the person's ear; and a rear-ear arm/portion which curves around the rear of the person's ear and holds at least one third electromagnetic energy sensor (substantially located at an electrode position selected from the group consisting of P9, TP9, TP7, and T7).

In an example, the upward arm/portion can be arcuate. In an example, the upward arm/portion can have a rear-facing concavity. In an example, the upward arm/portion can extend upward to a location above the person's ear. In an example, the upward arm/portion can be have a first configuration in which it is aligned with the sidepiece and a second configuration in which it extends upward from the sidepiece. In an example, the upward arm/portion can be pivoted or rotated from its first configuration to its second configuration, or vice versa.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 33 shows the left side of a wearable device for measuring electromagnetic brain activity comprising: an upper-rear loop 3301 which loops, from their ears, around an upper-rear portion of a person's head and holds at least one first electromagnetic energy sensor 3311 (substantially located at an electrode position selected from the group consisting of P7, P5, P3, P1, and Pz); a front ear arm/portion 3302 which curves around the front of the person's ear and holds at least one second electromagnetic energy sensor 3312 (substantially located at an electrode position selected from the group consisting of TP7, T9, T7); and a rear ear arm/portion 3303 which holds at least one third electromagnetic energy sensor 3313 (substantially located at an electrode position selected from the group consisting of P9, TP9, and TP7).

In an example, a wearable device for measuring electromagnetic brain activity can comprise: an upper-rear loop which loops, from their ears, around an upper-rear portion of a person's head and holds at least one first electromagnetic energy sensor (substantially located at an electrode position selected from the group consisting of P7, P5, P3, P1, and Pz); a front ear arm/portion which curves around the front of the person's ear; and a rear ear arm/portion which curves around the rear of the person's ear.

In an example, a wearable device for measuring electromagnetic brain activity can comprise: an upper-rear loop which loops, from their ears, around an upper-rear portion of a person's head; a front ear arm/portion which curves around the front of the person's ear and holds at least one second electromagnetic energy sensor (substantially located at an electrode position selected from the group consisting of TP7, T9, T7); and a rear ear arm/portion which curves around the rear of the person's ear.

In an example, a wearable device for measuring electromagnetic brain activity can comprise: an upper-rear loop which loops, from their ears, around an upper-rear portion of a person's head; a front ear arm/portion which curves around the front of the person's ear; and a rear ear arm/portion which curves around the rear of the person's ear and holds at least one third electromagnetic energy sensor (substantially located at an electrode position selected from the group consisting of P9, TP9, and TP7).

In an example, the upper-rear loop can extend upward and rearward from a person's ear at an angle between 35 and 55 degrees relative a horizontal plane. In an example, the upper-rear loop can extend upward and rearward from a person's ear at an angle between 25 and 65 degrees relative a horizontal plane. In an example, the upper-rear loop can have a rear-facing concavity. In an example, the front ear arm/portion and the rear ear arm/portion can together span between 50% and 90% of the circumference of the person's ear.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 34 shows the left side of a wearable device for measuring electromagnetic brain activity comprising: an upper front loop 3401 which loops over the front half of the top of a person's head and holds at least one first electromagnetic energy sensor 3411 (substantially located at an electrode position selected from the group consisting of F9, F7, F5, FT9, FC3, FC1, FCz, T9, and T7); an upper rear loop 3402 which loops over the rear half of the top of a person's head and holds at least one second electromagnetic energy sensor 3412 (substantially located at an electrode position selected from the group consisting of P7, TP7, CP5, CP3, CP1, and CPz); and a mid-level arm/portion 3403 which connects the upper front loop to the upper rear loop, loops around the rear of the person's head, and holds at least one third electromagnetic energy sensor 3413 (substantially located at an electrode position selected from the group consisting of Iz, O1, Oz, PO7, P7, and TP7).

In an example, a wearable device for measuring electromagnetic brain activity can comprise: an upper front loop which loops over the front half of the top of a person's head and holds at least one first electromagnetic energy sensor (substantially located at an electrode position selected from the group consisting of F9, F7, F5, FT9, FC3, FC1, FCz, T9, and T7); an upper rear loop which loops over the rear half of the top of a person's head; and a mid-level arm/portion which connects the upper front loop to the upper rear loop and also loops around the rear of the person's head.

In an example, a wearable device for measuring electromagnetic brain activity can comprise: an upper front loop which loops over the front half of the top of a person's head; an upper rear loop which loops over the rear half of the top of a person's head and holds at least one second electromagnetic energy sensor (substantially located at an electrode position selected from the group consisting of P7, TP7, CP5, CP3, CP1, and CPz); and a mid-level arm/portion which connects the upper front loop to the upper rear loop and also loops around the rear of the person's head.

In an example, a wearable device for measuring electromagnetic brain activity can comprise: an upper front loop which loops over the front half of the top of a person's head; an upper rear loop which loops over the rear half of the top of a person's head; and a mid-level arm/portion which connects the upper front loop to the upper rear loop, loops around the rear of the person's head, and holds at least one third electromagnetic energy sensor (substantially located at an electrode position selected from the group consisting of Iz, O1, Oz, PO7, P7, and TP7).

In an example, the upper front loop can have a forward-facing concavity. In an example, the upper rear loop can have a forward-facing concavity. In an example, the upper front loop can be undulating (e.g. sinusoidal). In an example, the upper rear loop can be undulating (e.g. sinusoidal). In an example, the device can rest on the person's ears. In an example, the mid-level arm/portion can be substantially horizontal. In an example, the upper front loop can be forward of a vertical plane which connects the person's ears. In an example, the upper front loop can be to the rear of a vertical plane which connects the person's ears. In an example, a first virtual plane which best fits the upper front loop and a second virtual plane which best fits the upper rear loops can be substantially parallel.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 35 shows the left side of a wearable device for measuring electromagnetic brain activity comprising: an upper rear loop 3501 which loops, from a person's ears, around the rear half of the top of a person's head and holds at least one first electromagnetic energy sensor 3511 (substantially located at an electrode position selected from the group consisting of TP7, CP5, CP3, CP1, and CPz); a front ear arm/portion 3502 which extends forward from the person's ear and holds at least one second electromagnetic energy sensor 3512 (substantially located at an electrode position selected from the group consisting of TP7, T9, and T7); and a rear ear arm/portion 3503 which curves around the rear of the person's ear and holds at least one third electromagnetic energy sensor 3513 (substantially located at an electrode position selected from the group consisting of P9, TP9, and TP7).

In an example, a wearable device for measuring electromagnetic brain activity can comprise: an upper rear loop which loops, from a person's ears, around the rear half of the top of a person's head and holds at least one first electromagnetic energy sensor (substantially located at an electrode position selected from the group consisting of TP7, CP5, CP3, CP1, and CPz); a front ear arm/portion which extends forward from the person's ear; and a rear ear arm/portion which curves around the rear of the person's ear.

In an example, a wearable device for measuring electromagnetic brain activity can comprise: an upper rear loop which loops, from a person's ears, around the rear half of the top of a person's head; a front ear arm/portion which extends forward from the person's ear and holds at least one second electromagnetic energy sensor (substantially located at an electrode position selected from the group consisting of TP7, T9, and T7); and a rear ear arm/portion which curves around the rear of the person's ear.

In an example, a wearable device for measuring electromagnetic brain activity can comprise: an upper rear loop which loops, from a person's ears, around the rear half of the top of a person's head; a front ear arm/portion which extends forward from the person's ear; and a rear ear arm/portion which curves around the rear of the person's ear and holds at least one third electromagnetic energy sensor (substantially located at an electrode position selected from the group consisting of P9, TP9, and TP7).

In an example, the upper rear loop can extend upward and rearward from a person's ear at an angle between 25 and 55 degrees relative to a horizontal plane. In an example, the upper rear loop can extend upward and rearward from a person's ear at an angle between 45 and 75 degrees relative to a horizontal plane. In an example, an upper rear loop can be undulating (e.g. sinusoidal). In an example, an upper rear loop can have a forward-facing concavity. In an example, a front ear arm/portion can be undulating (e.g. sinusoidal). In an example, a front ear arm/portion can extend forward in a direct anterior manner/direction from the person's ear.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 36 shows the left side of a wearable device for measuring electromagnetic brain activity comprising: a front arm/portion 3601 which loops around a person's forehead from the person's ears and holds at least one first electromagnetic energy sensor 3611 (substantially located at an electrode position selected from the group consisting of FPz, AF3, F5, FT7, T9, and T7); an upper rear arm/portion 3602 from loops around an upper rear portion of the person's head from the person's ears and holds at least one second electromagnetic energy sensor 3612 (substantially located at an electrode position selected from the group consisting of P7, P5, P3, P1, Pz, TP7 and T7); and a lower rear arm/portion 3603 which loops around a lower rear portion of the person's head and holds at least one third electromagnetic energy sensor 3613 (substantially located at an electrode position selected from the group consisting of P9, TP9, and TP7).

In an example, a wearable device for measuring electromagnetic brain activity can comprise: a front arm/portion which loops around a person's forehead from the person's ears and holds at least one first electromagnetic energy sensor (substantially located at an electrode position selected from the group consisting of FPz, AF3, F5, FT7, T9, and T7); an upper rear arm/portion from loops around an upper rear portion of the person's head from the person's ears; and a lower rear arm/portion which loops around a lower rear portion of the person's head.

In an example, a wearable device for measuring electromagnetic brain activity can comprise: a front arm/portion which loops around a person's forehead from the person's ears; an upper rear arm/portion from loops around an upper rear portion of the person's head from the person's ears and holds at least one second electromagnetic energy sensor (substantially located at an electrode position selected from the group consisting of P7, P5, P3, P1, Pz, TP7 and T7); and a lower rear arm/portion which loops around a lower rear portion of the person's head.

In an example, a wearable device for measuring electromagnetic brain activity can comprise: a front arm/portion which loops around a person's forehead from the person's ears; an upper rear arm/portion from loops around an upper rear portion of the person's head from the person's ears; and a lower rear arm/portion which loops around a lower rear portion of the person's head and holds at least one third electromagnetic energy sensor (substantially located at an electrode position selected from the group consisting of P9, TP9, and TP7).

In an example, the front arm/portion and the lower rear arm/portion can together comprise an undulating (e.g. sinusoidal) ring which encircles the person's head. In an example, this ring can have a downward-facing concavity over the person's ear. In an example, the virtual plane which best fits this ring can intersect a horizontal plane (when the person is standing) at an angle between 5 and 45 degrees. In an example, the front arm/portion can have a downward-facing concavity. In an example, the lower rear arm/portion can have an upward-facing concavity. In an example, the upper rear arm/portion can have a forward-facing concavity.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

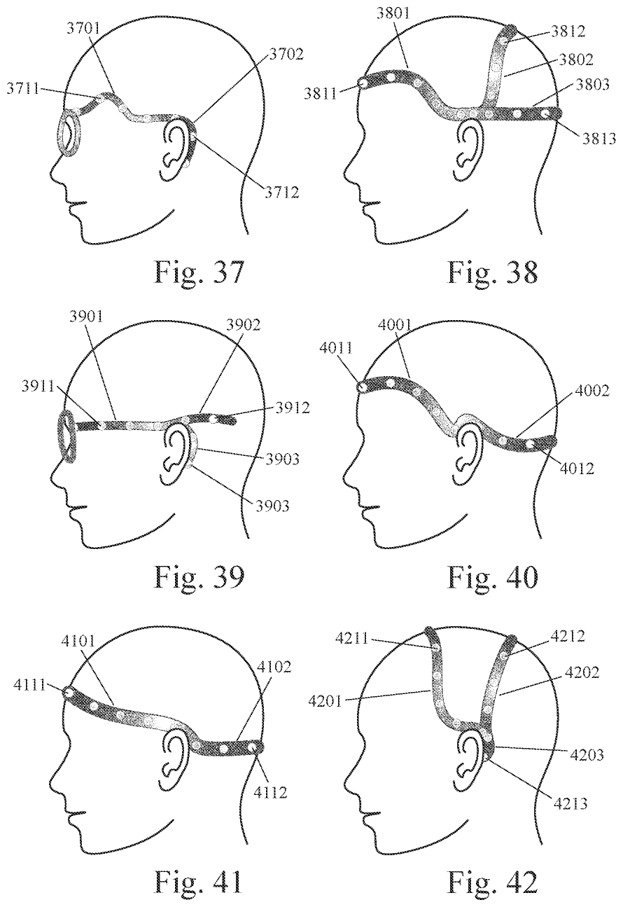

FIG. 37 shows the left side of an eyeglasses frame for sensing brain activity comprising: a sidepiece (e.g. sometimes called a “temple”) of the eyeglasses frame with an upward wave 3701 which holds at least one first electromagnetic energy sensor 3711 (substantially located at an electrode position selected from the group consisting of Nz, AF7, F9, F7, FT9, FT7, and T7) and a rear-ear portion 3702 which curves around the rear of the person's ear and holds at least one second electromagnetic energy sensor 3712 (substantially located at an electrode position selected from the group consisting of TP9, TP7, and T7).

In an example, the left side of an eyeglasses frame for sensing brain activity can comprise: a sidepiece of the eyeglasses frame with an upward wave which holds at least one first electromagnetic energy sensor (substantially located at an electrode position selected from the group consisting of Nz, AF7, F9, F7, FT9, FT7, and T7) and a rear-ear portion which curves around the rear of the person's ear.

In an example, the left side of an eyeglasses frame for sensing brain activity can comprise: a sidepiece of the eyeglasses frame with an upward wave and a rear-ear portion which curves around the rear of the person's ear and holds at least one second electromagnetic energy sensor (substantially located at an electrode position selected from the group consisting of TP9, TP7, and T7).

In an example, the upward wave of the sidepiece can be shaped like a conic section. In an example, the upward wave of the sidepiece can be a sinusoidal wave or a portion of a sinusoidal wave. In an example, the upward wave can extend onto (or over) a portion of the person's forehead and/or temple. In an example, the upward wave can extend inward (toward the person's head) as well as upward. In an example, the upward wave can hold an electromagnetic sensor in contact with the person's head. In an example, the upward wave can itself be electroconductive. In an example, the upward wave can be made from an electroconductive polymer. In an example, the upward wave can be made from an elastomeric polymer which is doped, impregnated, or coated with conductive material (such as silver or carbon particles).

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 38 shows the left side of a wearable device for measuring electromagnetic brain activity comprising: a front arm/portion 3801 which loops around a person's forehead from the person's ears and holds at least one first electromagnetic energy sensor 3811 (substantially located at an electrode position selected from the group consisting of FPz, AF3, F5, F3, TP7, FT7, FC5, and T7); an upper arm/portion 3802 which loops over the person's head and holds at least one second electromagnetic energy sensor 3812 (substantially located at an electrode position selected from the group consisting of TP7, CP5, CP3, CP1, CPz); and a rear arm/portion 3803 which loops around the rear of the person's and holds at least one third electromagnetic energy sensor 3813 (substantially located at an electrode position selected from the group consisting of O1, Oz, PO7, P7, and TP7).

In an example, a wearable device for measuring electromagnetic brain activity can comprise: a front arm/portion which loops around a person's forehead from the person's ears and holds at least one first electromagnetic energy sensor (substantially located at an electrode position selected from the group consisting of FPz, AF3, F5, F3, TP7, FT7, FC5, and T7); an upper arm/portion which loops over the person's head; and a rear arm/portion which loops around the rear of the person's head.

In an example, a wearable device for measuring electromagnetic brain activity can comprise: a front arm/portion which loops around a person's forehead from the person's ears; an upper arm/portion which loops over the person's head and holds at least one second electromagnetic energy sensor (substantially located at an electrode position selected from the group consisting of TP7, CP5, CP3, CP1, CPz); and a rear arm/portion which loops around the rear of the person's head.

In an example, a wearable device for measuring electromagnetic brain activity can comprise: a front arm/portion which loops around a person's forehead from the person's ears; an upper arm/portion which loops over the person's head; and a rear arm/portion which loops around the rear of the person's head and holds at least one third electromagnetic energy sensor (substantially located at an electrode position selected from the group consisting of O1, Oz, PO7, P7, and TP7).

In an example, the front arm/portion and the rear arm/portion can together comprise a ring which encircles the person's head. In an example, the front arm/portion and the rear arm/portion can together comprise an undulating ring which encircles the person's head. In an example, the front arm/portion can be higher than the rear arm/portion. In an example, the front arm/portion can have a downward-facing concavity. In an example, the rear arm/portion can be substantially level (e.g. horizontal when the person is standing up). In an example, the upper arm/portion can have a rear-facing concavity. In an example, the upper arm/portion can connect to the rear arm/portion at a location which is posterior to the person's ear.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 39 shows the left side of a wearable device for measuring electromagnetic brain activity comprising: eyeglasses with a sidepiece 3901 which holds at least one first electromagnetic energy sensor 3911 (substantially located at an electrode position selected from the group consisting of Nz, F9, FT9, and T7); a backward arm 3902, wherein the backward arm protrudes backward from the person's ear, but does not loop all the way around the rear of the person's head), and wherein the backward arm holds at least one second electromagnetic energy sensor 3912 (substantially located at an electrode position selected from the group consisting of PO7, P7, TP7, and T7); and a rear ear arm/portion 3903 which curves around the rear of the person's ear and holds at least one third electromagnetic energy sensor 3913 (substantially located at an electrode position selected from the group consisting of TP9, TP7, and T7).

In an example, the left side of a wearable device for measuring electromagnetic brain activity can comprise: eyeglasses with a sidepiece which holds at least one first electromagnetic energy sensor (substantially located at an electrode position selected from the group consisting of Nz, F9, FT9, and T7); a backward arm, wherein the backward arm protrudes backward from the person's ear, but does not loop all the way around the rear of the person's head; and a rear ear arm/portion which curves around the rear of the person's ear.

In an example, the left side of a wearable device for measuring electromagnetic brain activity can comprise: eyeglasses with a sidepiece; a backward arm, wherein the backward arm protrudes backward from the person's ear, but does not loop all the way around the rear of the person's head), and wherein the backward arm holds at least one second electromagnetic energy sensor (substantially located at an electrode position selected from the group consisting of PO7, P7, TP7, and T7); and a rear ear arm/portion which curves around the rear of the person's ear.

In an example, the left side of a wearable device for measuring electromagnetic brain activity can comprise: eyeglasses with a sidepiece; a backward arm, wherein the backward arm protrudes backward from the person's ear, but does not loop all the way around the rear of the person's head), and wherein the backward arm; and a rear ear arm/portion which curves around the rear of the person's ear and holds at least one third electromagnetic energy sensor (substantially located at an electrode position selected from the group consisting of TP9, TP7, and T7).

In an example, the backward arm can have a length in the range of 1″ to 4.″ In an example, the backward arm can extend between 33% and 66% of the way from the person's ear to the back of their head. In an example, the backward arm can be substantially co-linear with the sidepiece.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 40 shows the left side of a wearable device for measuring electromagnetic brain activity comprising: a forward loop 4001 which loops (from a person's ears) around the person's forehead and holds at least one first electromagnetic energy sensor 4011 (substantially located at an electrode position selected from the group consisting of FPz, AF3, F5, FT7, FC5, T9, and T7); and a rear loop 4002 which loops (from the person's ears) around the rear of a person's head and wherein this second arm/portion holds at least one second electromagnetic energy sensor 4012 (substantially located at an electrode position selected from the group consisting of Iz, P9, TP9, TP7, and T7).

In an example, a wearable device for measuring electromagnetic brain activity can comprise: a forward loop which loops (from a person's ears) around the person's forehead and holds at least one first electromagnetic energy sensor (substantially located at an electrode position selected from the group consisting of FPz, AF3, F5, FT7, FC5, T9, and T7); and a rear loop which loops (from the person's ears) around the rear of a person's head.

In an example, a wearable device for measuring electromagnetic brain activity can comprise: a forward loop which loops (from a person's ears) around the person's forehead; and a rear loop which loops (from the person's ears) around the rear of a person's head and wherein this second arm/portion holds at least one second electromagnetic energy sensor (substantially located at an electrode position selected from the group consisting of Iz, P9, TP9, TP7, and T7).

In an example, the forward loop and the rear loop can together comprise an undulating (e.g. sinusoidal) ring which encircles a person's head. In an example, the forward loop and the rear loop can together comprise an undulating ring which encircles a person's head with a downward-facing concavity over the person's ear. In an example, this ring can rest on a person's ears. In an example, the front loop can have a downward-facing concavity. In an example, the rear loop can have an upward-facing concavity. In an example, the front loop can be higher than the rear loop.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 41 shows the left side of a wearable device for measuring electromagnetic brain activity comprising: a front arm/portion 4101 which spans from a person's ear to their forehead and holds at least one first electromagnetic energy sensor 4111 (substantially located at an electrode position selected from the group consisting of FP1, FPz, AF7, F7, TP7, FT7, and T7); and a rear arm/portion 4102 which spans from the person's ear to the rear of their head and holds at least one second electromagnetic energy sensor 4112 (substantially located at an electrode position selected from the group consisting of Iz, P9, TP9, TP7, and T7).

In an example, the left side of a wearable device for measuring electromagnetic brain activity can comprise: a front arm/portion which spans from a person's ear to their forehead and holds at least one first electromagnetic energy sensor (substantially located at an electrode position selected from the group consisting of FP1, FPz, AF7, F7, TP7, FT7, and T7); and a rear arm/portion which spans from the person's ear to the rear of their head.

In an example, the left side of a wearable device for measuring electromagnetic brain activity can comprise: a front arm/portion which spans from a person's ear to their forehead; and a rear arm/portion which spans from the person's ear to the rear of their head and holds at least one second electromagnetic energy sensor (substantially located at an electrode position selected from the group consisting of Iz, P9, TP9, TP7, and T7).

In an example, the front arm/portion and the rear arm/portion can together comprise a ring which encircles the person's head. In an example, this ring can be an undulating ring. In an example, this ring can have sinusoidal waves. In an example, this ring can rest on top of the person's ears. In an example, the front arm/portion can be higher than the rear arm/portion. In an example, the centroid of the front arm/portion can be higher than the centroid of the rear arm/portion. In an example, the front arm/portion can extend forward and upward from the top of the person's ear to their forehead. In an example, the rear arm/portion can extend backward from the top or middle of the person's ear to the rear of their head. In an example, the rear arm/portion can extend backward and downward from the top or middle of the person's ear to the rear of their head.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 42 shows the left side of a wearable device for measuring electromagnetic brain activity comprising: an upper front arm/portion 4201 which loops (from a person's ears) over the top of the front half of the person's head and holds at least one first electromagnetic energy sensor 4211 (substantially located at an electrode position selected from the group consisting of FT7, FC5, FC3, FC1, FCz, and T7); an upper rear arm/portion 4202 which loops (from the person's ears) over the top of the rear half of the person's head and holds at least one second electromagnetic energy sensor 4212 (substantially located at an electrode position selected from the group consisting of TP9, TP7, CP5, CP3, CP1, and CPz); and a rear ear arm/portion 4203 which curves around the rear of the person's ear and holds at least one third electromagnetic energy sensor 4213 (substantially located at an electrode position selected from the group consisting of TP9, TP7, and T7).

In an example, a wearable device for measuring electromagnetic brain activity can comprise: an upper front arm/portion which loops (from a person's ears) over the top of the front half of the person's head and holds at least one first electromagnetic energy sensor (substantially located at an electrode position selected from the group consisting of FT7, FC5, FC3, FC1, FCz, and T7); an upper rear arm/portion which loops (from the person's ears) over the top of the rear half of the person's head; and a rear ear arm/portion which curves around the rear of the person's ear.

In an example, a wearable device for measuring electromagnetic brain activity can comprise: an upper front arm/portion which loops (from a person's ears) over the top of the front half of the person's head; an upper rear arm/portion which loops (from the person's ears) over the top of the rear half of the person's head and holds at least one second electromagnetic energy sensor (substantially located at an electrode position selected from the group consisting of TP9, TP7, CP5, CP3, CP1, and CPz); and a rear ear arm/portion which curves around the rear of the person's ear.

In an example, a wearable device for measuring electromagnetic brain activity can comprise: an upper front arm/portion which loops (from a person's ears) over the top of the front half of the person's head; an upper rear arm/portion which loops (from the person's ears) over the top of the rear half of the person's head; and a rear ear arm/portion which curves around the rear of the person's ear and holds at least one third electromagnetic energy sensor (substantially located at an electrode position selected from the group consisting of TP9, TP7, and T7).

In an example, this device can rest on (the tops of) the person's ears. In an example, the upper front arm/portion can have a rear-facing concavity. In an example, the upper rear arm/portion can have a rear-facing concavity. In an example, the upper front arm/portion and the upper rear arm/portion can diverge as they extend upward from a person's ear to loop over the top of the person's head.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

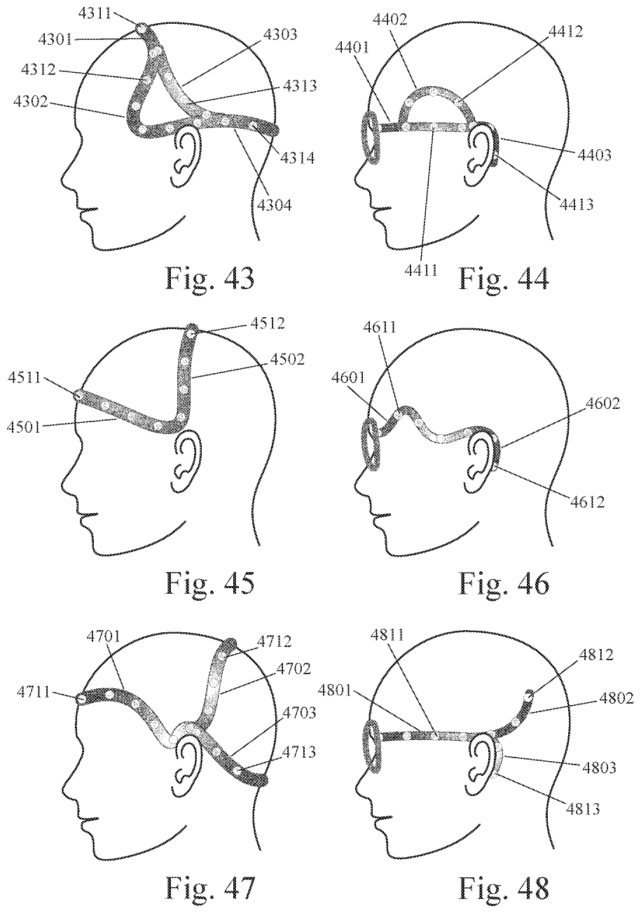

FIG. 43 shows the left side of a wearable device for measuring electromagnetic brain activity comprising: an upper arm/portion 4301 which loops over the top of a person's head and holds at least one first electromagnetic energy sensor 4311 (substantially located at an electrode position selected from the group consisting of FC3, FC1, and FCz); a forward-middle arm/portion 4302 which holds at least one second electromagnetic energy sensor 4312 (substantially located at an electrode position selected from the group consisting of F9, F7, F5, TP7, FT9, FC3, and T7); a rear-middle arm/portion 4303 which holds at least one third electromagnetic energy sensor 4313 (substantially located at an electrode position selected from the group consisting of TP7, FC3, and C5); and a rear arm/portion 4304 which loops around the rear of the person's head and holds at least one fourth electromagnetic energy sensor 4314 (substantially located at an electrode position selected from the group consisting of Iz, O1, Oz, PO7, P7, and TP7).

In an example, a wearable device for measuring electromagnetic brain activity can comprise: an upper arm/portion which loops over the top of a person's head and holds at least one first electromagnetic energy sensor (substantially located at an electrode position selected from the group consisting of FC3, FC1, and FCz); a forward-middle arm/portion; a rear-middle arm/portion; and a rear arm/portion which loops around the rear of the person's head.

In an example, a wearable device for measuring electromagnetic brain activity can comprise: an upper arm/portion which loops over the top of a person's head; a forward-middle arm/portion which holds at least one second electromagnetic energy sensor (substantially located at an electrode position selected from the group consisting of F9, F7, F5, TP7, FT9, FC3, and T7); a rear-middle arm/portion; and a rear arm/portion which loops around the rear of the person's head.

In an example, a wearable device for measuring electromagnetic brain activity can comprise: an upper arm/portion which loops over the top of a person's head; a forward-middle arm/portion; a rear-middle arm/portion which holds at least one third electromagnetic energy sensor (substantially located at an electrode position selected from the group consisting of TP7, FC3, and C5); and a rear arm/portion which loops around the rear of the person's head.

In an example, a wearable device for measuring electromagnetic brain activity can comprise: an upper arm/portion which loops over the top of a person's head; a forward-middle arm/portion; a rear-middle arm/portion; and a rear arm/portion which loops around the rear of the person's head and holds at least one fourth electromagnetic energy sensor (substantially located at an electrode position selected from the group consisting of Iz, O1, Oz, PO7, P7, and TP7).

In an example: the forward-middle portion can connect the upper arm/portion and the rear arm/portion at first and second locations; and the rear-middle arm/portion can also connect the upper arm/portion and the rear arm/portion at the first and second locations. In an example, the forward-middle arm/portion can be anterior to the rear-middle arm/portion. In an example, the centroid of the forward-middle arm/portion can be anterior to the centroid of the rear-middle arm/portion. In an example, the left side of this device can be shaped like arcuate capital letter “A” which has been rotated counter-clockwise between 90 and 180 degrees. In an example, the upper, rear-middle, and rear arms/portions of this device can together create a shape with an upward-facing concavity. In an example, the upper, rear-middle, and rear arms/portions of this device can together create a shape with an upward-and-rearward-facing concavity. In an example, an opening between the forward-middle arm/portion and the rear-middle arm/portion can have a sinusoidal wave shape. In an example, an opening between the forward-middle arm/portion and the rear-middle arm/portion can have the shape of a single-phase sinusoidal wave.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 44 shows the left side of a wearable device for measuring electromagnetic brain activity comprising: an eyewear sidepiece 4401 which holds at least one first electromagnetic energy sensor 4411 (substantially located at an electrode position selected from the group consisting of Nz, F9, FT9, and T7); an arc 4402 which extends upward from the eyewear sidepiece and holds at least one second electromagnetic energy sensor 4412 (substantially located at an electrode position selected from the group consisting of AF7, F5, FC5, and T7); and a rear ear arm/portion 4403 which curves around the rear of the person's ear and holds at least one third electromagnetic energy sensor 4413 (substantially located at an electrode position selected from the group consisting of P9, TP9, and TP7).

In an example, the left side of a wearable device for measuring electromagnetic brain activity can comprise: an eyewear sidepiece which holds at least one first electromagnetic energy sensor (substantially located at an electrode position selected from the group consisting of Nz, F9, FT9, and T7); an arc which extends upward from the eyewear sidepiece; and a rear ear arm/portion which curves around the rear of the person's ear.

In an example, the left side of a wearable device for measuring electromagnetic brain activity can comprise: an eyewear sidepiece; an arc which extends upward from the eyewear sidepiece and holds at least one second electromagnetic energy sensor (substantially located at an electrode position selected from the group consisting of AF7, F5, FC5, and T7); and a rear ear arm/portion which curves around the rear of the person's ear.

In an example, the left side of a wearable device for measuring electromagnetic brain activity can comprise: an eyewear sidepiece; an arc which extends upward from the eyewear sidepiece; and a rear ear arm/portion which curves around the rear of the person's ear and holds at least one third electromagnetic energy sensor (substantially located at an electrode position selected from the group consisting of P9, TP9, and TP7).

In an example, the arc can have a conic section shape. In an example, the arc can have a sinusoidal wave shape. In an example, both front and the rear of the arc can be connected to the sidepiece. In an example, the arc can extend between ½″ and 3″ above the sidepiece. In an example, the height and/or curvature of the arc can be adjusted. In an example, the height and/or curvature of the arc can be changed by moving the location of the front end or the rear end of the arc relative to the sidepiece. In an example, the height and/or curvature of the arc can be changed by sliding the connection of the front end or the rear end of the arc along the sidepiece. In an example, the arc can have a downward-facing concavity. In an example, the front end of the arc can connect to the front third of the (length of the) sidepiece and the rear end of the arc can connect to the rear third of the (length of the) sidepiece.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 45 shows the left side of a wearable device for measuring electromagnetic brain activity comprising: a front loop 4501 which loops around the front of the person's head (e.g. their forehead) and holds at least one first electromagnetic energy sensor 4511 (substantially located at an electrode position selected from the group consisting of FP1, FPz, AF7, F7, FT7, and T7); and an upper loop 4502 which loops over the top of the person's head (e.g. above their ears) and holds at least one second electromagnetic energy sensor 4512 (substantially located at an electrode position selected from the group consisting of T7, C5, C3, C1, and Cz).

In an example, a wearable device for measuring electromagnetic brain activity can comprise: a front loop which loops around the front of the person's head (e.g. their forehead) and holds at least one first electromagnetic energy sensor (substantially located at an electrode position selected from the group consisting of FP1, FPz, AF7, F7, FT7, and T7); and an upper loop which loops over the top of the person's head (e.g. above their ears).

In an example, a wearable device for measuring electromagnetic brain activity can comprise: a front loop which loops around the front of the person's head (e.g. their forehead); and an upper loop which loops over the top of the person's head (e.g. above their ears) and holds at least one second electromagnetic energy sensor (substantially located at an electrode position selected from the group consisting of T7, C5, C3, C1, and Cz).

In an example, the front loop can loop across and/or over the person's forehead. In an example, the front loop can loop across and/or over an upper front portion of the person's head. In an example, the front loop can be transparent or translucent. In an example, the front loop can tilt upward as it spans from a person's ear to the person's forehead. In an example, the front portion of the front loop can be at least 1″ higher than the rear portion of the front loop. In an example, the front loop can have a semicircular shape. In an example, the front loop can have a semielliptical shape. In an example, the front loop can have a downward-facing concavity. In an example, the front loop can have an upward-facing concavity. In an example, the upper loop can cross the top of a person's head in the vertical plane which connects a person's ears. In an example, this device can rest on the tops of a person's ears. In an example, the best-fitting plane for the front loop and the best-fitting plane for the upper loop can be substantially perpendicular to each other. In an example, the best-fitting plane for the front loop and the best-fitting plane for the upper loop can intersect at an angle between 70 and 90 degrees.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 46 shows the left side of a wearable device for measuring electromagnetic brain activity comprising: an undulating (e.g. sinusoidal) eyeglasses sidepiece 4601 which holds at least one first electromagnetic energy sensor 4611 (substantially located at an electrode position selected from the group consisting of Nz, FP1, AF7, F9, F7, FT9, and T7); and a rear ear arm/portion 4602 which curves around the rear of a person's ear and holds at least one second electromagnetic energy sensor 4612 (substantially located at an electrode position selected from the group consisting of P9, TP9, and TP7).

In an example, the left side of a wearable device for measuring electromagnetic brain activity can comprise: an undulating (e.g. sinusoidal) eyeglasses sidepiece which holds at least one first electromagnetic energy sensor (substantially located at an electrode position selected from the group consisting of Nz, FP1, AF7, F9, F7, FT9, and T7); and a rear ear arm/portion which curves around the rear of a person's ear.

In an example, the left side of a wearable device for measuring electromagnetic brain activity can comprise: an undulating (e.g. sinusoidal) eyeglasses sidepiece; and a rear ear arm/portion which curves around the rear of a person's ear and holds at least one second electromagnetic energy sensor (substantially located at an electrode position selected from the group consisting of P9, TP9, and TP7).

In an example, an undulating eyeglasses sidepiece can have a sinusoidal shape. In an example, a sidepiece can have at least two undulations. In an example, a sidepiece can comprise a complete sinusoidal cycle shape (including both phases). In an example, the front half of an eyeglasses sidepiece can have a downward-facing concavity and the rear half of the sidepiece can have an upward-facing concavity. In an example, the front half of an eyeglasses sidepiece can have an upward (sinusoidal or conic section) curve which spans a portion of the person's forehead and/or temple. In an example, the front half of an eyeglasses sidepiece can curve inward (closer to the person's head) as well as upward. In an example, the front half of an eyeglasses sidepiece can include an arcuate portion (e.g. a wave or arc) which curves inward (closer to the person's head) as well as upward.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 47 shows the left side of a wearable device for measuring electromagnetic brain activity comprising: a front arm/portion 4701 which extends forward onto the front of a person's head and holds at least one first electromagnetic energy sensor 4711 (substantially located at an electrode position selected from the group consisting of FPz, AF3, F5, FT7, T9, and T7); a upper arm/portion 4702 which extends upward onto the top of a person's head and holds at least one second electromagnetic energy sensor 4712 (substantially located at an electrode position selected from the group consisting of TP9, TP7, CP5, CP3, CP1, and CPz); a rear arm/portion 4703 which extends backward onto the rear of the person's head and holds at least one third electromagnetic energy sensor 4713 (substantially located at an electrode position selected from the group consisting of P9, TP9, and TP7).

In an example, the left side of a wearable device for measuring electromagnetic brain activity can comprise: a front arm/portion which extends forward onto the front of a person's head and holds at least one first electromagnetic energy sensor (substantially located at an electrode position selected from the group consisting of FPz, AF3, F5, FT7, T9, and T7); a upper arm/portion which extends upward onto the top of a person's head; a rear arm/portion which extends backward onto the rear of the person's head.

In an example, the left side of a wearable device for measuring electromagnetic brain activity can comprise: a front arm/portion which extends forward onto the front of a person's head; a upper arm/portion which extends upward onto the top of a person's head and holds at least one second electromagnetic energy sensor (substantially located at an electrode position selected from the group consisting of TP9, TP7, CP5, CP3, CP1, and CPz); a rear arm/portion which extends backward onto the rear of the person's head.

In an example, the left side of a wearable device for measuring electromagnetic brain activity can comprise: a front arm/portion which extends forward onto the front of a person's head; a upper arm/portion which extends upward onto the top of a person's head; a rear arm/portion which extends backward onto the rear of the person's head and holds at least one third electromagnetic energy sensor (substantially located at an electrode position selected from the group consisting of P9, TP9, and TP7).

In an example, the front arm/portion can loop entirely around the front of the person's head (e.g. from one ear to the other). In an example, the upper arm/portion can loop entirely over the top of the person's head (e.g. from one ear to the other). In an example, the upper arm/portion can loop entirely around the rear of the person's head (e.g. from one ear to the other). In an example, the front arm/portion, the upper arm/portion, and the rear arm/portion can intersection and/or connect at a location (directly) above the person's ear. In an example, the device can rest on the tops of the person's ears. In an example, the front arm/portion and the rear arm/portion can together comprise a ring which encircles the person's head. In an example, the front arm/portion and the rear arm/portion can together comprise an undulating (e.g. sinusoidal) ring. In an example, the front arm/portion and the rear arm/portion can together comprise an undulating ring with downward-facing concavities over the person's ears. In an example, the front of this ring can be higher than the rear of this ring. In an example, the virtual plane which best fits this ring can intersect a horizontal plane when the person is standing up forming a forward-facing angle between 5 and 75 degrees.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 48 shows the left side of a wearable device for measuring electromagnetic brain activity comprising: an eyeglasses sidepiece 4801 which holds at least one first electromagnetic energy sensor 4811 (substantially located at an electrode position selected from the group consisting of Nz, F9, FT9, and T7); an upward arm 4802 which extends upward and rearward from the person's ear and holds at least one second electromagnetic energy sensor 4812 (substantially located at an electrode position selected from the group consisting of P7, P5, P3, TP7, and T7); and a rear ear curve 4803 which curves around the rear of the person's ear and holds at least one third electromagnetic energy sensor 4813 (substantially located at an electrode position selected from the group consisting of P9, TP9, and TP7).

In an example, the left side of a wearable device for measuring electromagnetic brain activity can comprise: an eyeglasses sidepiece which holds at least one first electromagnetic energy sensor (substantially located at an electrode position selected from the group consisting of Nz, F9, FT9, and T7); an upward arm which extends upward and rearward from the person's ear; and a rear ear curve which curves around the rear of the person's ear.

In an example, the left side of a wearable device for measuring electromagnetic brain activity can comprise: an eyeglasses sidepiece; an upward arm which extends upward and rearward from the person's ear and holds at least one second electromagnetic energy sensor (substantially located at an electrode position selected from the group consisting of P7, P5, P3, TP7, and T7); and a rear ear curve which curves around the rear of the person's ear.

In an example, the left side of a wearable device for measuring electromagnetic brain activity can comprise: an eyeglasses sidepiece; an upward arm which extends upward and rearward from the person's ear; and a rear ear curve which curves around the rear of the person's ear and holds at least one third electromagnetic energy sensor (substantially located at an electrode position selected from the group consisting of P9, TP9, and TP7).

In an example, the upward arm can have a length between 1″ and 4″. In an example, the upward arm can have a forward-facing concavity. In an example, the upward arm can have a forward-and-upward-facing concavity. In an example, the sidepiece and the upward arm can together have an upward-facing “hockey stick” shape. In an example, the sidepiece of an eyeglasses frame can bifurcate into an upward arm (which extends upward and rearward) and a rear ear curve (which curves around the rear of an ear). In an example, this bifurcation can occur within 1″ of the (top of the) person's ear. In an example, this bifurcation can comprise a “whale tail” shape.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

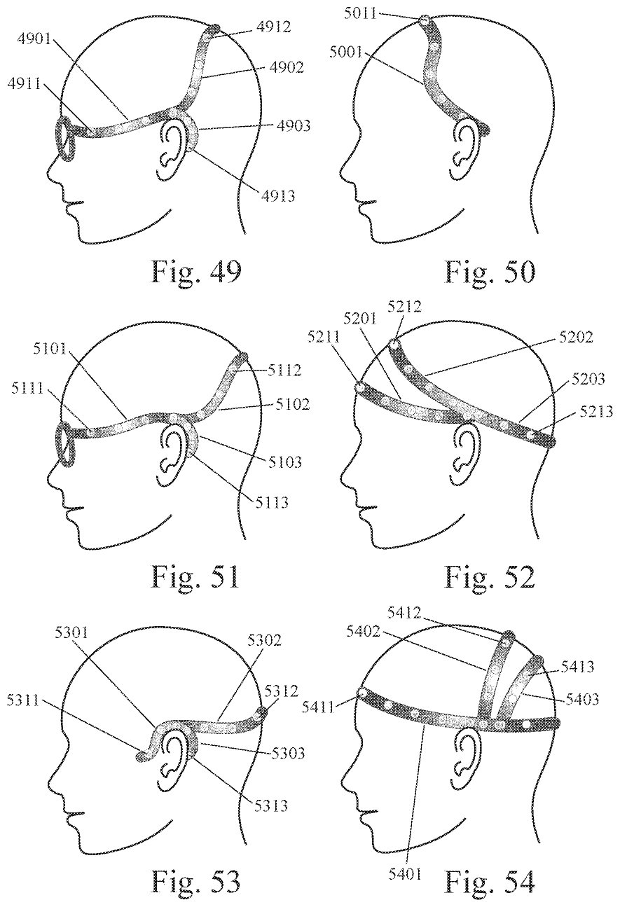

FIG. 49 shows the left side of a wearable device for measuring electromagnetic brain activity comprising: an eyewear sidepiece 4901 which holds at least one first electromagnetic energy sensor 4911 (substantially located at an electrode position selected from the group consisting of Nz, F9, FT9, and T7); an upper loop 4902 which extends upward from the rear of the sidepiece, loops around the rear half of the top of the person's head, and holds at least one second electromagnetic energy sensor 4912 (substantially located at an electrode position selected from the group consisting of TP7, CP5, CP3, CP1, CPz, and T7); and a rear ear arm/portion 4903 which curves around the rear of the person's ear and holds at least one third electromagnetic energy sensor 4913 (substantially located at an electrode position selected from the group consisting of P9, TP9, and TP7).

In an example, a wearable device for measuring electromagnetic brain activity can comprise: an eyewear sidepiece which holds at least one first electromagnetic energy sensor (substantially located at an electrode position selected from the group consisting of Nz, F9, FT9, and T7); an upper loop which extends upward from the rear of the sidepiece and loops around the rear half of the top of the person's head; and a rear ear arm/portion which curves around the rear of the person's ear.

In an example, a wearable device for measuring electromagnetic brain activity can comprise: an eyewear sidepiece; an upper loop which extends upward from the rear of the sidepiece, loops around the rear half of the top of the person's head, and holds at least one second electromagnetic energy sensor (substantially located at an electrode position selected from the group consisting of TP7, CP5, CP3, CP1, CPz, and T7); and a rear ear arm/portion which curves around the rear of the person's ear.

In an example, a wearable device for measuring electromagnetic brain activity can comprise: an eyewear sidepiece; an upper loop which extends upward from the rear of the sidepiece and loops around the rear half of the top of the person's head; and a rear ear arm/portion which curves around the rear of the person's ear and holds at least one third electromagnetic energy sensor (substantially located at an electrode position selected from the group consisting of P9, TP9, and TP7).

In an example, the eyewear sidepiece can have an upward-facing concavity. In an example, the upper loop can have a rear-facing concavity. In an example, the device can rest on the (tops of the) person's ears. In an example, the upper loop can be substantially perpendicular to the sidepiece. In an example, the upper loop can have an undulating shape.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 50 shows the left side of a wearable device for measuring electromagnetic brain activity comprising: an arcuate hairband, tiara, or headband 5001 which loops over the top of a person's head from one ear to the other ear and holds at least one first electromagnetic energy sensor 5011 (substantially located at an electrode position selected from the group consisting of TP9, FT7, FC5, FC3, FC1, FCz, and T7).

In an example, a wearable device for measuring electromagnetic brain activity can comprise: an arcuate hairband which loops over the top of a person's head from one ear to the other ear and holds at least one first electromagnetic energy sensor (substantially located at an electrode position selected from the group consisting of TP9, FT7, FC5, FC3, FC1, FCz, and T7). In an example, a wearable device for measuring electromagnetic brain activity can comprise: an arcuate tiara which loops over the top of a person's head from one ear to the other ear and holds at least one first electromagnetic energy sensor (substantially located at an electrode position selected from the group consisting of TP9, FT7, FC5, FC3, FC1, FCz, and T7). In an example, a wearable device for measuring electromagnetic brain activity can comprise: a semicircular headband which loops over the top of a person's head from one ear to the other ear and holds at least one first electromagnetic energy sensor (substantially located at an electrode position selected from the group consisting of TP9, FT7, FC5, FC3, FC1, FCz, and T7).

In an example, the device can rest on the (tops of the) person's ears. In an example, the device can further comprise projections, combs, and/or teeth which protrude between strands of the person's hair. In an example, the hairband, tiara, or headband can have a rear-facing convexity. In an example, the hairband, tiara, or headband can have a forward-facing convexity. In an example, the hairband, tiara, or headband can have a forward-facing convexity and a rear-facing convexity. In an example, the hairband, tiara, or headband can be undulating. In an example, the hairband, tiara, or headband can have a sinusoidal shape.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 51 shows the left side of a wearable device for measuring electromagnetic brain activity comprising: an eyewear sidepiece (sometimes called a “temple”) 5101 which holds at least one first electromagnetic energy sensor 5111 (substantially located at an electrode position selected from the group consisting of Nz, F9, FT9, FT7, and T7); a loop 5102 which loops around the an upper rear portion of the person' head and holds at least one second electromagnetic energy sensor 5112 (substantially located at an electrode position selected from the group consisting of P7, P5, P3, P1, Pz, TP7, and T7); and a rear ear arm/portion 5103 which curves around the rear of the person's ear and holds at least one third electromagnetic energy sensor 5113 (substantially located at an electrode position selected from the group consisting of P9, TP9, and TP7).

In an example, the left side of a wearable device for measuring electromagnetic brain activity can comprise: an eyewear sidepiece which holds at least one first electromagnetic energy sensor (substantially located at an electrode position selected from the group consisting of Nz, F9, FT9, FT7, and T7); a loop which loops around the an upper rear portion of the person' head; and a rear ear arm/portion which curves around the rear of the person's ear.

In an example, the left side of a wearable device for measuring electromagnetic brain activity can comprise: an eyewear sidepiece; a loop which loops around the an upper rear portion of the person' head and holds at least one second electromagnetic energy sensor (substantially located at an electrode position selected from the group consisting of P7, P5, P3, P1, Pz, TP7, and T7); and a rear ear arm/portion which curves around the rear of the person's ear.

In an example, the left side of a wearable device for measuring electromagnetic brain activity can comprise: an eyewear sidepiece; a loop which loops around the an upper rear portion of the person' head; and a rear ear arm/portion which curves around the rear of the person's ear and holds at least one third electromagnetic energy sensor (substantially located at an electrode position selected from the group consisting of P9, TP9, and TP7).

In an example, the loop can intersect the eyewear sidepiece at a forward-facing angle in the range of 100 to 150 degrees. In an example, the sidepiece can have a downward-facing concavity. In an example, the loop can have a rear-facing concavity. In an example, the loop can be elastic, stretchable, and/or expandable. In an example, the sidepiece and loop can be part of an undulating ring which encircles the person's head.

In an example, the right side of this mobile device (not shown) can be laterally symmetric with respect to the left side. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 52 shows the left side of a wearable device for measuring electromagnetic brain activity comprising: a lower front loop 5201 which loops around a person's forehead and holds at least one first electromagnetic energy sensor 5211 (substantially located at an electrode position selected from the group consisting of FP1, FPz, AF7, F7, FT7, and T7); an upper front loop 5202 which loops around the front of a person's head and holds at least one second electromagnetic energy sensor 5212 (substantially located at an electrode position selected from the group consisting of F3, F1, Fz, TP7, FC5, and C5), wherein the lower front loop is below the upper front loop; and a rear loop 5203 which loops around the rear of the person's head and holds at least one third electromagnetic energy sensor 5213 (substantially located at an electrode position selected from the group consisting of Iz, PO7, P7, and TP7).

In an example, a wearable device for measuring electromagnetic brain activity can comprise: a lower front loop which loops around a person's forehead and holds at least one first electromagnetic energy sensor (substantially located at an electrode position selected from the group consisting of FP1, FPz, AF7, F7, FT7, and T7); an upper front loop which loops around the front of a person's head, wherein the lower front loop is below the upper front loop; and a rear loop which loops around the rear of the person's head.

In an example, a wearable device for measuring electromagnetic brain activity can comprise: a lower front loop which loops around a person's forehead; an upper front loop which loops around the front of a person's head and holds at least one second electromagnetic energy sensor (substantially located at an electrode position selected from the group consisting of F3, F1, Fz, TP7, FC5, and C5), wherein the lower front loop is below the upper front loop; and a rear loop which loops around the rear of the person's head.

In an example, a wearable device for measuring electromagnetic brain activity can comprise: a lower front loop which loops around a person's forehead; an upper front loop which loops around the front of a person's head, wherein the lower front loop is below the upper front loop; and a rear loop which loops around the rear of the person's head and holds at least one third electromagnetic energy sensor (substantially located at an electrode position selected from the group consisting of Iz, PO7, P7, and TP7).

In an example, the upper front loop and the rear loop can together comprise a ring which encircles the person's head. In an example, the front of this ring can be tilted upward relative to the rear of this ring. In an example the upper front loop and the rear loop can be linearly aligned where they connect to each other and/or merge into each other. In an example the upper front loop can be higher than the rear loop. In an example, this device can comprise a bifurcating ring which encircles a person's head. In an example, a ring can bifurcate into an upper front loop and a lower front loop from a location within 1″ of the person's ear. In an example, the upper front loop can comprise two segments which can be reversibly connected to each other. In an example, the lower front loop can comprise two segments which can be reversibly connected to each other. In an example, the rear loop can comprise two segments which can be reversibly connected to each other. In an example, this device can rest on the (tops of the) person's ears. In an example, the upper front loop can have an upward-facing convexity. In an example, the lower front loop can have an upward-facing convexity.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 53 shows the left side of a wearable device for measuring electromagnetic brain activity comprising: a front ear arm/portion 5301 which extends 1″ to 3″ forward from the front of a person's ear and holds at least one first electromagnetic energy sensor 5311 (substantially located at an electrode position selected from the group consisting of TP7, T9, and T7); a rear loop 5302 which loops from the person's ear around the rear of the person's head and holds at least one second electromagnetic energy sensor 5312 (substantially located at an electrode position selected from the group consisting of O1, Oz, PO7, P7, TP7, and T7); and a rear ear arm/portion 5303 which curves around the rear of the person's ear and holds at least one third electromagnetic energy sensor 5313 (substantially located at an electrode position selected from the group consisting of P9, TP9, and TP7).

In an example, a wearable device for measuring electromagnetic brain activity can comprise: a front ear arm/portion which extends 1″ to 3″ forward from the front of a person's ear and holds at least one first electromagnetic energy sensor (substantially located at an electrode position selected from the group consisting of TP7, T9, and T7); a rear loop which loops from the person's ear around the rear of the person's head; and a rear ear arm/portion which curves around the rear of the person's ear.

In an example, a wearable device for measuring electromagnetic brain activity can comprise: a front ear arm/portion which extends 1″ to 3″ forward from the front of a person's ear; a rear loop which loops from the person's ear around the rear of the person's head and holds at least one second electromagnetic energy sensor (substantially located at an electrode position selected from the group consisting of O1, Oz, PO7, P7, TP7, and T7); and a rear ear arm/portion which curves around the rear of the person's ear.

In an example, a wearable device for measuring electromagnetic brain activity can comprise: a front ear arm/portion which extends 1″ to 3″ forward from the front of a person's ear; a rear loop which loops from the person's ear around the rear of the person's head; and a rear ear arm/portion which curves around the rear of the person's ear and holds at least one third electromagnetic energy sensor (substantially located at an electrode position selected from the group consisting of P9, TP9, and TP7).

In an example, the rear loop can curve upward as it extends toward the rear of the person's head. In an example, the rear loop can have an undulating shape. In an example, the rear loop can have an upward-facing convexity. In an example, the rear loop can loop around the rear of the person's head at substantially the height of the top of the person's ear. In an example, the front ear arm/portion can extend forward along a height which is substantially the height of the middle of the person's ear. In an example, the front ear arm/portion can be arcuate. In an example, the front ear arm/portion can be sinusoidal.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 54 shows the left side of a wearable device for measuring electromagnetic brain activity comprising: a ring (such as a headband) 5401 which encircles a person's head and holds at least one first electromagnetic energy sensor 5411 (substantially located at an electrode position selected from the group consisting of FP1, FPz, AF7, F7, TP7, FT7, T7, P7, P5, P3, P1, and Pz); a first upper loop 5402 which extends from the ring over the top of the person's head and holds at least one second electromagnetic energy sensor 5412 (substantially located at an electrode position selected from the group consisting of O1, Oz, PO7, P7); and a second upper loop 5403 which extends from the ring over the top of the person's head and holds at least one third electromagnetic energy sensor 5413 (substantially located at an electrode position selected from the group consisting of TP7, CP5, CP3, CP1, and CPz).

In an example, a wearable device for measuring electromagnetic brain activity can comprise: a ring (such as a headband) which encircles a person's head and holds at least one first electromagnetic energy sensor (substantially located at an electrode position selected from the group consisting of FP1, FPz, AF7, F7, TP7, FT7, T7, P7, P5, P3, P1, and Pz); a first upper loop which extends from the ring over the top of the person's head; and a second upper loop which extends from the ring over the top of the person's head.

In an example, a wearable device for measuring electromagnetic brain activity can comprise: a ring (such as a headband) which encircles a person's head; a first upper loop which extends from the ring over the top of the person's head and holds at least one second electromagnetic energy sensor (substantially located at an electrode position selected from the group consisting of O1, Oz, PO7, P7); and a second upper loop which extends from the ring over the top of the person's head.

In an example, a wearable device for measuring electromagnetic brain activity can comprise: a ring (such as a headband) which encircles a person's head; a first upper loop which extends from the ring over the top of the person's head; and a second upper loop which extends from the ring over the top of the person's head and holds at least one third electromagnetic energy sensor (substantially located at an electrode position selected from the group consisting of TP7, CP5, CP3, CP1, and CPz).

In an example, the first upper loop can be anterior to the second upper loop. In an example, the first upper loop and the second upper loop can both loop over the rear half of the top of the person's head. In an example, the first upper loop can loop over the front half of the top of the person's head and the second upper loop can both loop over the rear half of the top of the person's head. In an example, the first upper loop can have a rear-facing concavity. In an example, the first upper loop can have a forward-facing concavity and the second upper loop can have a rear-facing concavity. In an example, the device can rest on the (tops of the) person's ears.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

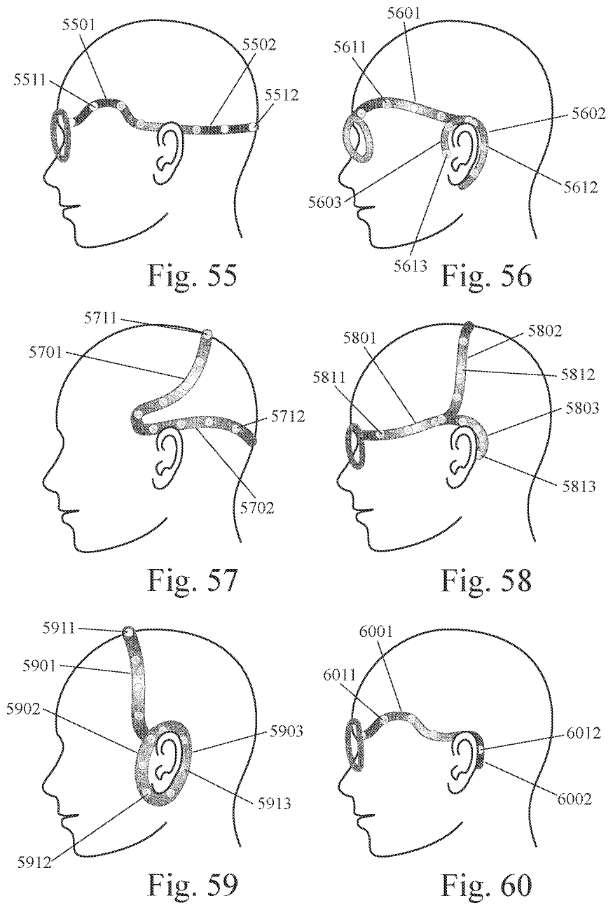

FIG. 55 shows the left side of a wearable device for measuring electromagnetic brain activity comprising: an eyewear sidepiece (e.g. eyewear “temple”) 5501 which spans from a person's ear to the front of the person's face and holds at least one first electromagnetic energy sensor 5511 (substantially located at an electrode position selected from the group consisting of Nz, AF7, F7, FT9, FT7, and T7); and a rear loop 5502 which loops from the person's ear around the rear of the person's head and holds at least one second electromagnetic energy sensor 5512 (substantially located at an electrode position selected from the group consisting of Iz, O1, Oz, PO7, P9, P7, TP9, and TP7).

In an example, a wearable device for measuring electromagnetic brain activity can comprise: an eyewear sidepiece which spans from a person's ear to the front of the person's face and holds at least one first electromagnetic energy sensor (substantially located at an electrode position selected from the group consisting of Nz, AF7, F7, FT9, FT7, and T7); and a rear loop which loops from the person's ear around the rear of the person's head.

In an example, a wearable device for measuring electromagnetic brain activity can comprise: an eyewear sidepiece which spans from a person's ear to the front of the person's face; and a rear loop which loops from the person's ear around the rear of the person's head and holds at least one second electromagnetic energy sensor (substantially located at an electrode position selected from the group consisting of Iz, O1, Oz, PO7, P9, P7, TP9, and TP7).

In an example, the sidepiece and the rear loop can be a continuous member. In an example, the rear loop can be an extension of the sidepiece rearward of the person's ear. In an example, the rear loop can loop around the rear of the person's head at substantially the height as the top of the person's ear. In an example, the sidepiece can have a downward-facing concavity. In an example, the sidepiece can include an upward curve or arc. In an example, the middle of the sidepiece can have an upward (sinusoidal or conic section shaped) wave or undulation. In an example, the device can rest on the (tops of the) person's ears. In an example, the eyewear sidepiece can span from the person's ear to an eyewear frontpiece which holds two lenses. In an example, the eyewear can be a pair of eyeglasses.

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 56 shows the left side of a wearable device for measuring electromagnetic brain activity comprising: an eyewear sidepiece 5601 which holds at least one first electromagnetic energy sensor 5611 (substantially located at an electrode position selected from the group consisting of Nz, FP1, AF7, F7, FT7 and T7); a rear ear arm/portion 5602 which curves around the rear of a person's ear and holds at least one second electromagnetic energy sensor 5612 (substantially located at an electrode position selected from the group consisting of P9, TP9, TP7); and a front ear arm/portion 5603 which curves around the front of the person's ear and holds at least one third electromagnetic energy sensor 5613 (substantially located at an electrode position selected from the group consisting of TP7, T9, and T7).

In an example, the left side of a wearable device for measuring electromagnetic brain activity can comprise: an eyewear sidepiece which holds at least one first electromagnetic energy sensor (substantially located at an electrode position selected from the group consisting of Nz, FP1, AF7, F7, FT7 and T7); a rear ear arm/portion which curves around the rear of a person's ear; and a front ear arm/portion which curves around the front of the person's ear.

In an example, the left side of a wearable device for measuring electromagnetic brain activity can comprise: an eyewear sidepiece; a rear ear arm/portion which curves around the rear of a person's ear and holds at least one second electromagnetic energy sensor substantially located at an electrode position selected from the group consisting of P9, TP9, TP7); and a front ear arm/portion which curves around the front of the person's ear.

In an example, the left side of a wearable device for measuring electromagnetic brain activity can comprise: an eyewear sidepiece; a rear ear arm/portion which curves around the rear of a person's ear; and a front ear arm/portion which curves around the front of the person's ear and holds at least one third electromagnetic energy sensor (substantially located at an electrode position selected from the group consisting of TP7, T9, and T7).

In an example, the rear ear arm/portion and the front ear arm/portion can together span between 50% and 90% of the circumference of a person's ear. In an example, the eyewear sidepiece can have a downward-facing concavity. In an example, the device can rest on the (tops of the) person's ears. In an example, the eyewear can be augmented reality eyewear.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 57 shows the left side of a wearable device for measuring electromagnetic brain activity comprising: an upper branch (or loop or arm) 5701 which loops from a position within 3″ of a person's ear around the rear half of the top of a person's head and holds at least one first electromagnetic energy sensor 5711 (substantially located at an electrode position selected from the group consisting of CP3, CP1, CPz, FT9, FT7, and C5); a lower branch (or loop or arm) 5702 which loops from the position within 3″ of the person's ear around the rear of the person's head and holds at least one second electromagnetic energy sensor 5712 (substantially located at an electrode position selected from the group consisting of Iz, O1, Oz, PO7, P7, TP7, FT9, and T7).

In an example, the left side of a wearable device for measuring electromagnetic brain activity can comprise: an upper branch which loops from a position within 3″ of a person's ear around the rear half of the top of a person's head and holds at least one first electromagnetic energy sensor (substantially located at an electrode position selected from the group consisting of CP3, CP1, CPz, FT9, FT7, and C5); a lower branch which loops from the position within 3″ of the person's ear around the rear of the person's head.

In an example, the left side of a wearable device for measuring electromagnetic brain activity can comprise: an upper branch which loops from a position within 3″ of a person's ear around the rear half of the top of a person's head; a lower branch which loops from the position within 3″ of the person's ear around the rear of the person's head and holds at least one second electromagnetic energy sensor (substantially located at an electrode position selected from the group consisting of Iz, O1, Oz, PO7, P7, TP7, FT9, and T7).

In an example, the upper branch and the lower branch can be part of the same continuous member. In an example, the upper branch can have an upward (or forward-upward) facing concavity. In an example, the lower branch can have a downward-facing concavity. In an example, the upper branch and the lower branch can diverge from each other as they span from the position near a person's ear to loop around different portions of the person's head. In an example, the upper branch and the lower branch as viewed from the left side of the person's head can have a shape like an open “bobby pin” or a cursive letter “v”. In an example, the device can rest on the (tops of the) person's ears.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 58 shows the left side of a wearable device for measuring electromagnetic brain activity comprising: an eyewear sidepiece 5801 which holds at least one first electromagnetic energy sensor 5811 (substantially located at an electrode position selected from the group consisting of Nz, F9, FT9, FT7, and T7); an upper loop 5802 which loops over the top of the person's head and holds at least one second electromagnetic energy sensor 5812 (substantially located at an electrode position selected from the group consisting of T7, C5, C3, C1, and Cz); and a rear ear arm/portion 5803 which curves around the rear of the person's outer ear and holds at least one third electromagnetic energy sensor 5813 (substantially located at an electrode position selected from the group consisting of P9, TP9, TP7, and T7).

In an example, a wearable device for measuring electromagnetic brain activity can comprise: an eyewear sidepiece which holds at least one first electromagnetic energy sensor (substantially located at an electrode position selected from the group consisting of Nz, F9, FT9, FT7, and T7); an upper loop which loops over the top of the person's head; and a rear ear arm/portion which curves around the rear of the person's outer ear.

In an example, a wearable device for measuring electromagnetic brain activity can comprise: an eyewear sidepiece; an upper loop which loops over the top of the person's head and holds at least one second electromagnetic energy sensor (substantially located at an electrode position selected from the group consisting of T7, C5, C3, C1, and Cz); and a rear ear arm/portion which curves around the rear of the person's outer ear.

In an example, a wearable device for measuring electromagnetic brain activity can comprise: an eyewear sidepiece; an upper loop which loops over the top of the person's head; and a rear ear arm/portion which curves around the rear of the person's outer ear and holds at least one third electromagnetic energy sensor (substantially located at an electrode position selected from the group consisting of P9, TP9, TP7, and T7).

In an example, the eyewear sidepiece can be arcuate. In an example, the eyewear sidepiece can be undulating (e.g. sinusoidal). In an example, the eyewear sidepiece can have a downward-facing concavity. In an example, the eyewear sidepiece can have an upward arc or wave. In an example, the middle of the eyewear sidepiece can have an upward arc or wave. In an example, the device can rest on the (tops of the) person's ears. In an example, the eyewear sidepiece can bifurcate into the upper loop and the rear ear arm/portion within 2″ of the person's ear. In an example, the upper loop can have a rear-facing concavity. In an example, the upper loop can have a forward-facing concavity. In an example, the upper loop can be (sinusoidally) undulating. In an example, the upper loop can be elastic, stretchable, and/or expandable.

In an example, the right side of this mobile device (not shown) can be laterally symmetric with respect to the left side. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 59 shows the left side of a wearable device for measuring electromagnetic brain activity comprising: an upper loop 5901 which loops over the top of a person's and holds at least one first electromagnetic energy sensor 5911 (substantially located at an electrode position selected from the group consisting of FT7, FC5, FC3, FC1, FCz, T9, and T7); a front ear arm/portion 5902 which curves around the front of a person's ear and holds at least one second electromagnetic energy sensor 5912 (substantially located at an electrode position selected from the group consisting of T9 and T7); and a rear ear arm/portion 5903 which curves around the rear of a person's ear and holds at least one third electromagnetic energy sensor 5913 (substantially located at an electrode position selected from the group consisting of P9, TP9, TP7, and T7).

In an example, a wearable device for measuring electromagnetic brain activity can comprise: an upper loop which loops over the top of a person's and holds at least one first electromagnetic energy sensor (substantially located at an electrode position selected from the group consisting of FT7, FC5, FC3, FC1, FCz, T9, and T7); a front ear arm/portion which curves around the front of a person's ear; and a rear ear arm/portion which curves around the rear of a person's ear.

In an example, a wearable device for measuring electromagnetic brain activity can comprise: an upper loop which loops over the top of a person's; a front ear arm/portion which curves around the front of a person's ear and holds at least one second electromagnetic energy sensor (substantially located at an electrode position selected from the group consisting of T9 and T7); and a rear ear arm/portion which curves around the rear of a person's ear.

In an example, a wearable device for measuring electromagnetic brain activity can comprise: an upper loop which loops over the top of a person's; a front ear arm/portion which curves around the front of a person's ear; and a rear ear arm/portion which curves around the rear of a person's ear and holds at least one third electromagnetic energy sensor (substantially located at an electrode position selected from the group consisting of P9, TP9, TP7, and T7).

In an example, the front ear arm/portion and the rear ear arm/portion can be portions of the same continuous member which encircles a person's ear. In an example, the front ear arm/portion and the rear ear arm/portion can together comprise a ring which encircles a person's ear. In an example, the front ear arm/portion and the rear ear arm/portion can together comprise a ring around a person's outer ear. In an example, this device can comprise a set of headphones. In an example, the upper loop can be undulating and/or sinusoidal.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 60 shows the left side of a wearable device for measuring electromagnetic brain activity comprising: an eyewear sidepiece 6001 which holds at least one first electromagnetic energy sensor 6011 (substantially located at an electrode position selected from the group consisting of Nz, AF7, F7, FT9, FT7, T9 and T7); and a rear ear arm/portion 6002 which curves around the back of a person's ear and holds at least one second electromagnetic energy sensor 6012 (substantially located at an electrode position selected from the group consisting of P9, TP9, TP7, and T7).

In an example, a wearable device for measuring electromagnetic brain activity can comprise: an eyewear sidepiece which holds at least one first electromagnetic energy sensor (substantially located at an electrode position selected from the group consisting of Nz, AF7, F7, FT9, FT7, T9 and T7); and a rear ear arm/portion which curves around the back of a person's ear.

In an example, a wearable device for measuring electromagnetic brain activity can comprise: an eyewear sidepiece; and a rear ear arm/portion which curves around the back of a person's ear and holds at least one second electromagnetic energy sensor (substantially located at an electrode position selected from the group consisting of P9, TP9, TP7, and T7).

In an example the rear ear arm/portion can be a continuous extension of the eyewear sidepiece. In an example, the eyewear sidepiece and the rear ear arm/portion can both be part of the same continuous member. In an example, the eyewear sidepiece can have a downward-facing concavity. In an example, the eyewear sidepiece can have an upward wave or curve. In an example, there can be an upward wave or curve in the middle of the sidepiece. In an example, the sidepiece can be undulating and/or sinusoidal. In an example, the middle of the eyewear sidepiece can curve inward (toward the person's head) as well as upward. In an example, the front of the eyewear sidepiece can curve inward (toward the person's head) as well as upward. In an example, the eyewear sidepiece can further comprise compressible foam on the body-facing side of the sidepiece. In an example, the eyewear sidepiece can further comprise a spring on the body-facing side of the sidepiece. In an example, the eyewear sidepiece can further comprise an inflatable compartment on the body-facing side of the sidepiece.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 61 shows the left side of a wearable device for measuring electromagnetic brain activity comprising: an eyeglasses frame sidepiece (e.g. “temple”) 6101 which holds at least one first electromagnetic energy sensor 6111 (substantially located at an electrode position selected from the group consisting of Nz, F9, FT9, and T7); an upward arm 6102 which extends upward from the sidepiece and holds at least one second electromagnetic energy sensor 6112 (substantially located at an electrode position selected from the group consisting of FT9, FT7, and FC5); and a rear ear arm/portion 6103 which curves around the rear of the person's ear and holds at least one third electromagnetic energy sensor 6113 (substantially located at an electrode position selected from the group consisting of P9, TP9, and TP7).

In an example, the left side of a wearable device for measuring electromagnetic brain activity can comprise: an eyeglasses frame sidepiece (e.g. “temple”) which holds at least one first electromagnetic energy sensor (substantially located at an electrode position selected from the group consisting of Nz, F9, FT9, and T7); an upward arm which extends upward from the sidepiece; and a rear ear arm/portion which curves around the rear of the person's ear.

In an example, the left side of a wearable device for measuring electromagnetic brain activity can comprise: an eyeglasses frame sidepiece (e.g. “temple”); an upward arm which extends upward from the sidepiece and holds at least one second electromagnetic energy sensor (substantially located at an electrode position selected from the group consisting of FT9, FT7, and FC5); and a rear ear arm/portion which curves around the rear of the person's ear.

In an example, the left side of a wearable device for measuring electromagnetic brain activity can comprise: an eyeglasses frame sidepiece (e.g. “temple”); and an upward arm which extends upward from the sidepiece and holds at least one second electromagnetic energy sensor (substantially located at an electrode position selected from the group consisting of FT9, FT7, and FC5).

In an example, the left side of a wearable device for measuring electromagnetic brain activity can comprise: an eyeglasses frame sidepiece (e.g. “temple”); an upward arm which extends upward from the sidepiece; and a rear ear arm/portion which curves around the rear of the person's ear and holds at least one third electromagnetic energy sensor (substantially located at an electrode position selected from the group consisting of P9, TP9, and TP7).

In an example, the upward arm can pivot or rotate up or down. In an example, the upward arm can have a first configuration in which is it substantially aligned with the sidepiece, have a second configuration in which it is substantially perpendicular to the sidepiece, and can be pivoted or rotated from its first configuration to its second. In an example, the upward arm can have a first configuration in which is it less visible, have a second configuration in which it is more visible, and can be pivoted or rotated from its first configuration to its second. In an example, the upward arm can have a forward-facing concavity. In an example, the upward arm can have a rear-facing concavity. In an example, the upward arm can extend upward from the middle (third) portion of the eyeglasses frame sidepiece. In an example, the upward arm can be between 1″ and 3″ in length.

In an example, the right side of this mobile device (not shown) can be laterally symmetric with respect to the left side. In an example, arms and/or portions of this device can be elastic, stretchable, and/or expandable. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

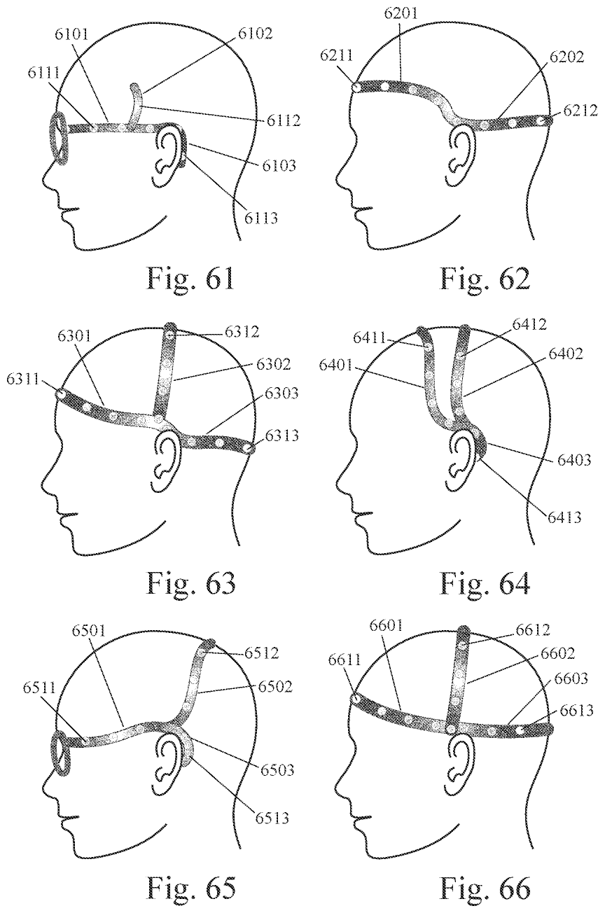

FIG. 62 shows the left side of a wearable device for measuring electromagnetic brain activity comprising: a front loop 6201 which extends forward from a person's ear, loops around the front of the person's head, and holds at least one first electromagnetic energy sensor 6211 (substantially located at an electrode position selected from the group consisting of FPz, AF3, F5, FC5, and T7); and a rear loop 6202 which extends backward from the person's ear, loops around the rear of the person's head, and holds at least one second electromagnetic energy sensor 6212 (substantially located at an electrode position selected from the group consisting of Iz, O1, Oz, PO7, P7, TP7, and T7).

In an example, a wearable device for measuring electromagnetic brain activity can comprise: a front loop which extends forward from a person's ear, loops around the front of the person's head, and holds at least one first electromagnetic energy sensor (substantially located at an electrode position selected from the group consisting of FPz, AF3, F5, FC5, and T7); and a rear loop which extends backward from the person's ear and loops around the rear of the person's head.

In an example, a wearable device for measuring electromagnetic brain activity can comprise: a front loop which extends forward from a person's ear and loops around the front of the person's head; and a rear loop which extends backward from the person's ear, loops around the rear of the person's head, and holds at least one second electromagnetic energy sensor (substantially located at an electrode position selected from the group consisting of Iz, O1, Oz, PO7, P7, TP7, and T7).

In an example, the front loop and the rear loop can be portions of the same continuous member. In an example, the front loop and the rear loop can be part of an arcuate ring which encircles the person's head. In an example, the front loop can be higher than the rear loop. In an example, the front loop can be substantially level, the rear loop can be substantially level, and the front loop can be higher than the rear loop. In an example, the device can rest on the (tops of the) person's ears. In an example, the front loop can loop across the person's forehead. In an example, the front loop can have a downward-facing concavity. In an example, the rear loop can have an upward-facing concavity. In an example, the centroid of the front loop can be higher than the centroid of the rear loop (which the person is standing up). In an example, the front loop and the rear loop can together comprise an undulating ring which encircles the person's head.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 63 shows the left side of a wearable device for measuring electromagnetic brain activity comprising: a front arm/portion 6301 which loops around the front of a person's head and holds at least one first electromagnetic energy sensor 6311 (substantially located at an electrode position selected from the group consisting of FP1, FPz, AF7, F7, FT7, and T7); an upper arm/portion 6302 which loops over the top of the person's head and holds at least one second electromagnetic energy sensor 6312 (substantially located at an electrode position selected from the group consisting of T7, C5, C3, C1, and Cz); and a rear arm/portion 6303 which loops around the rear of the person's head and holds at least one third electromagnetic energy sensor 6313 (substantially located at an electrode position selected from the group consisting of Iz, P9, TP9, and T7).

In an example, a wearable device for measuring electromagnetic brain activity can comprise: a front arm/portion which loops around the front of a person's head and holds at least one first electromagnetic energy sensor (substantially located at an electrode position selected from the group consisting of FP1, FPz, AF7, F7, FT7, and T7); an upper arm/portion which loops over the top of the person's head; and a rear arm/portion which loops around the rear of the person's head.

In an example, a wearable device for measuring electromagnetic brain activity can comprise: a front arm/portion which loops around the front of a person's head; an upper arm/portion which loops over the top of the person's head and holds at least one second electromagnetic energy sensor (substantially located at an electrode position selected from the group consisting of T7, C5, C3, C1, and Cz); and a rear arm/portion which loops around the rear of the person's head.

In an example, a wearable device for measuring electromagnetic brain activity can comprise: a front arm/portion which loops around the front of a person's head; an upper arm/portion which loops over the top of the person's head; and a rear arm/portion which loops around the rear of the person's head and holds at least one third electromagnetic energy sensor (substantially located at an electrode position selected from the group consisting of Iz, P9, TP9, and T7).

In an example, the front arm/portion and the rear arm/portion together can comprise a ring which encircles the person's head. In an example, the front arm/portion and the rear arm/portion together can comprise an undulating ring which encircles the person's head. In an example, the upper arm/portion can be substantially perpendicular to this ring. In an example, the centroid of the front arm/portion can be higher than the centroid of the rear arm/portion when the person is standing up. In an example, this ring can have a forward-upward tilt. In an example, this device can rest on the (tops of the) person's ears. In an example, the front arm/portion can have an upward-facing concavity. In an example, the front arm/portion can span the person's forehead.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 64 shows the left side of a wearable device for measuring electromagnetic brain activity comprising: a first upper loop 6401 which loops over the top of a person's head and holds at least one first electromagnetic energy sensor 6411 (substantially located at an electrode position selected from the group consisting of FT7, FC5, FC3, FC1, FCz, and T7); a second upper loops 6402 which loops over the top of the person's head and hold at least one second electromagnetic energy sensor 6412 (substantially located at an electrode position selected from the group consisting of TP7, C5, C3, C1, and Cz), wherein the first upper loop is closer to the front of the person's head than the second upper loop; and rear ear arm/portion 6403 which curves around the rear of the person's ear and holds at least one third electromagnetic energy sensor 6413 (substantially located at an electrode position selected from the group consisting of P9, TP9, and TP7).

In an example, a wearable device for measuring electromagnetic brain activity can comprise: a first upper loop which loops over the top of a person's head and holds at least one first electromagnetic energy sensor (substantially located at an electrode position selected from the group consisting of FT7, FC5, FC3, FC1, FCz, and T7); a second upper loops which loops over the top of the person's head, wherein the first upper loop is closer to the front of the person's head than the second upper loop; and rear ear arm/portion which curves around the rear of the person's ear.

In an example, a wearable device for measuring electromagnetic brain activity can comprise: a first upper loop which loops over the top of a person's head; a second upper loops which loops over the top of the person's head and holds at least one second electromagnetic energy sensor (substantially located at an electrode position selected from the group consisting of TP7, C5, C3, C1, and Cz), wherein the first upper loop is closer to the front of the person's head than the second upper loop; and rear ear arm/portion which curves around the rear of the person's ear.

In an example, a wearable device for measuring electromagnetic brain activity can comprise: a first upper loop which loops over the top of a person's head; a second upper loops which loops over the top of the person's head, wherein the first upper loop is closer to the front of the person's head than the second upper loop; and rear ear arm/portion which curves around the rear of the person's ear and holds at least one third electromagnetic energy sensor (substantially located at an electrode position selected from the group consisting of P9, TP9, and TP7).

In an example, the first upper loop and the second upper loop can connect at a location within 2″ of a person's ear. In an example, the first upper loop can have a rear-facing concavity. In an example, the first upper loop can be undulating and/or sinusoidal. In an example, the device can rest on the (tops of the) person's ears. In an example, the second upper loop can be substantially in a virtual vertical plane which connects the person's ears. In an example, the second upper loop can span the middle of the top of the person's head and the first upper loop can span the front-half of the top of the person's head.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 65 shows the left side of a wearable device for measuring electromagnetic brain activity comprising: an eyewear sidepiece 6501 which holds at least one first electromagnetic energy sensor 6511 (substantially located at an electrode position selected from the group consisting of Nz, F9, TP7, FT9, FT7, and T7); a rear-upper loop 6502 which extends rearward and upward from the rear of the sidepiece, loops over the rear half of the top of the person's head, and holds at least one second electromagnetic energy sensor 6512 (substantially located at an electrode position selected from the group consisting of TP7, CP5, CP3, CP1, CPz, and T7); and a rear ear arm/portion 6503 which curves around the rear of the person's ear and holds at least one third electromagnetic energy sensor 6513 (substantially located at an electrode position selected from the group consisting of P9, TP9, and TP7).

In an example, a wearable device for measuring electromagnetic brain activity can comprise: an eyewear sidepiece which holds at least one first electromagnetic energy sensor (substantially located at an electrode position selected from the group consisting of Nz, F9, TP7, FT9, FT7, and T7); a rear-upper loop which extends rearward and upward from the rear of the sidepiece and loops over the rear half of the top of the person's head; and a rear ear arm/portion which curves around the rear of the person's ear.

In an example, a wearable device for measuring electromagnetic brain activity can comprise: an eyewear sidepiece; a rear-upper loop which extends rearward and upward from the rear of the sidepiece, loops over the rear half of the top of the person's head, and holds at least one second electromagnetic energy sensor (substantially located at an electrode position selected from the group consisting of TP7, CP5, CP3, CP1, CPz, and T7); and a rear ear arm/portion which curves around the rear of the person's ear.

In an example, a wearable device for measuring electromagnetic brain activity can comprise: an eyewear sidepiece; a rear-upper loop which extends rearward and upward from the rear of the sidepiece and loops over the rear half of the top of the person's head; and a rear ear arm/portion which curves around the rear of the person's ear and holds at least one third electromagnetic energy sensor (substantially located at an electrode position selected from the group consisting of P9, TP9, and TP7).

In an example, the device can rest on the (tops of the) person's ears. In an example, the sidepiece can be undulating and/or sinusoidal. In an example, the sidepiece can have a downward-facing concavity. In an example, the rear-upper loop can be undulating and/or sinusoidal. In an example, read-upper loop can have a rearward-facing concavity. In an example, the sidepiece and the rear-upper loop can diverge (directly) above the person's ear.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 66 shows the left side of a wearable device for measuring electromagnetic brain activity comprising: a front arm/portion 6601 which loops around the front of a person' head and holds at least one first electromagnetic energy sensor 6611 (substantially located at an electrode position selected from the group consisting of FP1, FPz, AF7, F7, FT7, and T7); an upper arm/portion 6602 which loops over the top of the person's head and holds at least one second electromagnetic energy sensor 6612 (substantially located at an electrode position selected from the group consisting of T7, C5, C3, C1, and Cz); a rear arm/portion 6603 which loops around the rear of the person's head and holds at least one third electromagnetic energy sensor 6613 (substantially located at an electrode position selected from the group consisting of O1, Oz, PO7, P7, TP7, and T7).

In an example, a wearable device for measuring electromagnetic brain activity can comprise: a front arm/portion which loops around the front of a person' head and holds at least one first electromagnetic energy sensor (substantially located at an electrode position selected from the group consisting of FP1, FPz, AF7, F7, FT7, and T7); an upper arm/portion which loops over the top of the person's head; a rear arm/portion which loops around the rear of the person's head.

In an example, a wearable device for measuring electromagnetic brain activity can comprise: a front arm/portion which loops around the front of a person' head; an upper arm/portion which loops over the top of the person's head and holds at least one second electromagnetic energy sensor (substantially located at an electrode position selected from the group consisting of T7, C5, C3, C1, and Cz); a rear arm/portion which loops around the rear of the person's head.

In an example, a wearable device for measuring electromagnetic brain activity can comprise: a front arm/portion which loops around the front of a person' head; an upper arm/portion which loops over the top of the person's head; a rear arm/portion which loops around the rear of the person's head and holds at least one third electromagnetic energy sensor (substantially located at an electrode position selected from the group consisting of O1, Oz, PO7, P7, TP7, and T7).

In an example, the front arm/portion and the rear arm/portion can be part of the same continuous member which encircles a person's head. In an example, the front arm/portion and the rear arm/portion can together comprise a ring which encircles a person's head. In an example, the front of the ring can be higher than the rear of the ring. In an example a virtual plane which best fits the ring can intersect a horizontal plane when the person is standing up, forming a forward-facing angle between 5 and 75 degrees. In an example, the front arm/portion can further comprise two reversibly-connectable straps or bands. In an example, the rear arm/portion can further comprise two reversibly-connectable straps or bands. In an example, the upper arm/portion can further comprise two reversibly-connectable straps or bands. In an example, the front, rear, and upper arms/portions can be connected to each other (directly) above the person's ear. In an example, the device can rest on (the tops of) the person's ear.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

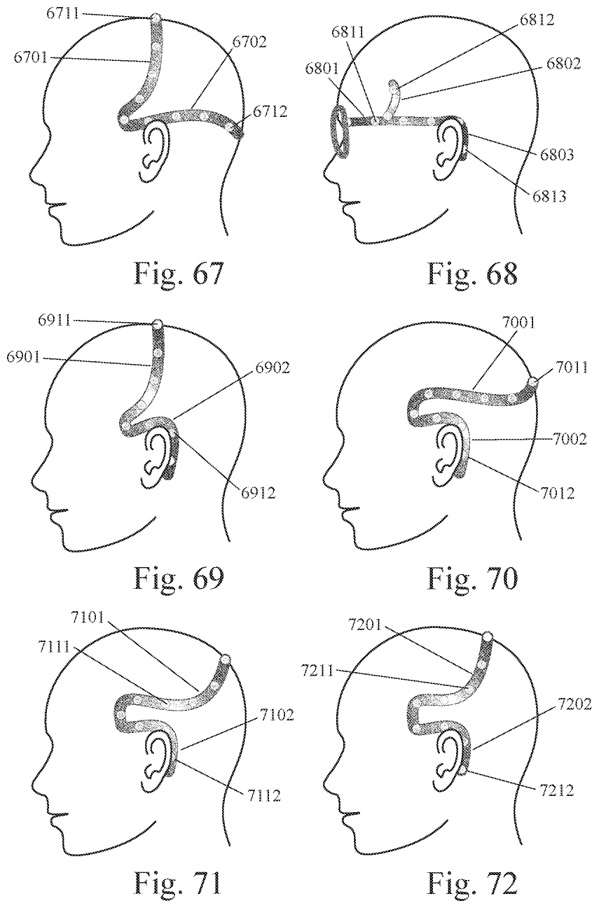

FIG. 67 shows the left side of a wearable device for measuring electromagnetic brain activity comprising: an upper arm/portion 6701 which loops over the top of a person's head and holds at least one first electromagnetic energy sensor 6711 (substantially located at an electrode position selected from the group consisting of FT9, FT7, T9, T7, C5, C3, C1, and Cz); and a rear arm/portion 6702 which loops around the rear of the person's head and holds at least one second electromagnetic energy sensor 6712 (substantially located at an electrode position selected from the group consisting of Iz, O1, Oz, PO7, P7, TP7, and T7).

In an example, a wearable device for measuring electromagnetic brain activity can comprise: an upper arm/portion which loops over the top of a person's head and holds at least one first electromagnetic energy sensor (substantially located at an electrode position selected from the group consisting of FT9, FT7, T9, T7, C5, C3, C1, and Cz); and a rear arm/portion which loops around the rear of the person's head.

In an example, a wearable device for measuring electromagnetic brain activity can comprise: an upper arm/portion which loops over the top of a person's head; and a rear arm/portion which loops around the rear of the person's head and holds at least one second electromagnetic energy sensor (substantially located at an electrode position selected from the group consisting of Iz, O1, Oz, PO7, P7, TP7, and T7).

In an example, the upper arm/portion can further comprise two reversibly-connectible straps or bands. In an example, the rear arm/portion can further comprise two reversibly-connectible straps or bands. In an example, the upper arm/portion can have a forward-facing concavity. In an example, the rear arm/portion can have a downward-facing concavity. In an example, the device can rest on the (tops of the) person's ears. In an example, the virtual plane which best fits the upper arm/portion can be substantially perpendicular to the virtual plane which best fits the rear arm/portion. In an example, the virtual plane which best fits the upper arm/portion can be substantially vertical when the person is standing up. In an example, the virtual plane which best fits the upper arm/portion can be substantially co-planar with the vertical plane which best connects the person's ears.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 68 shows the left side of eyewear for sensing brain activity comprising: a sidepiece 6801 which holds at least one first electromagnetic energy sensor 6811 (substantially located at an electrode position selected from the group consisting of Nz, F9, FT9, and T7); an upward arm 6802 which extends upward from the sidepiece and holds at least one second electromagnetic energy sensor 6812 (substantially located at an electrode position selected from the group consisting of P9, TP9, and TP7); and a rear ear arm 6803 which curves around the rear of the person's ear and holds at least one third electromagnetic energy sensor 6813.

In an example, eyewear for sensing brain activity can comprise: a sidepiece which holds at least one first electromagnetic energy sensor (substantially located at an electrode position selected from the group consisting of Nz, F9, FT9, and T7); an upward arm which extends upward from the sidepiece; and a rear ear arm which curves around the rear of the person's ear and holds at least one third electromagnetic energy sensor.

In an example, eyewear for sensing brain activity can comprise: a sidepiece; an upward arm which extends upward from the sidepiece and holds at least one second electromagnetic energy sensor (substantially located at an electrode position selected from the group consisting of P9, TP9, and TP7); and a rear ear arm which curves around the rear of the person's ear and holds at least one third electromagnetic energy sensor.

In an example, the upward arm can pivot or rotate from a first configuration in which it is lower (and in substantial alignment with the sidepiece) and a second configuration in which it is raised (and substantially perpendicular to the side piece). In an example, the upward arm can be arcuate. In an example, the upward arm can have a forward-facing concavity. In an example, the upward arm can be connected to the middle third of the sidepiece. In an example, the upward arm can be 1″ to 3″ in length. In an example, the upward arm can bend toward the person's head as is extends upward from the sidepiece. In an example, the upward can further comprise a spring, compressible foam, or an inflatable compartment.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 69 shows the left side of a wearable device for measuring electromagnetic brain activity comprising: an upper portion 6901 which extends upward from a person's ear, loops over the top of the person's head, and holds at least one first electromagnetic energy sensor 6911 (substantially located at an electrode position selected from the group consisting of FT9, FT7, T7, C5, C3, C1, and Cz); and a lower portion 6902 which curves around the rear of a person's ear and holds at least one second electromagnetic energy sensor 6912 (substantially located at an electrode position selected from the group consisting of P9, TP9, and TP7).

In an example, a wearable device for measuring electromagnetic brain activity can comprise: an upper portion which extends upward from a person's ear, loops over the top of the person's head, and holds at least one first electromagnetic energy sensor (substantially located at an electrode position selected from the group consisting of FT9, FT7, T7, C5, C3, C1, and Cz); and a lower portion which curves around the rear of a person's ear.

In an example, a wearable device for measuring electromagnetic brain activity can comprise: an upper portion which extends upward from a person's ear and loops over the top of the person's head; and a lower portion which curves around the rear of a person's ear and holds at least one second electromagnetic energy sensor (substantially located at an electrode position selected from the group consisting of P9, TP9, and TP7).

In an example, the upper portion can be arcuate. In an example, the upper portion can be undulating and/or sinusoidal. In an example, the upper portion can have a forward-facing concavity. In example, the upper portion can loop over the top of a person's head in substantially the virtual vertical plane which connects the person's ears. In an example, the device can rest on the (tops of the) person's ears. In an example, the lower portion can curve around the top and rear of the person's ear. In an example, the bottom portion can have a forward-facing concavity. In an example, the bottom portion can have a forward-and-downward-facing concavity. In an example, the device can have a forward curve or bulge where the upper and lower portions are connected to each other. In an example, the upper and lower portions can both be part of the same continuous band or strip. In an example, the virtual plane which best fits the overall device can be substantially vertical when the person is standing up.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 70 shows the left side of a wearable device for measuring electromagnetic brain activity comprising: an upper portion 7001 which loops around the rear of a person's head from a location above and forward of a person's ear and which holds at least one first electromagnetic energy sensor 7011 (substantially located at an electrode position selected from the group consisting of PO3, POz, P5, CP5, FT7, FC5, T7, and C5); and lower portion 7002 which curves around the person's ear from the location above and forward of a person's ear and which holds at least one second electromagnetic energy sensor 7012 (substantially located at an electrode position selected from the group consisting of P9, TP9, TP7, and T7).

In an example, a wearable device for measuring electromagnetic brain activity can comprise: an upper portion 7001 which loops around the rear of a person's head from a location above and forward of a person's ear and which holds at least one first electromagnetic energy sensor 7011 (substantially located at an electrode position selected from the group consisting of PO3, POz, P5, CP5, FT7, FC5, T7, and C5); and lower portion 7002 which curves around the person's ear from the location above and forward of a person's ear.

In an example, a wearable device for measuring electromagnetic brain activity can comprise: an upper portion 7001 which loops around the rear of a person's head from a location above and forward of a person's ear; and lower portion 7002 which curves around the person's ear from the location above and forward of a person's ear and which holds at least one second electromagnetic energy sensor 7012 (substantially located at an electrode position selected from the group consisting of P9, TP9, TP7, and T7).

In an example, the upper portion and the lower portion can both be part of the same continuous band or strap. In an example, the location above and forward of the person's ear can be between ½″ and 3″ above the top of the person's ear and between 1″ and 4″ in front of the top of the person's ear. In an example, the upper portion can be substantially level. In an example, the upper portion can have an upward-facing concavity. In an example, the upper portion can loop around a central portion the rear of the person's head. In an example, the lower portion can curve around the rear of the person's ear. In an example, the device can rest on the (tops of the) person's ears. In an example, the overall device, including the upper and lower portions, can have a rear-facing concavity which is located (directly) over the person's ear. In an example, the overall device can be undulating and/or sinusoidal. In an example, the middle of the left side of the upper portion can be directly above the person's left ear.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 71 shows the left side of a wearable device for measuring electromagnetic brain activity comprising: a upper portion 7101 which extends rearward and upward from a location above and forward of a person's ear, which loops over the rear half of the top of the person's head, and which holds at least one first electromagnetic energy sensor 7111 (substantially located at an electrode position selected from the group consisting of P5, P3, P1, Pz, CP5, FT7, FC5, T7, and C5); and a lower portion 7102 which extends downward and rearward from the location, which curves around the person's ear, and which holds at least one second electromagnetic energy sensor 7112 (substantially located at an electrode position selected from the group consisting of P9, TP9, TP7, and T7).

In an example, a wearable device for measuring electromagnetic brain activity can comprise: a upper portion 7101 which extends rearward and upward from a location above and forward of a person's ear, which loops over the rear half of the top of the person's head, and which holds at least one first electromagnetic energy sensor 7111 (substantially located at an electrode position selected from the group consisting of P5, P3, P1, Pz, CP5, FT7, FC5, T7, and C5); and a lower portion 7102 which extends downward and rearward from the location and which curves around the person's ear.

In an example, a wearable device for measuring electromagnetic brain activity can comprise: a upper portion 7101 which extends rearward and upward from a location above and forward of a person's ear, and which loops over the rear half of the top of the person's head; and a lower portion 7102 which extends downward and rearward from the location, which curves around the person's ear, and which holds at least one second electromagnetic energy sensor 7112 (substantially located at an electrode position selected from the group consisting of P9, TP9, TP7, and T7).

In an example, the upper portion and the lower portion can both be part of the same continuous member. In an example, the device can have a rear-facing concavity which is directly above the person's ear. In an example, the device can have a forward curve or bulge above and forward of the person's ear. In an example, the upper portion can have a forward-and-upward-facing concavity. In an example, the device can be undulating and/or sinusoidal. In an example, the device can be worn by Delenn and no one would notice. In an example, the upper and lower portions can be connected at a location over the person' temple. In an example, the upper and lower portions can be connected at a location within 1″ of the person' temple. In an example, the most-forward part of the device can be within 1″ of the person's temple.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

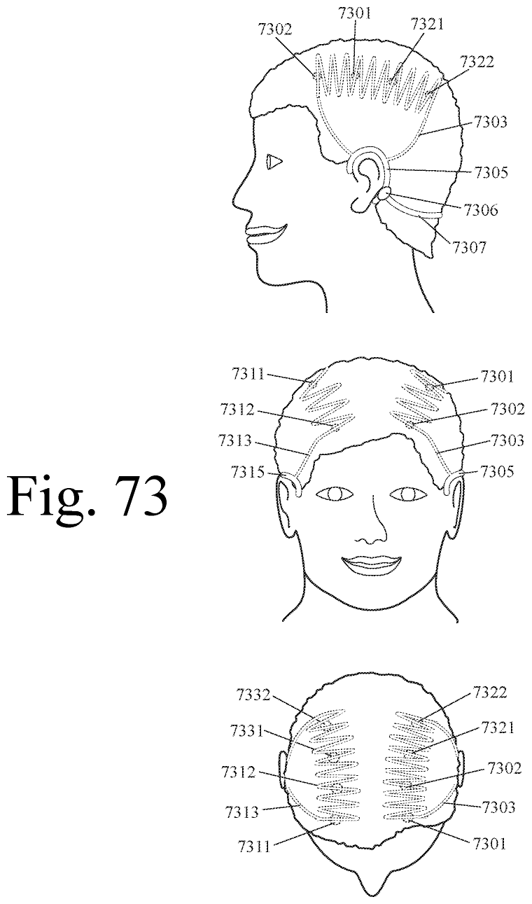

FIG. 72 shows the left side of a wearable device for measuring electromagnetic brain activity comprising: an upper arcuate portion 7201 with a rear-facing concavity which extends upward from a person's ear, loops over the top of their head, and holds at least one first electromagnetic energy sensor 7211 (substantially located at an electrode position selected from the group consisting of CP3, CP1, CPz, FT9, FT7, FC5, T7, and C5); and a lower arcuate portion 7202 which curves around the rear of the person's ear and holds at least one second electromagnetic energy sensor 7212 (substantially located at an electrode position selected from the group consisting of P9, TP9, TP7, and T7).

In an example, a wearable device for measuring electromagnetic brain activity can comprise: an upper arcuate portion with a rear-facing concavity which extends upward from a person's ear, loops over the top of their head, and holds at least one first electromagnetic energy sensor (substantially located at an electrode position selected from the group consisting of CP3, CP1, CPz, FT9, FT7, FC5, T7, and C5); and a lower arcuate portion which curves around the rear of the person's ear.

In an example, a wearable device for measuring electromagnetic brain activity can comprise: an upper arcuate portion with a rear-facing concavity which extends upward from a person's ear and loops over the top of their head; and a lower arcuate portion which curves around the rear of the person's ear and holds at least one second electromagnetic energy sensor (substantially located at an electrode position selected from the group consisting of P9, TP9, TP7, and T7).

In an example, the upper arcuate portion and the lower arcuate portion can both be part of the same continuous band or strap. In an example, the left side of the device overall (including both upper and lower portions) can have a rear-facing concavity which is located directly above the person's left ear. In an example, the device can rest on the (tops of the) person's ears. In an example, the most forward part of the device can be located over the person's temple. In an example, the most forward part of the device can be located within 1″ of the person's temple. In an example, the device can curve upwards around the rear of the person's ear, curve forward and upward to the person's temple, and then curve upward and rearward to span the rear half of the top of the person's head.

In an example, the right side of this mobile device (not shown) can be laterally symmetric with respect to the left side. In an example, arms and/or portions of this device can be elastic, stretchable, and/or expandable. In an example, arms and/or portions of this device can be pivoted or rotated around their points of intersection. In an example, one or more electromagnetic energy sensors can be attached to the arms and/or portions of this device. In an example, electroconductive areas of arms and/or portions of this device can comprise electromagnetic energy sensors. In an example, this device can further comprise a data processor, a data transmitter, and a power source. In an example, portions of this device can be transparent. Other relevant variations and components discussed in other portions of this concurrent disclosure or prior disclosures incorporated herein by reference can also be applied to this example.

FIG. 73 shows another example of wearable device for measuring electromagnetic brain activity. This example has prongs and/or teeth which slide into or under a person's hair. The top third of FIG. 73 shows this device looking at the side of a person's head. The middle third of FIG. 73 shows this device looking at the front of a person's head. The bottom third of FIG. 73 shows this device looking down at the top of a person's head.

As shown in the top third of FIG. 73, this device comprises: an arcuate frame which is configured to be worn on a person's head, wherein this arcuate frame further comprises: a left ear loop 7305 which is configured to curve around the person's left ear; a posterior loop 7307 which is connected to the left ear loop 7305, where this posterior loop 7307 is configured to curve around a posterior portion of a person's head; a left upward-extending member 7303 which is configured to loop upward toward the top of the person's head from the left ear loop 7305 and then back downward to left ear loop 7305, wherein this left upward-extending member has multiple prongs and/or teeth, wherein this left upward-extending member has a first configuration in which its upper-most portion is a first distance from the top of the person's head, wherein this left upward-extending member has a second configuration in which its upper-most portion is a second distance from the top of the person's head, wherein the second distance is less than the first distance, and wherein the prongs and/or teeth are configured to slide into or under hair in the second configuration; one or more left-side electromagnetic energy sensors, 7301, 7302, 7321, and 7322, which collect data concerning electromagnetic brain activity; and a left-side housing 7306 which contains a power source, a data processing unit, and a data transmitter and/or receiver.

The middle third and bottom third of FIG. 73 show this same device from a frontal face perspective and a top-down perspective, respectively. Right-side components include: a right ear loop 7315 which is configured to curve around the person's right ear; a right upward-extending member 7313 which is configured to loop upward toward the top of the person's head from the right ear loop 7315 and then back downward to right ear loop 7315, wherein this right upward-extending member has multiple prongs and/or teeth, wherein this right upward-extending member has a first configuration in which its upper-most portion is a first distance from the top of the person's head, wherein this right upward-extending member has a second configuration in which its upper-most portion is a second distance from the top of the person's head, wherein the second distance is less than the first distance, and wherein the prongs and/or teeth are configured to slide into or under hair in the second configuration; and one or more right-side electromagnetic energy sensors, 7311, 7312, 7331, and 7332, which collect data concerning electromagnetic brain activity. In this example, the upper tips of right and left upward-extending members are not connected or attached to each other, even in the second configuration.

In an example, a wearable device for measuring electromagnetic brain activity can comprise: a wearable band which is configured to encircle a person's head at an anterior acute angle in the range of 0 to 45 degrees with respect to a horizontal plane when the person's head is upright, wherein the band has prongs or teeth which protrude into and/or under the person's hair; and a plurality of electromagnetic energy sensors on the band; wherein one or more of the electromagnetic energy sensors are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; and wherein one or more of the electromagnetic energy sensors are made from an elastic polymer.

In an example, a wearable device for measuring electromagnetic brain activity can comprise: a wearable band which is configured to encircle a person's head at an anterior acute angle in the range of 0 to 45 degrees with respect to a horizontal plane when the person's head is upright; and a plurality of electromagnetic energy sensors on the band; wherein one or more of the electromagnetic energy sensors are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; wherein one or more of the electromagnetic energy sensors are made from a silicone material which has been impregnated, doped, coated, or embedded with metal; and wherein one or more of the electromagnetic energy sensors have prongs or teeth which protrude into and/or under the person's hair.

In an example, a wearable device for measuring electromagnetic brain activity can comprise: a wearable band which is configured to encircle a person's head at an anterior acute angle in the range of 0 to 45 degrees with respect to a horizontal plane when the person's head is upright; and a plurality of electromagnetic energy sensors on the band; wherein one or more of the electromagnetic energy sensors are located at placement sites of Modified Combinatorial Nomenclature (MCN) electrode placement system selected from the group consisting of O1, Oz, FP1, FPz, PO7, AF7, P7, F7, TP7, FT7, and T7; wherein one or more of the electromagnetic energy sensors are made from a silicone material which has been impregnated, doped, coated, or embedded with carbon; and wherein one or more of the electromagnetic energy sensors have prongs or teeth which protrude into and/or under the person's hair. Other relevant variations and components discussed in other portions of this concurrent disclosure or prior disclosures incorporated herein by reference can also be applied to this example.

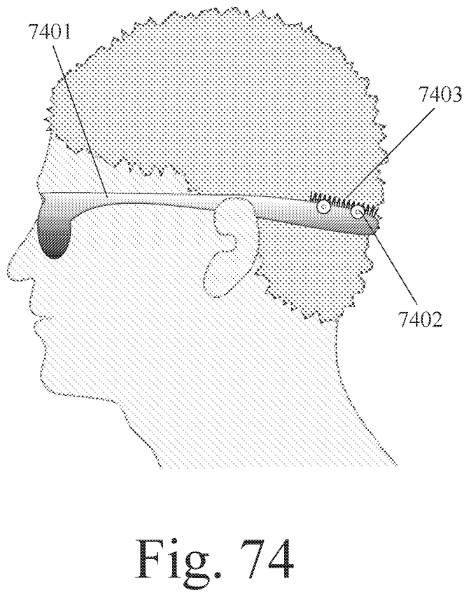

FIG. 74 shows a left-side view of another example of how this invention can be embodied in a wearable device for measuring electromagnetic brain activity. In this example, this invention is embodied as a hybrid wearable EEG monitor with an integrated eyewear frame. In this example, a wearable device for measuring electromagnetic brain activity comprises: an eyewear frame 7401 with a rear loop which loops around the rear of a person's head; a plurality of electromagnetic energy sensors (including 7402) which are held against the rear of the person's head by the eyewear frame; and prongs and/or teeth 7403 which protrude into and/or under the person's hair. In an example, the prongs and/or teeth which protrude into and/or under the person's hair can enable better electromagnetic communication between the sensors and the person's head. In an example, the electromagnetic energy sensors can be EEG sensors. In an example, this device can further comprise a power source, a data processor, and a wireless data transmitter. In an example, this device can further comprise a camera.

In this example, the anterior portion of the device serves to hold eyewear lenses and the posterior portion of the device loops completely around the back of the person's head. In this example, the front of this device rests on the bridge of the person's nose and the sides of this device rest on the person's ears. In this example, the anterior and posterior portions of this device form a continuous ring and/or band which encircles the person's head at a relatively-constant level which is just above the person's nose and ears. In this example, the eyewear portion of this device has lenses, but no display screen. In another example, the eyewear portion of this device may have a display screen, but no lenses. In another example, the eyewear portion of this device may have both a display screen and lenses. In an example, the eyewear portion of this device can serve as an augmented reality interface.

In an example, a wearable device for measuring electromagnetic brain activity can comprise: eyewear which is configured to be worn on a person's head; one or more electrodes or other brain activity sensors which are configured by the eyewear to be less than one inch from the surface of the person's head; prongs and/or teeth which protrude into and/or under the person's hair; a mobile power source and/or power transducer; a data processor; and a data transmitting member. In an example, a wearable device for measuring electromagnetic brain activity can comprise eyewear with a frame which encircles a person's head at an anterior acute angle in the range of 0 to 45 degrees with respect to a horizontal plane (when the person's head is upright) and a plurality of electromagnetic energy sensors on the eyewear frame. The electromagnetic energy sensors can be located at placement sites of Modified Combinatorial Nomenclature (MCN) electrode placement system which are selected from the group consisting of F3, F4, F7, F8, FPz, Fz, T3, T4, T5, T6, P3, P4, Pz, O1, O2, C3, C4, and Cz. In an example, the electromagnetic energy sensors can be made from a silicone material (such as polydimethylsiloxane [PDMS]) which has been impregnated, doped, coated, or embedded with conductive material (such as silver or carbon) in order to provide consistent but comfortable contact with the person's head. In an example, this device can further comprise prongs and/or teeth which protrude from the ring and/or band into and/or under the person's hair to enable. This can enable good electromagnetic communication with the person's head at locations on the person's head which are covered by hair. Other relevant variations and components discussed in other portions of this concurrent disclosure or prior disclosures incorporated herein by reference can also be applied to this example.

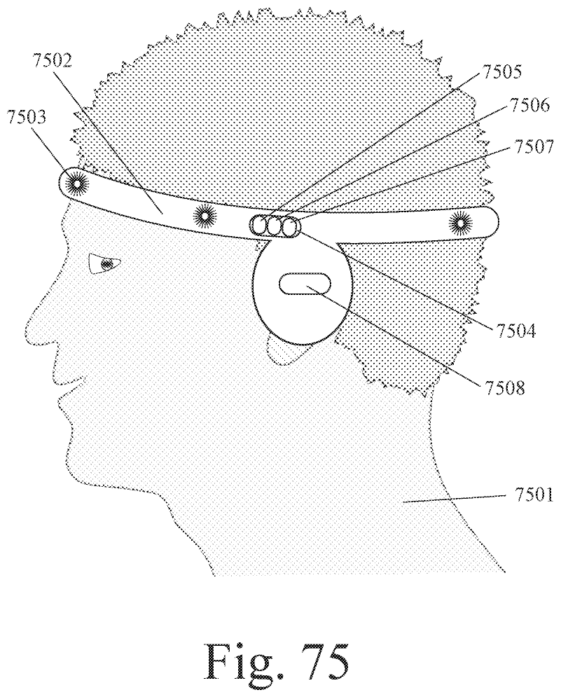

FIG. 75 shows a left-side view of an example of a wearable device for measuring electromagnetic brain activity comprising a head-worn sensor-positioning member (e.g. frame) 7502 which is configured to position a plurality of electromagnetic energy sensors including 7503 at selected locations on the person's 7501 head. In this example, sensor-positioning member (e.g. frame) 7502 is assumed to be substantially symmetric with respect to the left side (shown) and the right side (not shown) of the person's head. This monitor further comprises control unit 7504, which need not be replicated on the right side. In this example, control unit 7504 further comprises: a data processing component 7505; a power source (or transducer) 7506; and a data transmitting (and receiving) component 7507. In this example, the device further comprises a user interface 7508.

In this example, sensor-positioning member (e.g. frame) 7502 comprises: a ring or band portion which encircles the top of the person's head in a manner like the rim of a cap; and an ear-contacting portion. In an example, an ear-contacting portion can cover a person's ear like the ear-covering portion of traditional headphones. In an example, an ear-contacting portion can be at least partially inserted into a person's ear canal like an earbud or hearing aid. In this example, the ring or band portion and the ear-contacting portion are joined on the left side and right side at locations just over the person's left ear and right ear, respectively.

In an example, this device can comprise an array of electromagnetic energy sensors which are located substantially at the following set of placement sites—CP1, CP2, CP3, CP4, CP5, CP6, CPz, FC1, FC2, FC3, FC4, FC5, FC6, FCz, O1, O2, Oz, P7, P8, PO7, PO8, TP7 and TP8—or a subset of these sites. In an example, the most anterior portion of the frame of a wearable brain activity device can span a person's forehead. In an example, the most anterior portion can hold two or more electromagnetic energy sensors on a person's forehead. In an example, the most anterior portion can hold electromagnetic energy sensors at two or more locations selected from the group consisting of: FP1, FPz, and FP2. In an example, the most anterior portion can hold electromagnetic energy sensors at two or more locations selected from the group consisting of: AF7, AF3, AFz, AF4, and AF8. In an example, an ear-contacting portion can include one or more electromagnetic energy sensors which are held in electromagnetic communication with the person's outer ear, ear canal, or both.

In an example, a ring or band portion (which goes around a person's head) of this device can be circular. In an example, a ring or band portion can have a shape selected from the group consisting of: circle, ellipse, and oval. In an example, a ring or band portion can have vertical undulations or waves around a central circumferential axis, wherein the central circumferential axis is circular, elliptical, or oval. In an example, a ring or band portion can have horizontal and/or radial undulations or waves around a central circumferential axis, wherein the central circumferential axis is circular, elliptical, or oval. In an example, undulations or waves can be sinusoidal. In an example, a ring or band portion can have an ascending-and-descending wave over (and around) a person's ear. In an example, such an ascending-and-descending wave can be sinusoidal.

In an example, a ring or band portion can be generally circular or elliptical, but have an upward (concave) curved portion over (and around) the person's ear. In an example, a “ring-fitting plane” can be defined as the plane which best fits the ring or band portion of this device. In an example, the ring-fitting plane can be horizontal (when a person stands upright). In an example, the ring-fitting plane can intersect a horizontal plane at a forward-facing acute angle. In an example, this angle can be between 5 and 20 degrees. In an example, this angle can be between 15 and 45 degrees.

In an example, a sensor-positioning member (e.g. frame) can be a single continuous structure, even if it has different branches or arms. In an example, a sensor-positioning member (e.g. frame) can have multiple separately-made but connected pieces (such as branches and/or arms). In an example, these pieces can be connected with joints, hinges, or elastic straps. In an example, a sensor-positioning member (e.g. frame) can be flexible and/or elastic. In an example, the size, tension, and/or elasticity of a sensor-positioning member (e.g. frame) can be adjusted. In an example, the size, tension, and/or elasticity of a sensor-positioning member (e.g. frame) can be adjusted by a pneumatic mechanism and/or inflatable chamber. In an example, the size, tension, and/or elasticity of a sensor-positioning member (e.g. frame) can be adjusted by a hydraulic mechanism. In an example, the size, tension, and/or elasticity of a sensor-positioning member (e.g. frame) can be adjusted by a spring mechanism.

In an example, an electromagnetic energy sensor can be a capacitive electromagnetic energy sensor. In an example, an electromagnetic energy sensor can be a dry electromagnetic energy sensor. In an example, an electromagnetic energy sensor can be made with a low-conductivity material which has been doped, impregnated, or coated with a high-conductivity material. In an example, an electromagnetic energy sensor can comprise a dielectric layer of low-conductivity material between two layers of high-conductivity material. In an example, an electromagnetic energy sensor can comprise a layer of high-conductivity material between two layers of low-conductivity material. In an example, an electromagnetic energy sensor can comprise low-conductivity fibers and highly-conductive fibers which are braided or woven together.

In an example, an electromagnetic energy sensor can be made by printing high-conductivity ink onto a low-conductivity textile or fabric. In an example, an electromagnetic energy sensor can be made by printing a conductive elastomeric material onto a low-conductivity textile or fabric. In an example, an electromagnetic energy sensor can be made by melting or adhering elastomeric conductive material onto a low-conductivity textile or fabric. In an example, an electromagnetic energy sensor can be made by embroidering conductive material onto a low-conductivity textile or fabric. In an example, an electromagnetic energy sensor can be made by weaving, knitting, sewing, embroidering, layering, laminating, adhering, melting, fusing, printing, spraying, painting, or pressing electroconductive material into (or onto) a fabric or textile. In an example, electroconductive threads, fibers, yarns, strands, filaments, traces, and/or layers within a fabric or textile can be configured near a person's skin in order to receive electromagnetic energy.

In an example, an electromagnetic energy sensor can be attached to a wearable device for measuring electromagnetic brain activity using an attachment mechanism selected from the group consisting of: adhesive, band, buckle, button, channel, clasp, clip, electronic connector, flexible channel, hook, hook-and-eye mechanism, magnet, pin, plug, pocket, rivet, sewing, snap, tape, tie, and zipper. In an example, an electromagnetic energy sensor can be attached to a wearable device for measuring electromagnetic brain activity by printing, laminating, adhering, embroidering, melting, and/or sewing electroconductive material. In an example, an electronically-functional fabric or textile with electromagnetic energy sensors can be created by weaving, knitting, sewing, embroidering, layering, laminating, adhering, melting, fusing, printing, spraying, painting, or pressing together electroconductive threads, fibers, yarns, strands, filaments, traces, and/or layers. In an example, electroconductive threads, yarns, fibers, strands, channels, and/or traces comprising electromagnetic energy sensors can have shapes or configurations which are selected from the group consisting of: circular, elliptical, or other conic section; square, rectangular, hexagon, or other polygon; parallel; perpendicular; crisscrossed; nested; concentric; sinusoidal; undulating; zigzagged; and radial spokes.

In an example, an electronically-functional fabric or textile with electromagnetic energy sensors can be created by printing, spraying, or otherwise depositing electroconductive ink or resin onto an otherwise non-conductive fabric or textile. In an example, an electronically-functional circuit with electromagnetic energy sensors can be created as part of a wearable device for measuring electromagnetic brain activity by printing a conductive pattern with electroconductive ink or resin. In an example, an electronically-functional fabric or textile with electromagnetic energy sensors can be created by laminating electro-conductive members onto a non-conductive substrate. In an example, an electronically-functional fabric or textile with electromagnetic energy sensors can be created by embroidering a generally non-conductive fabric or textile member with electro-conductive members. In an example, an electronically-functional circuit with electromagnetic energy sensors can be created for a wearable device for measuring electromagnetic brain activity by embroidering a conductive pattern with electroconductive thread.

In an example, an electromagnetic energy sensor can be made with a low-conductivity material selected from the group consisting of: acetate, acrylic, cotton, denim, elastane, latex, linen, Lycra™, neoprene, nylon, nylon, polyester, wool, silicone, polydimethylsiloxane (PDMS), silk, spandex, and rayon. In an example, an electromagnetic energy sensor can be made with a high-conductivity material selected from the group consisting of: aluminum or aluminum alloy; carbon nanotubes, graphene, or other carbon-based material; copper or copper alloy; gold; nickel; silver; and steel. In an example, an electromagnetic energy sensor can be made with polydimethylsiloxane (PDMS) which has been doped or impregnated with aluminum, carbon (in one or more various configurations and formulations), copper, gold, nickel, silver, or steel. In an example, an electromagnetic energy sensor can be made from polydimethylsiloxane (PDMS) and carbon nanotubes. In an example, an electromagnetic energy sensor can be made from polydimethylsiloxane (PDMS) and silver. Other relevant variations and components discussed in other portions of this concurrent disclosure or prior disclosures incorporated herein by reference can also be applied to this example.

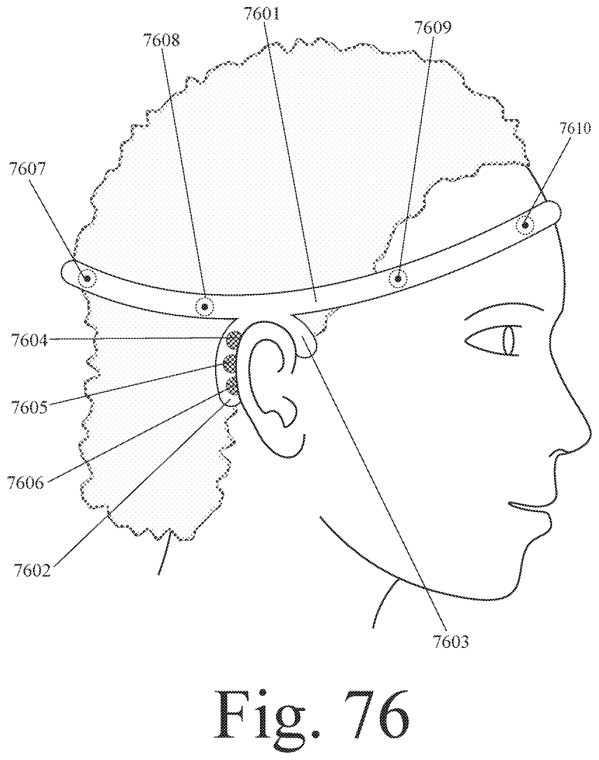

FIG. 76 shows an example of a wearable device for measuring electromagnetic brain activity comprising: a headband (or halo) 7601; a rearward ear-engaging member 7602; a frontal ear-engaging member 7603; a plurality of electromagnetic energy sensors (including 7607, 7608, 7609, and 7610) which are configured to be held in proximity to the person's head by the headband (or halo), wherein these electromagnetic energy sensors collect data concerning electromagnetic activity of the person's brain; a wireless data transmitter and/or receiver 7604; a data processor 7605; and a power source 7606. In an example, this device can have a symmetric configuration on the other side of the person's head, which is not shown here.

In an example, a headband (or halo) can encircle a person's head, including spanning at least a portion of the person's forehead. In an example, a headband (or halo) can span the center of a person's forehead. In an example, a headband (or halo) can encircle a person's head at a substantially uniform height when the person is standing upright. In an example, a headband (or halo) can encircle a person's head at a substantially uniform distance from the top of the person's head. In an example, a headband (or halo) can have a shape which is selected from the group consisting of: circle, oval, ellipse, and egg-shape.

In an example, a headband (or halo) can be made from a metal and/or a polymer. In an example, a headband (or halo) can be gas permeable (e.g. breathable) and/or liquid permeable. In an example, the perimeter of a headband (or halo) can further comprise a spring or other tensile member which holds the headband (or halo) against the surface of the person's head. In an example, the perimeter of a headband (or halo) can further comprise a spring or other tensile member which causes the headband to exert (modest) pressure against the surface of the person's head to better hold the headband on the person's head and/or better hold the electromagnetic energy sensors in proximity to the surface of the person's head.

In an example, a headband (or halo) can be stretchable, elastic, and/or expandable. In an example, one or more sections of the perimeter of a headband (or halo) can be stretchable, elastic, and/or expandable. In an example, the perimeter of a headband (or halo) can comprise one or more sections with a first degree of stretchability, elasticity, and/or expandability and one or more sections with a second degree of stretchability, elasticity, and/or expandability, wherein the second degree is greater than the first degree. In an example, the rear portion of the perimeter of a headband (spanning the rear of the person's head) can have a first degree of stretchability, elasticity, and/or expandability and the front portion of the perimeter of the headband (spanning the person's forehead) can have a second degree of stretchability, elasticity, and/or expandability, wherein the second degree is greater than the first degree. In an example, the rear portion of the perimeter of a headband (spanning the rear of the person's head) can have a first degree of stretchability, elasticity, and/or expandability and the front portion of the perimeter of the headband (spanning the person's forehead) can have a second degree of stretchability, elasticity, and/or expandability, wherein the second degree is less than the first degree.

In an example, a headband (or halo) can rest on the top portions of a person's ears (or the top portions of the tissue which connects the outer ears to the main body of the head). In an example, a rearward ear-engaging member can be configured to curve around (and frictionally engage) a rear portion of the perimeter of a person's outer ear. In an example, a rearward ear-engaging member can curve around (and frictionally engage) a person's outer ear between the 6 o'clock (210 degree) and 12 o'clock (0 degree) vectors. In an example, a rearward ear-engaging member can curve around (and frictionally engage) a person's outer ear between the 8 o'clock (210 degree) and 12 o'clock (0 degree) vectors. In an example, a rearward ear-engaging member can be configured to span between 20% and 50% of the perimeter of a person's ear. In an example, a rearward ear-engaging member can be configured to be attached to a person's earlobe.

In an example, a frontal ear-engaging member can be configured to curve around (and frictionally engage) a front portion of the perimeter of a person's outer ear. In an example, a frontal ear-engaging member can curve around (and frictionally engage) a person's outer ear between the 12 o'clock (0 degree) and 4 o'clock (120 degree) vectors. In an example, a frontal ear-engaging member can curve around (and frictionally engage) a person's outer ear between the 12 o'clock (0 degree) and 2 o'clock (60 degree) vectors. In an example, a rearward ear-engaging member can be configured to span between 10% and 30% of the perimeter of a person's ear.

FIG. 76 also shows an example of a wearable device for measuring electromagnetic brain activity comprising: a flexible ring and/or band 7601; an arcuate ear-contacting member (comprising portions 7602 and 7603); a plurality of electromagnetic energy sensors (including 7607, 7608, 7609, and 7610) which are configured to be held in proximity to the person's head by the flexible ring and/or band, wherein these electromagnetic energy sensors collect data concerning electromagnetic activity of the person's brain; a wireless data transmitter and/or receiver 7604; a data processor 7605; and a power source 7606. In an example, a wearable device for measuring electromagnetic brain activity can comprise: an arcuate flexible ring and/or band which is worn around a person's head, spanning both the person's forehead and the rear of the person's head; an arcuate ear-contacting portion which curves around at least a portion of the person's ear; and a plurality of electromagnetic energy sensors which are held by the device in electromagnetic communication with the person's head, wherein the electromagnetic energy sensors collect data concerning the person's electromagnetic brain activity.

As shown in FIG. 76, an ear-contacting portion can curve around a portion of a person's ear in an arcuate manner. In an example, the ear-contacting portion can curve around the upper portion of the person's ear in an arcuate manner. In an example, the ear-contacting portion can have a sinusoidal shape. In an example, the ear-contacting portion can have a conic section shape. In an example, the ear-contacting portion can curve around the rear portion of the person's ear in an arcuate manner. In an example, the ear-contacting portion can be attached to the person's earlobe. In an example, the ear-contacting portion can partially protrude into the person's ear canal.

In an example, the flexible band, the ear-contacting portion, and/or the electromagnetic sensors of this device can be made from an elastomeric polymer which has been impregnated, doped, coated, or embedded with conductive material. In an example, the flexible band, ear-contacting portion, and/or electromagnetic sensors of this device can be made from silicone material (such as PDMS) which has been impregnated, doped, coated, or embedded with silver. In an example, the flexible band, ear-contacting portion, and/or electromagnetic sensors of this device can be made from silicone material (such as PDMS) which has been impregnated, doped, coated, or embedded with carbon nanotubes. In an example, the flexible band, ear-contacting portion, and/or electromagnetic sensors of this device can be made from silicone material (PDMS) which has been impregnated, doped, coated, or embedded with graphene.

In an example, a headband (or halo) can be configured to receive the side frame of a pair of eyeglasses so that this headband (or halo) can be worn in combination with eyeglasses. In an example, a headband (or halo) can further comprise an opening which is configured to receive the side-piece of an eyeglass frame so that this headband (or halo) can be worn in combination with eyeglasses. In an example, a headband (or halo) can further comprise a clip or other attachment mechanism to which the side-piece of an eyeglass frame can be attached so that this headband (or halo) can be worn in combination with eyeglasses. In an example, headband (or halo) can further comprise an indentation, groove, or track into (or against) which the side-piece of the side-piece of an eyeglass frame can be placed so that this headband (or halo) can be worn in combination with eyeglasses. Other relevant variations and components discussed in other portions of this concurrent disclosure or prior disclosures incorporated herein by reference can also be applied to this example.

I claim:

1. A wearable device for measuring electromagnetic brain activity comprising:
a wearable ring and/or band which is configured to encircle a person's head at an anterior acute angle in the range of 0 to 45 degrees with respect to a horizontal plane when the person's head is upright, wherein the ring and/or band has prongs and/or teeth which protrude into and/or under the person's hair; and
a plurality of electromagnetic energy sensors on the band; wherein one or more of the electromagnetic energy sensors are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; and wherein one or more of the electromagnetic energy sensors are made from an elastic polymer.

2. A wearable device for measuring electromagnetic brain activity comprising:
a wearable ring and/or band which is configured to encircle a person's head at an anterior acute angle in the range of 0 to 45 degrees with respect to a horizontal plane when the person's head is upright; and
a plurality of electromagnetic energy sensors on the band; wherein one or more of the electromagnetic energy sensors are located at placement sites of Modified Combinatorial Nomenclature (MCN) electrode placement system selected from the group consisting of F3, F4, F7, F8, Fz, T3, T4, T5, T6, P3, P4, Pz, O1, O2, C3, C4, and Cz; wherein one or more of the electromagnetic energy sensors are made from a polymer which has been impregnated, doped, coated, or embedded with conductive material; and wherein one or more of the electromagnetic energy sensors have prongs and/or teeth which protrude into and/or under the person's hair.

3. A wearable device for measuring electromagnetic brain activity comprising:
a wearable ring and/or band which is configured to encircle a person's head at an anterior acute angle in the range of 0 to 45 degrees with respect to a horizontal plane when the person's head is upright; and
a plurality of electromagnetic energy sensors on the band; wherein one or more of the electromagnetic energy sensors are located at placement sites of Modified Combinatorial Nomenclature (MCN) electrode placement system selected from the group consisting of O1, Oz, FP1, FPz, PO7, AF7, P7, F7, TP7, FT7, and T7; wherein one or more of the electromagnetic energy sensors are made from a silicone material which has been impregnated, doped, coated, or embedded with silver or carbon; and wherein one or more of the electromagnetic energy sensors have prongs and/or teeth which protrude into and/or under the person's hair.
